SEQUENCE LISTING (69 sequences)

SEQ ID NO: 1 - protein, 32 aa, Artificial Sequence
  Zinc finger domain
XXXCXXXXCXXXXXXXXXXXXHXXXXXHXXXX

SEQ ID NO: 2 - protein, 32 aa, Artificial Sequence; identical to SEQ ID NO: 1
  Zinc finger domain

SEQ ID NO: 3 - protein, 196 aa, Artificial Sequence
  Zinc finger protein
VPIPGKKKQHICHIQGCGKVYGQSSDLQRHLRWHTGERPFMCTWSYCGKRFTRSSNLQRHKRTHTGEKKFACPECPKRFMRSDELSRHIKTHQNKKDGGGSGKKKQHICHIQGCGKVYGTTSNLRRHLRWHTGERPFMCTWSYCGKRFTRSSNLQRHKRTHTGEKKFACPECPKRFMRSDHLSRHIKTHQNKKGGS

SEQ ID NO: 4 - protein, 99 aa, Artificial Sequence
  Zinc finger protein
VPIPGKKKQHICHIQGCGKVYGTTSNLRRHLRWHTGERPFMCTWSYCGKRFTRSSNLQRHKRTHTGEKKFACPECPKRFMRSDHLSRHIKTHQNKKGGS

SEQ ID NO: 5 - protein, 99 aa, Artificial Sequence
  Zinc finger protein
MEKLRNGSGDPGKKKQHACPECGKSFSQSSNLQRHQRTHTGEKPYKCPECGKSFSRSSHLQQHQRTHTGEKPYKCPECGKSFSRSDHLSRHQRTHQNKK

SEQ ID NO: 6 - protein, 99 aa, Artificial Sequence
  Zinc finger protein
MEKLRNGSGDPGKKKQHACPECGKSFSQSSNLQRHQRTHTGEKPYKCPECGKSFSESSDLQRHQRTHTGEKPYKCPECGKSFSRSDHLSRHQRTHQNKK

SEQ ID NO: 7 - protein, 99 aa, Artificial Sequence
  Zinc finger protein
MEKLRNGSGDPGKKKQHACPECGKSFSQSSNLQRHQRTHTGEKPYKCPECGKSFSRSSHLQEHQRTHTGEKPYKCPECGKSFSRSDHLSRHQRTHQNKK

SEQ ID NO: 8 - protein, 99 aa, Artificial Sequence
  Zinc finger protein
MEKLRNGSGDPGKKKQHACPECGKSFSQSSNLQRHQRTHTGEKPYKCPECGKSFSQSSNLQRHQRTHTGEKPYKCPECGKSFSRSDHLSRHQRTHQNKK

SEQ ID NO: 9 - protein, 99 aa, Artificial Sequence
  Zinc finger protein
MEKLRNGSGDPGKKKQHACPECGKSFSQSSNLQRHQRTHTGEKPYKCPECGKSFSRSSNLQEHQRTHTGEKPYKCPECGKSFSRSDHLSRHQRTHQNKK

SEQ ID NO: 10 - protein, 99 aa, Artificial Sequence
  Zinc finger protein
MEKLRNGSGDPGKKKQHACPECGKSFSQSSNLQRHQRTHTGEKPYKCPECGKSFSQSSDLQRHQRTHTGEKPYKCPECGKSFSRSDHLSRHQRTHQNKK

SEQ ID NO: 11 - protein, 229 aa, Human
MRLAKPKAGISRSSSQGKAYENKRKTGRQREKWGMTIRFDSSFSRLRRSLDDKPYKCTECEKSFSQSSTLFQHQKIHTGKKSHKCADCGKSFFQSSNLIQHRRIHTGEKPYKCDECGESFKQSSNLIQHQRIHTGEKPYQCDECGRCFSQSSHLIQHQRTHTGEKPYQCSECGKCFSQSSHLRQHMKVHKEEKPRKTRGKNIRVKTHLPSWKAGTEGSLWLVSVKYRAF

SEQ ID NO: 12 - protein, 393 aa, Mouse
MSEEPLENAEKNPGSEEAFESGDQAERPWGDLTAEEWVSYPLQQVTDLLVHKEAHAGIRYHICSQCGKAFSQISDLNRHQKTHTGDRPYKCYECGKGFSRSSHLIQHQRTHTGERPYDCNECGKSFGRSSHLIQHQTIHTGEKPHKCTECAKASAASPHLIQHQRTHSGEKPYECEECGKSFSRSSHLAQHQRTHTGEKPYECHECGRGFSERSDLIKHYRVHTGERPYKCDECGKNFSQNSDLVRHRRAHTGEKPYHCNECGENFSRISHLVQHQRTHTGEKPYECTACGKSFSRSSHLITHQKIHTGEKPYECNECWRSFGERSDLIKHQRTHTGEKPYECVQCGKGFTQSSNLITHQRVHTGEKPYECTECDKSFSRSSALIKHKRVHTD

SEQ ID NO: 13 - protein, 28 aa, Artificial Sequence
  Zinc finger domain.
PYKCPECGKSFSXSXXLQXHQRTHTGEK

SEQ ID NO: 14 - DNA, 10 bp, Tomato golden mosaic virus
agtaaggtag

SEQ ID NO: 15 - protein, 28 aa, Artificial Sequence
  Zinc finger domain.
PYKCPECGKSFSQSDSLQRHQRTHTGEK

SEQ ID NO: 16 - protein, 28 aa, Artificial Sequence
  Zinc finger domain.
PYKCPECGKSFSRSDNLQQHQRTHTGEK

SEQ ID NO: 17 - protein, 28 aa, Artificial Sequence
  Zinc finger domain
PYKCPECGKSFSTSTHLQQHQRTHTGEK

SEQ ID NO: 18 - protein, 11 aa, Human immunodeficiency virus
YGRKKRRQRRR

SEQ ID NO: 19 - protein, 30 aa, Artificial Sequence
  Acid dimerization peptide.
AQLEKELQALEKENAQLEWELQALEKELAQ

SEQ ID NO: 20 - protein, 30 aa, Artificial Sequence
  Basic dimerization peptide
AQLKKKLQALKKKNAQLKWKLQALKKKLAQ

SEQ ID NO: 21 - protein, 20 aa, Artificial Sequence
  Flexible linker
GGGGSGGGGSGGGGSGGGGS

SEQ ID NO: 22 - DNA, 9 bp, Artificial Sequence
  Flexible linker
gcagaagcc

SEQ ID NO: 23 - protein, 5 aa, Artificial Sequence
  Flexible linker
GGGGS

SEQ ID NO: 24 - DNA, 26 bp, Artificial Sequence
  Al1 target polynucleotide
tatatataagtaaggtagtatatata

SEQ ID NO: 25 - DNA, 26 bp, Artificial Sequence
  Target polynucleotide for zinc finger protein Zif268
tatatatagcgtgggcgttatatata

SEQ ID NO: 26 - DNA, 26 bp, Artificial Sequence
  ZFP target sequence
tatatataagtaaggtagtatatata

SEQ ID NO: 27 - DNA, 26 bp, Artificial Sequence
  ZFP target sequence
tatatataagtaaggtaatatatata

SEQ ID NO: 28 - DNA, 26 bp, Artificial Sequence
  ZFP target sequence
tatatataagtaaggtattatatata

SEQ ID NO: 29 - DNA, 26 bp, Artificial Sequence
  ZFP target sequence
tatatataagtaaggtactatatata

SEQ ID NO: 30 - protein, 84 aa, Artificial Sequence
  Zinc finger protein
PYKCPECGKSFSDSXALQRHQRTHTGEKPYKCPECGKSFSQSSNLQKHQRTHTGEKPYKCPECGKSFSRSDHLQRHQRTHTGEK

SEQ ID NO: 31 - DNA, 10 bp, Artificial Sequence
  Degenerate DNA probe
ggggaannnn

SEQ ID NO: 32 - DNA, 26 bp, Artificial Sequence
  Zinc finger domain target sequence
tatatataggggaannngtatatata

SEQ ID NO: 33 - DNA, 26 bp, Artificial Sequence
  Zinc finger domain target sequence
tatatataggggaannnatatatata

SEQ ID NO: 34 - DNA, 26 bp, Artificial Sequence
  Zinc finger domain target sequence
tatatataggggaannnttatatata

SEQ ID NO: 35 - DNA, 26 bp, Artificial Sequence
  Zinc finger domain target sequence
tatatataggggaannnctatatata

SEQ ID NO: 36 - DNA, 60 bp, Artificial Sequence
  Partial zinc finger domain oligomer
ggggagaagccgtataaatgtccggaatgtggtaaaagttttagcnnnagcnnnnnnttg

SEQ ID NO: 37 - DNA, 60 bp, Artificial Sequence
  Partial zinc finger domain oligomer
tttgtatggtttttcaccggtatgggtacgctgatgnnnctgcaannnnnngctnnngct

SEQ ID NO: 38 - DNA, 60 bp, Artificial Sequence
  Partial zinc finger domain oligomer
ggtgaaaaaccatacaaatgtccagagtgcggcaaatctttctctnnntctnnnnnnctt

SEQ ID NO: 39 - DNA, 60 bp, Artificial Sequence
  Partial zinc finger domain oligomer
cttgtaaggcttctcgccagtgtgagtacgctgatgnnnctgaagnnnnnnagannnaga

SEQ ID NO: 40 - DNA, 56 bp, Artificial Sequence
  Partial zinc finger domain oligomer
ggcgagaagccttacaagtgccctgaatgcgggaagagctttagtnnnagtnnnnn

SEQ ID NO: 41 - DNA, 55 bp, Artificial Sequence
  Partial zinc finger domain oligomer
cttctcccccgtgtgcgtgcgttggtgnnnttgtaannnnnnactnnnactaaag

SEQ ID NO: 42 - DNA, 45 bp, Artificial Sequence
  PCR primer
gggcccggtctcgaattcggggagaagccgtataaatgtccggaa

SEQ ID NO: 43 - DNA, 48 bp, Artificial Sequence
  PCR primer
cccgggggtctcaagcttttacttctcccccgtgtgcgtgcgttggtg

SEQ ID NO: 44 - DNA, 10 bp, Beet curly top virus
ttgggtgctc

SEQ ID NO: 45 - DNA, 60 bp, Artificial Sequence
  Partial zinc finger domain oligomer
ggggagaagccgtataaatgtccggaatgtggtaaaagttttagcaccagcagcgatttg

SEQ ID NO: 46 - DNA, 60 bp, Artificial Sequence
  Partial zinc finger domain oligomer
tttgtatggtttttcaccggtatgggtacgctgatgacgctgcaaatcgctgctggtgct

SEQ ID NO: 47 - DNA, 60 bp, Artificial Sequence
  Partial zinc finger domain oligomer
ggtgaaaaaccatacaaatgtccagagtgcggcaaatctttctctacctctgatcatctt

SEQ ID NO: 48 - DNA, 60 bp, Artificial Sequence
  Partial zinc finger domain oligomer
cttgtaaggcttctcgccagtgtgagtacgctgatgacgctgaagatgatcagaggtaga

SEQ ID NO: 49 - DNA, 56 bp, Artificial Sequence
  Partial zinc finger domain oligomer
ggcgagaagccttacaagtgccctgaatgcgggaagagctttagtcgtagtgatag

SEQ ID NO: 50 - DNA, 55 bp, Artificial Sequence
  Partial zinc finger domain oligomer
cttctcccccgtgtgcgtgcgttggtgggtttgtaagctatcactacgactaaag

SEQ ID NO: 51 - DNA, 16 bp, Arabidopsis
atagtttacgtggcat

SEQ ID NO: 52 - DNA, 10 bp, Arabidopsis
atagtttacg

SEQ ID NO: 53 - DNA, 10 bp, Arabidopsis
tacgtggcat

SEQ ID NO: 54 - DNA, 45 bp, Artificial Sequence
  PCR primer
ttcagggcggtctctcggcttctcgccagtgtgagtacgctgatg

SEQ ID NO: 55 - DNA, 44 bp, Artificial Sequence
  PCR primer
cgaattcgggtctcagccgtataaatgtccggaatgtggtaaaa

SEQ ID NO: 56 - DNA, 45 bp, Artificial Sequence
  PCR primer
tgcggccgggtctctcggcttctcccccgtgtgcgtgcgttggtg

SEQ ID NO: 57 - DNA, 19 bp, Artificial Sequence
  ZFP target sequence
ttgggtgctttgggtgctc

SEQ ID NO: 58 - DNA, 10 bp, Artificial Sequence
  ZFP target sequence
ttgggtgctt

SEQ ID NO: 59 - DNA, 10 bp, Artificial Sequence
  ZFP target sequence
ttgggtgctc

SEQ ID NO: 60 - DNA, 35 bp, Artificial Sequence
  ZFP target probe
tatatatattgggtgctttgggtgctctatatata

SEQ ID NO: 61 - DNA, 10 bp, Artificial Sequence
  ZFP target sequence
agtaaggtag

SEQ ID NO: 62 - DNA, 10 bp, Artificial Sequence
  ZFP target sequence
ttgggtgctc

SEQ ID NO: 63 - DNA, 10 bp, Artificial Sequence
  ZFP target sequence
tacgtggcat

SEQ ID NO: 64 - DNA, 10 bp, Artificial Sequence
  ZFP target sequence
ggagatgata

SEQ ID NO: 65 - DNA, 19 bp, Artificial Sequence
  ZFP target sequence
ttgggtgctttgggtgctc

SEQ ID NO: 66 - DNA, 19 bp, Artificial Sequence
  ZFP target sequence
agtaaggtaggagatgata

SEQ ID NO: 67 - DNA, 19 bp, Artificial Sequence
  ZFP target sequence
tacgtggcattgggtgctc

SEQ ID NO: 68 - protein, 28 aa, Artificial Sequence
  Zinc finger domain
QHACPECGKSFSXSXXLQXHQRTHTGEK

SEQ ID NO: 69 - protein, 28 aa, Artificial Sequence
  Zinc finger domain
PYKCPECGKSFSXSXXLSXHQRTHTGEK

(19) United States
(12) Patent Application Publication
(10) Pub. No.: US 2003/0134350 A1
(43) Pub. Date: Jul. 17, 2003

(54) Zinc finger domain recognition code and uses thereof

(76) Inventor: Takashi Sera, San Diego, CA (US)

Correspondence Address: M. Lisa Wilson, Hale and Dorr, L.L.P., 300 Park Avenue, New York, NY, 10022, US

(21) Appl. No.: 09/911,261

(22) Filed: Jul. 23, 2001

Related U.S. Application Data
(60) Provisional application No. 60/220,060, filed on Jul. 21, 2000.

(51) Int. Cl. C12P 21/02; C12N 5/06; C07H 21/04; C12N 9/64; C12P 19/34

(52) U.S. Cl. 435/69.1; 435/91.2; 435/226; 435/320.1; 435/325; 536/23.2

(57) ABSTRACT

The present invention relates to DNA binding proteins comprising zinc finger domains in which two histidine and two cysteine residues coordinate a central zinc ion. More particularly, the invention relates to the identification of a context-independent recognition code to design zinc finger domains. This code permits identification of an amino acid for positions −1, 2, 3 and 6 of the α-helical region of the zinc finger domain from four-base pair nucleotide target sequences. The invention includes zinc finger proteins (ZFPs) designed using this recognition code, nucleic acids encoding these ZFPs and methods of using such ZFPs to modulate gene expression, alter genome structure, inhibit viral replication and detect alterations (e.g., nucleotide substitutions, deletions or insertions) in the binding sites for such proteins. In addition, the invention provides a rapid method of assembling a ZFP with three or more zinc finger domains using three sets of 256 oligonucleotides, where each set is designed to target the 256 different 4-base pair targets and allow production of all possible 3-finger ZFPs (i.e., >>10⁶) from a total of 768 oligonucleotides.

This application claims benefit of co-pending, provisional application U.S. Serial No. 60/220,060, filed Jul. 21, 2000.

### FIELD OF THE INVENTION

The present invention relates to DNA binding proteins comprising zinc finger domains in which two histidine and two cysteine residues coordinate a central zinc ion. More particularly, the invention relates to the identification of a context-independent recognition code to design zinc finger domains. This code permits identification of an amino acid for positions −1, 2, 3 and 6 of the α-helical region of the zinc finger domain from four-base pair nucleotide target sequences. The invention includes zinc finger proteins (ZFPs) designed using this recognition code, nucleic acids encoding these ZFPs and methods of using such ZFPs to modulate gene expression, alter genome structure, inhibit viral replication and detect alterations (e.g., nucleotide substitutions, deletions or insertions) in the binding sites for such proteins. In addition, the invention provides a rapid method of assembling a ZFP with three or more zinc finger domains using three sets of 256 oligonucleotides, where each set is designed to target the 256 different 4-base pair targets and allow production of all possible 3-finger ZFPs (i.e., >>10⁶) from a total of 768 oligonucleotides.

### BACKGROUND OF THE INVENTION

Selective gene expression is modulated by specific interaction of transcription factors with nucleotide sequences within the regulatory region of a gene. Zinc fingers are structural domains found in eukaryotic proteins which control gene transcription. The zinc finger domain of the Cys₂His₂ class of ZFPs is a polypeptide structural motif folded around a bound zinc ion, and has a sequence of the form -X₃-Cys-X₂₋₄-Cys-X₁₂-His-X₃₋₅-His-X₄-(SEQ ID NO: 1), wherein X is any amino acid. The zinc finger is an independent folding domain which uses a zinc ion to stabilize the packing of an antiparallel β-sheet against an α-helix. There is a great deal of sequence variation in the amino acids designated as X, however, the two consensus histidine and cysteine residues are invariant. Although most ZFPs have a similar three dimensional structure, they bind polynucleotides having a wide range of nucleotide sequences.

Several reports have discussed how zinc finger domains recognize their target polynucleotides and have attempted to generate a recognition code describing which amino acids in the zinc finger bind to which nucleotides of the target sequence. Most of these studies emphasize a three nucleotide target site. However, the limited sequence recognition information currently available largely relates to context-specific binding. In other words, the binding of the zinc finger domain is dependent on the sequence of the polynucleotides other than those which directly contact amino acids within the zinc finger domain. The present invention addresses these shortcomings and provides a context-independent zinc finger recognition code.

Further, the ability to design and artificially synthesize multi-fingered ZFPs to efficiently produce any one of many millions of choices has been limited in the art. For example, some known methods of constructing ZFPs include designing and constructing nucleic acids encoding ZFPs by phage display, random mutagenesis, combinatorial libraries, computer/rational design, affinity selection, PCR, cloning from cDNA or genomic libraries, synthetic construction and the like. (see, e.g., U.S. Pat. No. 5,786,538; Wu et al., Proc. Natl. Acad. Sci. USA 92:344-348 (1995); Jamieson et el., Biochemistry 33:5689-5695 (1994); Rebar & Pabo, Science 263:671-673 (1994); Choo & Klug, Proc. Natl. Acad. Sci. USA 91: 11168-11172 (1994); Desjarlais et al., Proc. Natl. Acad. Sci. USA 89:7345-5349 (1992); Desjarlais et al., Proc. Natl. Acad. Sci. USA 90:2256-2260 (1993); Desjarlais et al., Proc. Natl. Acad. Sci. USA 91:11099-11103; Pomerantz et al., Science 267:93-96 (1995); Pomerantz et al., Proc. Natl. Acad. Sci. USA 92:9752-9756 (1995); and Liu et al., Proc. Natl. Acad. Sci. USA 94:5525-5530 (1997); Griesman & Berg, Science 275:657-661 (1997).

Typically, a DNA is synthesized for each different individual ZFP desired, regardless of whether those proteins share some of the same domains or the number of domains in the ZFP. This can present difficulties in synthesizing large, multi-fingered ZFPs. Methods of recombinantly making ZFPs from DNA encoding individual zinc finger domains can be complicated by the difficulty of assembling the individual DNAs in the correct order, particularly when the domains have similar sequences.

Accordingly, there is a need in the art for a method to efficiently construct ZFPs comprising multiple zinc finger domains. The present invention addresses the shortcomings of the art and provides a modular method of assembling multi-fingered ZFPs from three sets of oligonucleotides encoding individual domains designed to allow the domains to assemble in the desired order.

### SUMMARY OF THE INVENTION

The present invention relates to methods of designing a zinc finger domains by identifying a 4 base-pair target sequence and determining the identity of the amino acids at positions −1, 2, 3 and 6 of the α-helix of a zinc finger domain according to the recognition code tables described herein. Any one or more domains in a multi-fingered ZFP can be designed with this method. After design, the ZFP is typically produced by recombinant methods but can also be prepared by protein synthesis methods.

The method is also useful for designing multi-fingered (i.e., multi-domained) ZFPs for longer target sequences which can be divided into overlapping 4 base pair segments, where the last base of each 4 base-pair target is the first base of the next 4 base-pair target.

In a particular embodiment, the present invention provides a method of designing a zinc finger domain of the formula

-X₃-Cys-X₂₋₄-Cys-X₅-Z⁻¹-X-Z²-Z³-X₂-Z⁶-His-X₃₋₅-His-X₄- (SEQ ID NO: 2),

wherein X is any amino acid and Xₙ represents the number of occurrences of X in the polypeptide chain, and thus X represents the framework of a Cys₂His₂ zinc finger domain. To perform this method, one (1) identifies a target nucleic acid sequence having four bases, (2) determines the identity of each X, e.g., by selecting a known zinc finger framework, a consensus framework or altering any of these framework as may be desired, and (3) determines the identity of amino acids at positions Z⁻¹, Z², Z³ and Z⁶, which are the positions of the amino acids preceding or in the α-helical portion of the zinc finger domain based on the recognition code table of the invention. Using that designed domain, a ZFP, or any other protein that is desired, can be prepared that contains that domain. The ZFP or other protein can be prepared synthetically or recombinantly, but preferably recombinantly.

The preferred recognition code table of the invention is as follows for the four base target sequence:

(i) if the first base is G, then Z⁶ is arginine, if the first base is A, then Z⁶ is glutamine, if the first base is T, then Z⁶ is threonine, tyrosine or leucine, if the first base is C, then Z⁶ is glutamic acid,

(ii) if the second base is G, then Z³ is histidine, if the second base is A, then Z³ is asparagine, if the second base is T, then Z³ is serine, if the second base is C, then Z³ is aspartic acid,

(iii) if the third base is G, then Z⁻¹ is arginine, if the third base is A, then Z⁻¹ is glutamine, if the third base is T, then Z⁻¹ is threonine or methionine, if the third base is C, then Z⁻¹ is glutamic acid,

(iv) if the complement of the fourth base is G, then Z² is serine, if the complement of the fourth base is A, then Z² is asparagine, if the complement of the fourth base is T, then Z² is threonine, and if the complement of the fourth base is C, then Z² is aspartic acid.

In a more preferred embodiment for the above recognition code, if the first base is T, then Z⁶ is threonine; and if the third base is T, then Z⁻¹ is threonine (Table 1).

In an alternative and less preferred embodiment, the recognition code table is provided as follows:

(i) if the first base is G, then Z⁶ is arginine or lysine, if the first base is A, then Z⁶ is glutamine or asparagine, if the first base is T, then Z⁶ is threonine, tyrosine, leucine, isoleucine or methionine, if the first base is C, then Z⁶ is glutamic acid or aspartic acid,

(ii) if the second base is G, then Z³ is histidine or lysine, if the second base is A, then Z³ is asparagine or glutamine, if the second base is T, then Z³ is serine, alanine or valine, if the second base is C, then Z³ is aspartic acid or glutamic acid,

(iii) if the third base is G, then Z⁻¹ is arginine or lysine, if the third base is A, then Z⁻¹ is glutamine or asparagine, if the third base is T, then Z⁻¹ is threonine, methionine leucine or isoleucine, if the third base is C, then Z⁻¹ is glutamic acid or aspartic acid,

(iv) if the complement of the fourth base is G, then Z² is serine or arginine, if the complement of the fourth base is A, then Z² is asparagine or glutamine, if the complement of the fourth base is T, then Z² is threonine, valine or alanine, and if the complement of the fourth base is C, then Z² is aspartic acid or glutamic acid.

In a preferred embodiment, the X positions of at least one of the zinc finger domains comprise the corresponding amino acids from an Sp1C or a Zif268 zinc finger domain.

The invention also provides a method to design a multi-domained ZFP, in which each zinc finger domain is independently represented by the formula above. In this case however, the target nucleic acid sequence has a length of 3N+1 base pairs, wherein N is the number of overlapping 4 base pair segments in that target and is obtained by dividing the target nucleic acid sequence into overlapping 4 base pair segments, wherein the fourth base of each segment, up to the N−1 segment, is the first base of the immediately following segment. The remainder of the design method follows that for a single domain. The method is useful for N values of 3 to 40, and more preferably where N is from 3 to 15, and when N is 3, 6, 7, 8 or 9. As for the single domain design, the X positions of at least one of the zinc finger domains can preferably comprise the corresponding amino acids from an Sp1C or a Zif268 zinc finger domain.

Another aspect of the invention provides isolated, artificial ZFPs for binding to a target nucleic acid sequence which comprise at least three zinc finger domains covalently joined to each other with from 0 to 10 amino acid residues, wherein the amino acids at positions −1, 2, 3 and 6 of the α-helix of the zinc finger are selected in accordance with a recognition code of the invention, namely at position −1, the amino acid is arginine, glutamine, threonine, methionine or glutamic acid; at position 2, the amino acid is serine, asparagine, threonine or aspartic acid; at position 3, the amino acid is histidine, asparagine, serine or aspartic acid; and at position 6, the amino acid is arginine, glutamine, threonine, tyrosine, leucine or glutamic acid; provided that the ZFP does not have an amino acid sequence consisting of any one of SEQ ID. NOS. 3-12.

In a particular embodiment, these ZFPs comprise at least three zinc finger domains, each independently represented by the formula

-X₃-Cys-X₂₋₄-Cys-X₅-Z⁻¹-X-Z²-Z³-X₂-Z⁶-His-X₃₋₅-His-X₄-,

and the domains covalently joined to each other with a from 0 to 10 amino acid residues, wherein X is any amino acid and Xₙ represents the number of occurrences of X in the polypeptide chain, wherein Z⁻¹, Z², Z³, and Z⁶ are determined by the recognition code of Table 1 with the proviso that such proteins are not those provided by any one of SEQ ID NOS 3-12. As above, X represents a framework of a Cys₂His₂ zinc finger domain and can be a known zinc finger framework, a consensus framework, a framework obtained by varying the sequence any of these frameworks or any artificial framework. Preferably known frameworks are used to determine the identities of each X.

The ZFPs of the invention comprise from 3 to 40 zinc finger domains, and preferably, 3 to 15 domains, 3 to 12 domains, 3 to 9 domains or 3 to 6 domains, as well as ZFPs with 3, 4, 5, 6, 7, 8 or 9 domains. In preferred embodiment the framework for determining X is that from Sp1C or Zif268. In one embodiment, the framework has the sequence of Sp1C domain 2, which sequence is -Pro-Tyr-Lys-Cys-Pro-Glu-Cys-Gly-Lys-Ser-Phe-Ser-Z-⁻¹-Ser-Z²-Z³-Leu-Gln-Z⁶-His-Gln-Arg-Thr-His-Thr-Gly-Glu-Lys-(SEQ ID NO: 13). Alternatively, the framework can have the sequence of Sp1C domain 1 or domain 3.

Additionally preferred ZFPs are those wherein, independently or in any combination, Z⁻¹ is methionine in at least one of said zinc finger domains; Z⁻¹ is glutamic acid in at least one of said zinc finger domains; Z² is threonine in at least one of said zinc finger domains; Z² is serine in at least one of said zinc finger domains; Z² is asparagine in at least one of said zinc finger domains; Z⁶ is glutamic acid in at least one of said zinc finger domains; Z⁶ is threonine in at least one of said zinc finger domains; Z⁶ is tyrosine in at least one of said zinc finger domains; Z⁶ is leucine in at least one of said zinc finger domains; and/or Z² is aspartic acid in at least one of said zinc finger domains, but Z⁻¹ is not arginine in the same domain. In a particular embodiment, a ZFP of the invention comprises three zinc finger domains directly joined to one to the other and each zinc finger domain represented by the formula -Pro-Tyr-Lys-Cys-Pro-Glu-Cys-Gly-Lys-Ser-Phe-Ser-Z⁻¹-Ser-Z²-Z³-Leu-Gln-Z⁶-His-Gln-Arg-Thr-His-Thr-Gly-Glu-Lys-, wherein Z⁻¹ is arginine, glutamine, threonine, methionine or glutamic acid; Z² is serine, asparagine, threonine or aspartic acid; Z³ is histidine, asparagine, serine or aspartic acid; and Z⁶ is arginine, glutamine, threonine, tyrosine, leucine or glutamic acid, and preferably, wherein Z⁻¹ is arginine, glutamine, threonine, or glutamic acid; Z² is serine, asparagine, threonine or aspartic acid; Z³ is histidine, asparagine, serine or aspartic acid; and Z⁶ is arginine, glutamine, threonine, or glutamic acid.

The ZFPs of the invention also include the 23 groups of proteins as indicated in Table 3. Groups 1-11 represent proteins that bind the following classes of nucleotide target sequences GGAM, GGTW, GGCN, GAGW, GATM, GACD, GTGW, GTAM, GTTR, GCTN and GCCD, respectively, wherein D is G, A or T; M is G or T; R is G or A; W is A or T; and N is any nucleotide. The proteins of Groups 12-23 are generally represented by the formulas AGNN, AANN, ATNN, ACNN, TGNN, TANN, TTNN, TCNN, CGNN, CANN, CTNN, and CCNN, where N, however, does not represent any nucleotide but rather represents the nucleotides for the proteins designated as belonging to the group as set forth in Table 3.

Other aspects of the invention provide isolated nucleic acids encoding the ZFPs of the invention, expression vectors comprising those nucleic acids, and host cells transformed (by any method) with the expression vectors. Among other uses, such host cells can be used in a method of preparing a ZFP by culturing the host cell for a time and under conditions to express the ZFP; and recovering the ZFP.

Yet another aspect of the invention is directed to fusion proteins with one or more of any ZFP of the invention fused to one or more proteins of interest. Likewise, the invention provides fusion proteins with one or more of any ZFP of the invention fused to one or more effector domains. The number of effector domains is preferable from one to six. Similarly, the number of ZFPs can be from one to six.

In a particular embodiment, a fusion protein has a first segment which is any ZFP of the invention, and a second segment comprising a transposase, integrase, recombinase, resolvase, invertase, protease, DNA methyltransferase, DNA demethylase, histone acetylase, histone deacetylase, nuclease, transcriptional repressor, transcriptional activator, a single-stranded DNA binding protein, a nuclear-localization signal, a transcription-protein recruiting protein or a cellular uptake domain. In an alternative embodiment, the second segments can comprise a protein domain which exhibits transposase activity, integrase activity, recombinase activity, resolvase activity, invertase activity, protease activity, DNA methyltransferase activity, DNA demethylase activity, histone acetylase activity, histone deacetylase activity, nuclease activity, nuclear localization activity, transcriptional protein recruiting activity, transcriptional repressor activity or transcriptional activator activity.

Still another aspect of the invention relates to fusion proteins which comprise a first segment which is a ZFP of the invention and a second segment comprising a protein domain capable of specifically binding to a first moiety of a divalent ligand capable of uptake by a cell. Those protein domains include but are not limited to S-protein, and S-tag, antigens, haptens and/or a single chain variable region (scFv) of an antibody. Another class of fusion proteins includes those comprising a first domain encoding single chain variable region of an antibody; a second domain enclosing a nuclear localization signal; and a third domain encoding transcriptional regulatory activity.

In addition, the invention provides isolated nucleic acids encoding any of the fusion proteins of the invention, expression vectors comprising those nucleic acids, and host cells transformed (by any method) with the expression vectors. Among other uses, such host cells can be used in a method of preparing the fusion protein by culturing the host cell for a time and under conditions to express the fusion protein; and recovering the fusion protein.

A still further aspect of the invention relates to a method of binding a target nucleic acid with artificial ZFP which comprises contacting a target nucleic acid with a ZFP of the invention or a ZFP designed in accordance with the invention in an amount and for a time sufficient for said ZFP to bind to said target nucleic acid. In a preferred embodiment the ZFP is introduced into a cell via a nucleic acid encoding said ZFP.

In particular embodiments, for the method of the preceding paragraph, as well as those additional methods of modulating expression, altering genome structure, inhibiting viral replication, creating gene insertions (knock-ins) or creating gene deletions (knock-outs), the target nucleic acid encodes, or target site is from or controls, a plant gene, a cytokine, an interleukin, an oncogene, an angiogenesis factor, a drug resistance gene and/or any other desired target, especially those provided in the detailed description of the invention. Plant genes of interest include, but are not limited to, genes from tomato, corn, rice and/or any other plant mentioned herein.

A yet further aspect of the invention provides a method of modulating expression of a gene which comprises contacting a regulatory control element of said gene with a ZFP of the invention or a ZFP designed in accordance with the invention in an amount and for a time sufficient for said ZFP to alter expression of said gene. Modulating gene expression includes both activation and repression of the gene of interest and, in one embodiment, can be done by introducing the ZFP into a cell via a nucleic acid encoding ZFP.

Another aspect of the invention relates to a method of modulating expression of a gene which comprises contacting a target nucleic acid in sufficient proximity to said gene with a fusion protein of a ZFP of the invention or a ZFP designed in accordance with the invention fused to a transcriptional regulatory domain, wherein said fusion protein contacts said nucleic acid in an amount and for a time sufficient for said transcriptional regulatory domain to alter expression of said gene. Modulating gene expression includes both activation and repression of the gene of interest and, in one embodiment, can be done by introducing the desired fusion protein into a cell via a nucleic acid encoding that fusion protein.

Yet another aspect of the invention provides a method of altering genomic structure which comprises contacting a target genomic site with a fusion protein of a ZFP of the invention or a ZFP designed in accordance with the invention fused to a protein domain which exhibits transposase activity, integrase activity, recombinase activity, DNA methyltransferase activity, DNA demethylase activity, histone acetylase activity, histone deacetylase activity or endonuclease activity, wherein the fusion protein contacts the target genomic site in an amount and for a time sufficient to alter genomic structure in or near said site. The fusion protein can also be introduced into the cell via a nucleic acid if desired. In particular embodiments, useful with direct introduction of the fusion protein into the cell, the fusion protein can comprise a cellular-uptake signal or a nuclear-localization signal.

Still another aspect of the inventions provides a method of inhibiting viral replication by introducing into a cell a nucleic acid encoding a ZFP of the invention or a ZFP designed in accordance with the invention, wherein said ZFP is competent to bind to a target site required for viral replication, and obtaining sufficient expression of the ZFP in the cell to inhibit viral replication. In one embodiment the fusion protein has a single-stranded DNA binding protein domain. While inhibition of viral replication is useful with plant viruses and animal virus, including human viruses, it can also be used with other viruses such as insect viruses or bacteriophage if desired.

Still another aspect of the invention provides a method of modulating expression of a gene by contacting a eukaryotic cell with a divalent ligand capable of uptake by the cell and having a first and second switch moiety of different specificity, wherein said cell contains

(i) a first nucleic acid expressing a first fusion protein of a ZFP of the invention or a ZFP designed in accordance with the invention specific for a target site in proximity to said gene fused to a protein domain capable of specifically binding said first switch moiety, and

(ii) a second nucleic acid expressing a second fusion protein comprising a first domain capable of specifically binding said second switch moiety, a second domain which is a nuclear localization signal and a third domain which is a transcriptional regulatory domain;

allowing said cell sufficient time to form a tertiary complex comprising said divalent ligand, said first fusion protein and said second fusion protein, to translocate said complex into the nucleus of said cell, to bind to said target site and to thereby allow said transcriptional regulatory domain to alter expression of said gene. Modulating gene expression includes both activation and repression of the gene of interest.

The protein domain capable of specifically binding the first switch moiety can be an S-protein, and S-tag or a single chain variable region (scFv) of an antibody or any derivative of these that so that binding of the respective partners can be modulated by a small molecule. The first switch moiety can be, as appropriately selected, an S-protein, an S-tag or an antigen for a single chain variable region (scFv) of an antibody. Similarly, as appropriately selected the domain capable of specifically binding the second switch moiety can be an S-protein, and S-tag or a single chain variable region (scFv) of an antibody and the second switch moiety can be an S-protein, an S-tag or an antigen for a single chain variable region (scFv) of an antibody.

A further aspect of the invention relates to artificial transposases comprising a catalytic domain, a peptide dimerization domain and a ZFP domain which is a ZFP of the invention or a ZFP designed in accordance with the invention. The transposase can also comprise a terminal inverted repeat binding domain.

Another aspect of the invention provides a method of target-specific introduction of an exogenous gene into the genome of an organism by (a) introducing into a cell a first nucleic acid encoding an artificial transposase of the invention, wherein the ZFP domain of that transposase binds a first target; a second nucleic acid encoding a second transposase of the invention, wherein the ZFP domain of that transposase binds a second target; and a third nucleic acid encoding the exogenous gene flanked by sequences capable of being bound by the terminal inverted repeat binding domain of the two transposases; and (b) forming a complex among the genome, the third nucleic acid, and the two transposases sufficient for recombination to occur and thereby introduce the exogenous gene into the genome of the organism recombination. The first and second targets can be the same or different.

Another aspect of the invention provides a method of target-specific excision an endogenous gene from the genome of an organism by (a) introducing into a cell a first nucleic acid encoding an artificial transposase of the invention, wherein the ZFP domain binds a first target; a second nucleic acid encoding a second transposase of the invention, wherein the ZFP domain binds a second target; and wherein the endogenous gene is flanked by sequences capable of being bound said ZFP domains of said transposases; and (b) forming a complex among the genome and the two transposases sufficient for recombination to occur and thereby excise the endogenous gene from the genome of the organism. The first and second targets can be the same or different.

Still a further aspect of the invention relates to diagnostic methods of using a ZFP of the invention or a ZFP designed in accordance with the invention. In one embodiment, a method for detecting an altered zinc finger recognition sequence which comprises (a) contacting a nucleic acid containing the zinc finger recognition sequence of interest with a ZFP of the invention or a ZFP designed in accordance with the invention specific for the recognition sequence, the ZFP conjugated to a signaling moiety and present in an amount sufficient to allow binding of the ZFP to the recognition sequence if said sequence was unaltered; and (b) detecting whether binding of the ZFP to the recognition sequence occurs to thereby ascertain that the recognition sequence is altered if the binding is diminished or abolished relative to binding of the ZFP to the unaltered sequence. Any detection or signaling moiety can be used including, but not limited to, a dye, biotin, streptavidin, a radioisotope and the like or a marker protein such as AP, β-gal, GUS, HRP, GFP, luciferase, and the like. The method can detect altered zinc finger recognition site with a substitution, insertion or deletion of one or more nucleotides in its sequence. In a preferred embodiment the method is used to detect single nucleotide polymorphisms (SNPs).

Still yet another aspect of the invention is directed to a method of diagnosing a disease associated with abnormal genomic structure by (a) isolating cells, blood or a tissue sample from a subject; (b) contacting nucleic acid from the cells, blood or tissue sample with a protein comprising a ZFP of the invention or a ZFP designed in accordance with the invention, a signaling moiety and, optionally, a cellular uptake domain, wherein the ZFP binds to a target site associated with said disease and has is detectable via a marker or any detection system; and (c) detecting the binding of the to the nucleic acid to thereby make the diagnosis. If desired, the amount of protein bound to the nucleic acids can be quantitated to aid in the diagnosis or to assess disease progression. In a simple method, the nucleic acid is in situ, i.e., it remains in the cells, blood or tissue sample. Alternatively, the nucleic acid can be extracted from the cells, blood or tissue samples and appropriately fixed before being contacted with the ZFP-containing protein.

Still another aspect of the invention relates to a method of making a nucleic acid encoding a ZFP comprising three contiguous zinc fingers domains, each separated from the other by no more than 10 amino acids, by

(a) preparing a mixture, under conditions for performing a polymerase-chain reaction (PCR), comprising (i) a first double-stranded oligonucleotide encoding a first zinc finger domain, (ii) a second double-stranded oligonucleotide encoding a second zinc finger domain, (iii) a third double-stranded oligonucleotide encoding a third zinc finger, (iv) a first PCR primer complementary to the 5′ end of the first oligonucleotide, (v) a second PCR primer complementary to the 3′ end of the third oligonucleotide, wherein the 3′ end of the first oligonucleotide is sufficiently complementary to the 5′ end of the second oligonucleotide to prime synthesis of said second oligonucleotide therefrom, wherein the 3′ end of the second oligonucleotide is sufficiently complementary to the 5′ end of the third oligonucleotide to prime synthesis of said third oligonucleotide therefrom, and wherein the 3′ end of the first oligonucleotide is not complementary to the 5′ end of the third oligonucleotide and the 3′ end of the second oligonucleotide is not complementary to the 5′ end of the first oligonucleotide;

(b) subjecting the mixture to a PCR; and

(c) recovering the nucleic acid encoding the three zinc finger domains and preparing a nucleic acid encoding said ZFP.

In a particular embodiment, the above method is for making a nucleic acid encoding a ZFP comprising three zinc fingers domains, each domain independently represented by the formula

-X₃-Cys-X₂₋₄-Cys-X₁₂-His-X₃₋₅-His-X₄-,

and said domains, independently, covalently joined with from 0 to 10 amino acid residues.

In these methods, the first and second PCR primers can independently include a restriction endonuclease recognition site, preferably for BbsI, BsaI, BsmBI, or BspMI, and more preferably for BsaI.

The method is particularly useful for making ZFPs comprising four or more contiguous zinc fingers domains, each separated from the other by no more than 10 amino acids. To make ZFPs with four or more domains, one (a) prepares a first nucleic acid according to the method used in preparing a ZFH with three domains, wherein the second PCR primer includes a first restriction endonuclease recognition site;

(b) prepares a second nucleic acid according to the method used in preparing a ZFP with three domains, wherein the first and second PCR primers used in this step are complementary to the 5′ and 3′ ends, respectively, flanking the number of zinc finger domains selected for amplification, wherein the first PCR primer of this step includes a restriction endonuclease recognition site that, when subjected to cleavage by its corresponding restriction endonuclease, produces an end having a sequence which is complementary to and can anneal to, the end produced when the second PCR primer of step (a) is subjected to cleavage by its corresponding restriction endonuclease and wherein the second PCR primer this step, optionally, includes a second restriction enzyme recognition site that, when subjected to cleavage produces an end that differs from and is not complementary to that produced from the first restriction endonuclease recognition site;

(c) optionally, preparing one or more additional nucleic acids by the method used in preparing a ZFP with three domains, wherein the first and second PCR primers of this step are complementary to the 5′ and 3′ ends, respectively, flanking the number of zinc finger domains selected for amplification, wherein the first PCR primer for each additional nucleic acid includes a restriction endonuclease recognition site that, when subjected to cleavage by its corresponding restriction endonuclease, produces an end having a sequence which is complementary to and can anneal to the end produced when the second PCR primer used for preparation of the second nucleic acid, or for the additional nucleic acid that is immediately upstream of the additional nucleic acid, is subjected to cleavage by its corresponding restriction endonuclease, and wherein the second PCR primer for each additional nucleic acid, optionally, includes a restriction endonuclease recognition site that, when subjected to cleavage produces an end that differs from and is not complementary to any previously used;

(d) cleaving the first nucleic acid, the second nucleic acid and the additional nucleic acids, if prepared, with their corresponding restriction endonucleases to produce cleaved first, second and additional, if prepared, nucleic acids; and

(e) combining and ligating the cleaved first, second and additional, if prepared, nucleic acids to produce the nucleic acid encoding a zinc finger protein (ZFP) having four or more zinc fingers domains.

In a particular embodiment, the above method is for making a nucleic acid encoding a zinc finger protein (ZFP) having four or more zinc fingers domains, each domain independently represented by the formula

-X₃-Cys-X₂₋₄-Cys-X₁₂-His-X₃₋₅-His-X₄-,

and the domains, independently, covalently joined with from 0 to 10 amino acid residues. In these methods each restriction endonuclease is, independently, BbsI, BsaI, BsmBI, or BspMI, and each endonuclease produces a unique pair of cleavable, anneable ends. Preferably the restriction endonuclease is BsaI and each use thereof produces a unique pair of cleavable, anneable ends. When step (c) is omitted, the nucleic acid encodes a zinc finger protein (ZFP) having four, five or six zinc finger domains, depending on the PCR amplification primers locations relative to the three domains. When the PCR amplification primers for the second nucleic acid are selected to amplify three zinc finger domains and one additional nucleic acid is prepared by step (c), then the nucleic acid encodes a zinc finger protein (ZFP) having seven, eight or nine zinc finger domains, depending on the location of PCR amplification primers in step (c) relative to the three domains of the additional nucleic acid of step(c).

The oligonucleotides used in these modular assembly methods can be provide optimal codon usage for a desired organism, such as a bacterium, a fungus, a yeast, an animal, an insect or a plant or any other organism described herein, whether transgenic or naturally occurring.

62. An expression vector comprising a nucleic acid prepared by the method of any one of claims 40-61.

63. A host cell comprising the expression vector of claim 62.

64. A method of preparing a zinc finger protein which comprises

(a) culturing the host cell of claim 63 for a time and under conditions to express said ZFP; and

(b) recovering said ZFP.

In addition, the invention provides expression vectors comprising the nucleic acids prepared by the above modular assembly methods and host cells transformed (by any method) with the expression vectors. Among other uses, such host cells can be used in a method of preparing the encoded ZFPs by culturing the host cell for a time and under conditions to express the desired ZFPs protein; and recovering those ZFPs.

Yet a further aspect of the invention provides a set of oligonucleotides comprising a number of separate oligonucleotides, each oligonucleotide encoding one zinc finger domain and the set of oligonucleotides including at least one oligonucleotide for more than half of the possible four base pair target sequences (using one of the nucleotides G, A, T, and C at each of the four positions, wherein the amino acids at positions −1, 2, 3 and 6 of the α-helix of the zinc finger are selected at position −1 as the amino acid arginine, glutamine, threonine, methionine or glutamic acid; at position 2 as the amino acid serine, asparagine, threonine or aspartic acid; at position 3 as the amino acid histidine, asparagine, serine or aspartic acid; and at position 6 as the amino acid arginine, glutamine, threonine, tyrosine, leucine or glutamic acid. The set has at least 150 oligonucleotides, and preferably the number ranges from about 200 to about 256, oligonucleotides and more preferably is 256 oligonucleotides.

In a particular embodiment, the invention provides a set of 256 separate or individually-packaged oligonucleotides, each oligonucleotide comprising a nucleotide sequence encoding one of the 256 zinc finger domains represented by the formula

-X₃-Cys-X₂₋₄-Cys-X₅-Z⁻¹-X-Z²-Z³-X₂-Z⁶-His-X₃₋₅-His-X₄-,

wherein X is any amino acid and Xₙ represents the number of occurrences of X in the polypeptide chain; Z⁻¹is arginine, glutamine, threonine, or glutamic acid; Z² is serine, asparagine, threonine or aspartic acid; Z³ is histidine, asparagine, serine or aspartic acid; and Z⁶ is arginine, glutamine, threonine, or glutamic acid. In a preferred embodiment, each X at a given position in the formula is the same in each of the 256 zinc finger domains and can be from a known zinc finger framework. The codon usage in the oligonucleotides can be also be optimized for any desired organism for which such information is available, such as, but not limited to human, mouse, rice, and E. coli.

In addition the invention provides a set of oligonucleotides for producing nucleic acid encoding ZFPs having three or more zinc finger domains, the set having three subsets of 256 separate or individually-packaged oligonucleotides, each oligonucleotide comprising a nucleotide sequence encoding one of the 256 zinc finger domains represented by the formula

-X₃-Cys-X₂₋₄-Cys-X₅-Z⁻¹-X-Z²-Z³-X₂-Z⁶-His-X₃₋₅-His-X₄-,

wherein X is any amino acid and Xₙ represents the number of occurrences of X in the polypeptide chain; Z⁻¹ is arginine, glutamine, threonine, or glutamic acid; Z² is serine, asparagine, threonine or aspartic acid; Z³ is histidine, asparagine, serine or aspartic acid; and Z⁶ is arginine, glutamine, threonine, or glutamic acid; and wherein the 3′ end of the first set oligonucleotides are sufficiently complementary to the 5′ end of the second set oligonucleotides to prime synthesis of said second set oligonucleotides therefrom, the 3′ end of the second set oligonucleotides are sufficiently complementary to the 5′ end of the third set oligonucleotides to prime synthesis of said third set oligonucleotides therefrom, the 3′ end of the first set oligonucleotides are not complementary to the 5′ end of the third set oligonucleotides, and the 3′ end of the second set oligonucleotides are not complementary to the 5′ end of the first set oligonucleotides.

In a preferred embodiment of the above paragraph, each X at a given position in the formula is the same in one, two or three of the subsets of the 256 zinc finger domains and can be from a known zinc finger framework. The codon usage in the oligonucleotides can be also be optimized for any desired organism for which such information is available, such as, but not limited to human, mouse, cereal plants, tomato, corn, rice, and E. coli. Further, any of the above sets can be provided in kit form and include other components that enable one to readily practice the methods of the invention.

Any of the oligonucleotide sets of the invention can be provided as kits for preparing ZFPs. Such kits can include buffers, controls, instructions and the like useful in preparing ZFPs by the modular assembly method of the invention.

Another aspect of the invention relates to single-stranded or double-stranded oligonucleotide encoding a zinc finger domain for an artificial ZFP, said oligonucleotide being from about 84 to about 130 bases and comprising a nucleotide sequence encoding a zinc finger domain independently represented by the formula

-X₃-Cys-X₂₋₄-Cys-X₅-Z⁻¹-X-Z²-Z³-X₂-Z⁶-His-X₃₋₅-His-X₄-

and, optionally, a linker of from 0 to 10 amino acid residues; wherein X is any amino acid and Xₙ represents the number of occurrences of X in the polypeptide chain; Z⁻¹ is arginine, glutamine, threonine, methionine or glutamic acid; Z² is serine, asparagine, threonine or aspartic acid; Z³ is histidine, asparagine, serine or aspartic acid; and Z⁶ is arginine, glutamine, threonine, tyrosine, leucine or glutamic acid. The X positions can be the framework of a Sp1C or Zif268 zinc finger domain. The nucleotide sequences can also be selected to provide optimal codon usage in a desired organism.

### BRIEF DESCRIPTION OF THE DRAWINGS

FIG. 1 is a schematic diagram showing the binding of one unit of a zinc finger domain to a 4 base pair DNA target site. The residues at positions −1, 2, 3 and 6 each independently contact one base. Position 1 is the start of the α-helix in a zinc finger domain.

FIG. 2 shows known and possible base interactions with amino acids. Interactions similar to those shown between guanine and histidine can be made with other amino acids that donate hydrogen bonds (serine and lysine). Interactions similar to those shown between thymidine and threonine can be made with other hydrophobic amino acids. Interactions similar to those shown and between thymidine and threonine/serine can be made with other amino acids that donate hydrogen bonds.

FIG. 3 shows the recognition of the 4^{th} base in a 4 base pair DNA target sequence by amino acids at position 2 of a zinc finger domain.

FIG. 4 is a schematic diagram of a wild type transposase (left) and engineered (artificial) transposase (right).

FIG. 5 is a schematic diagram depicting methods for performing site-specific genomic knock-outs and knock-ins using ZFPs.

FIG. 6 is a schematic diagram showing molecular switch methods for manipulating translocation of ZFPs into the nucleus using small molecules.

FIG. 7 is a schematic diagram showing the design of a ZFP targeting the AL1 binding site in Tomato Golden Mosaic Virus. The AL1 target site is SEQ ID NO: 14; Zif1 is SEQ ID NO: 15; Zif2 is SEQ ID NO: 16; and Zif3 is SEQ ID NO:17. Zif is=zinc finger domain.

FIG. 8 is depicts bar graphs showing DNA base selectivities of the Asp (left) and Gly (right) mutants at position 2 of the zinc finger domain shown.

FIG. 9 is a schematic diagram showing transposition of a kanamycin resistance gene (Kan^{R}) from a donor vector into a target sequence in an acceptor vector.

FIG. 10 is a schematic diagram illustrating assembly of 6-finger ZFPs.

### DETAILED DESCRIPTION OF THE INVENTION

I. Recognition Code and Design Methods

The present invention provides a context-independent recognition code by which zinc finger domains contact bases on a target polynucleotide sequence. This recognition code allows the design of ZFPs which can target any desired nucleotide sequence with high affinity. Previous recognition data is largely context-dependent and was generated by the use of phage display methods and targeting of three base pair sequences (Beeril et al., Biochemistry 95:14631, 1998; Wu et al. Biochemistry 92:345, 1995; Berg et al., Nature Struct. Biol. 3:941, 1996). Berg et al. used three zinc finger domains in which the first and second were same, and the third was different than the first and second. Barbas used three zinc finger domains (Zif268) in which each of the three fingers was different. The present invention relates, inter alia, to an exactly repeating finger/frame block in that the same frame, and optionally the same finger region, is repeated. One advantage of repeating the same frame is that each zinc finger domain recognizes 4 base pairs regularly, which results in higher affinity targeting for ZFPs comprising multiple zinc finger domains, particularly when more than three domains (e.g., 4, 5, 6, 7, 8, 9, 10, 11, 12 domains or more, even up to 30 domains) are present.

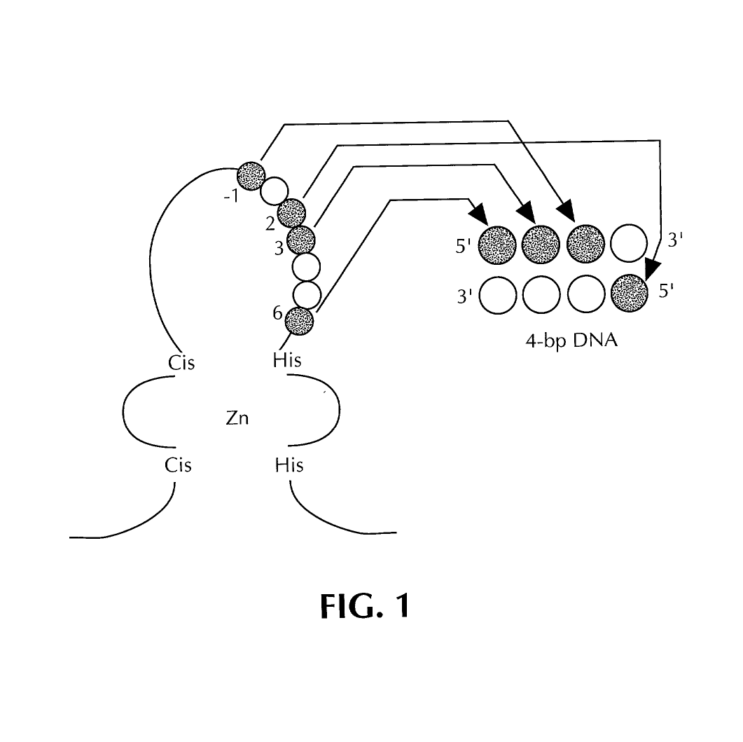

Four nucleic acid-contacting residues in zinc finger domains are primarily responsible for determining specificity and affinity and occur in the same position relative to the first consensus histidine and second consensus cysteine. The first residue is seven residues to the N-terminal side of the first consensus histidine and six residues to the C-terminal side of the second consensus cysteine. This is hereinafter referred to as the “−1 position.” The other three amino acids are two, three and six residues removed from the C-terminus of the residue at position −1, and are referred to as the “2 position”, “3 position” and “6 position”, respectively. These positions are interchangeably referred to as the Z⁻¹, Z², Z³ and Z⁶ positions. These amino acid residues are referred to as the base-contacting amino acids. Position 1 is the start of the α-helix in a zinc finger domain. The location of amino acid positions −1, 2, 3 and 6 in a zinc finger domain, and the bases they contact in a 4 base pair DNA target sequence, are shown schematically in FIG. 1.

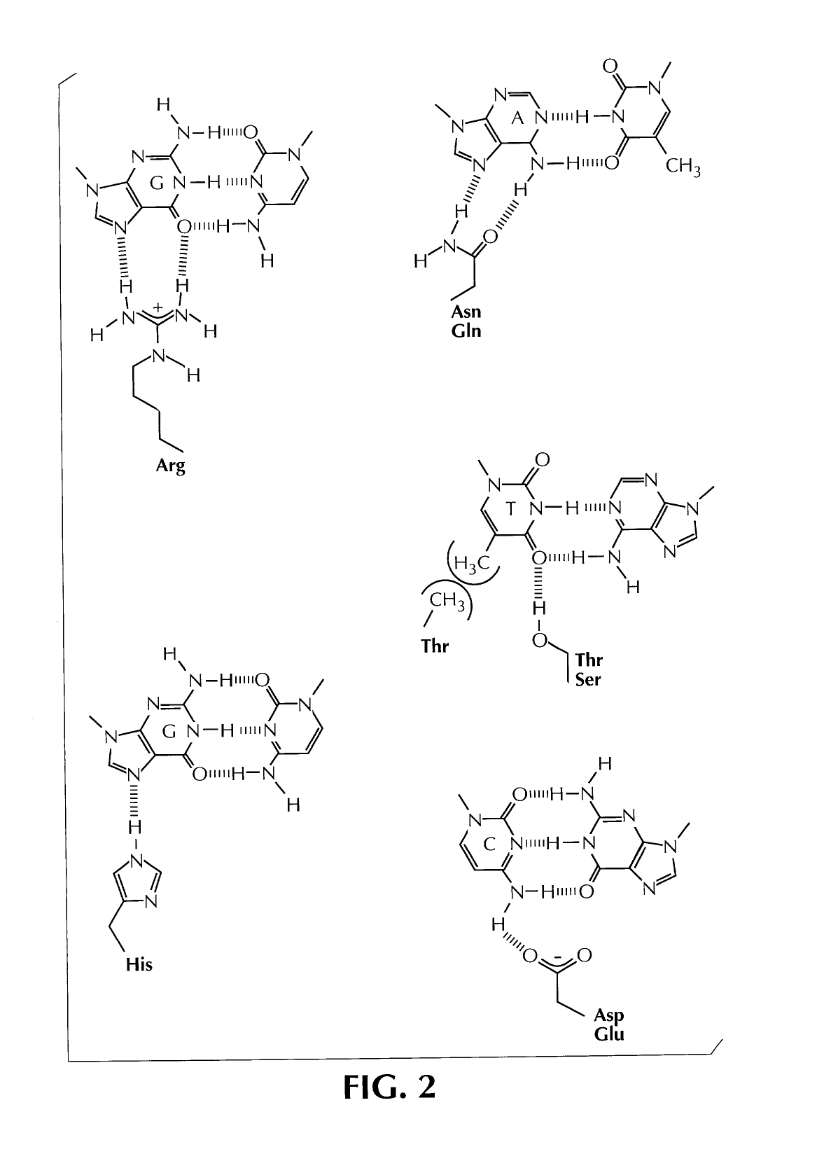

A zinc finger-nucleic acid recognition code is shown in Table 1 and is based on known and possible base-amino acid interactions (FIG. 2). Some interactions listed in FIG. 2 are also identified in different proteins such as H-T-4 protein, cro and the λ repressor. For recognition of the first and third DNA bases in a four base pair region, amino acids containing longer side chains were chosen. For recognition of the second and fourth bases, amino acids containing shorter side chains were chosen. For example, in the case of guanine base recognition, arginine was chosen as an amino acid at positions −1 and 6, histidine was chosen as an amino acid at position 3 and serine was chosen as an amino acid at position 2. In all of the amino acids shown in Table 1, there is stable interaction with specific DNA bases by hydrogen bonding. In the case of thymidine base recognition, amino acid having hydrophobic side chains were also chosen (i.e., leucine for first thymidine base and methionine for third thymidine base). Other DNA base-amino acid interaction is possible; however, amino acids with the highest affinity were chosen. For example, although lysine binds to guanine, arginine was chosen because of additional hydrogen bonding.

TABLE 1

| | 1^{st} base | 2^{nd} base | 3^{rd} base | 4^{th} base |
|---|---|---|---|---|
| G | Arg | His | Arg | Ser |
| A | Gln | Asn | Gln | Asn |
| T | Thr, Tyr, Leu | Ser | Thr, Met | Thr |
| C | Glu | Asp | Glu | Asp |
| | Position 6 | Position 3 | Position −1 | Position 2 |

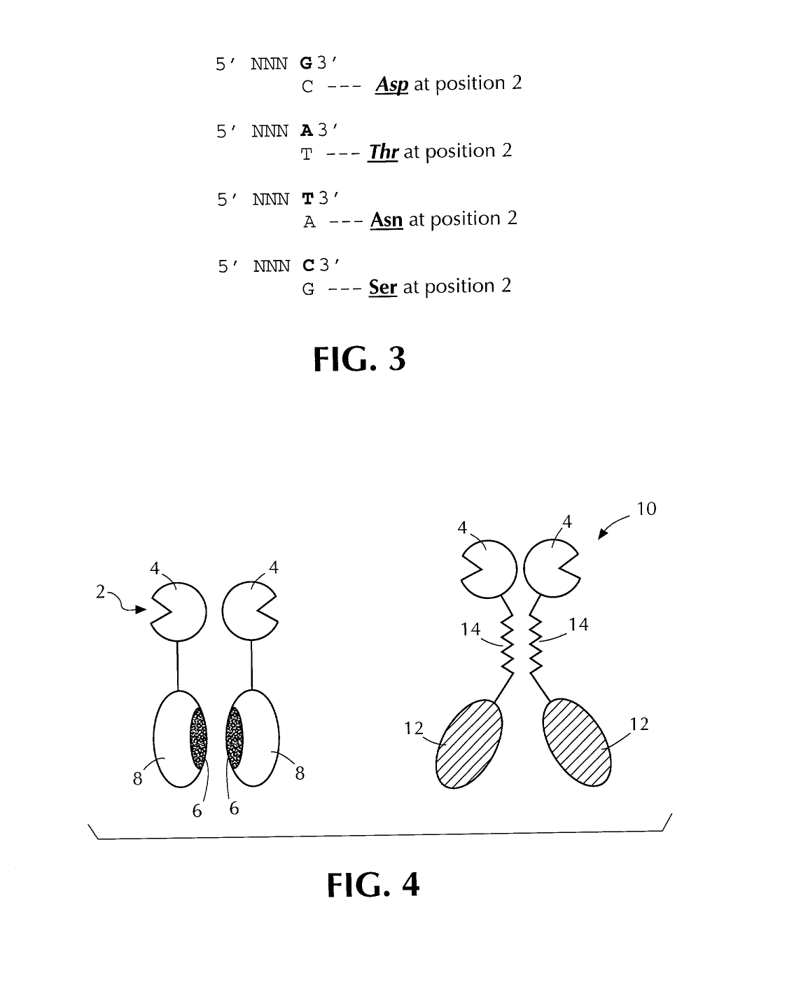

The recognition of the fourth base in a 4 base pair DNA sequence (1^{st} base of a neighboring 3′ triplet DNA) by amino acids at position 2 is shown in FIG. 3. Asp, Thr, Asn and Ser at position 2 of a zinc finger domain preferentially bind to C, T, A, and G, respectively. The fourth base is in the anti-sense nucleic acid strand.

In Table 1 (and for each 4 base-pair portion of a target sequence), the bases are always provided in 5′ to 3′ order. The fourth base listed in the table, however, is always the complement of the fourth base provided in the target sequence. For example, if the target sequence is written as ATCC, then it means a sense strand target sequence of 5′-ATCC-3′ and an antisense strand of 3′-TAGG-5′. Thus, when the sense strand sequence ATCC is translated to amino acids from the table above, the first base of A means there is glutamine at position 6, the second base of T means there is serine at position 3 and the third base of C means there is glutamic acid at position −1. However, with the fourth base written as C, it means that it is the complement of C, i.e., G, which is found in the table and used to identify the amino acid of position 2. In this case, the amino acid at position two is serine.

The present invention also includes a preferred recognition code table, where Z⁶ is threonine if the first base is T and where Z⁻¹ is threonine if the third base is T. In addition, the invention includes a recognition code table enlarged to generally provide additional conservative amino acids for those present in the recognition code of Table 1. This broader recognition code is below provided in Table 2. In Table 2, the order of amino acids listed in each box represents, from left to right, the most preferred to least preferred amino acid at that position.

TABLE 2

| | 1^{st} base | 2^{nd} base | 3^{rd} base | 4^{th} base |
|---|---|---|---|---|
| G | Arg, Lys | His, Lys | Arg, Lys | Ser, Arg |
| A | Gln, Asn | Asn, Gln | Gln, Asn | Asn, Gln |
| T | Thr, Tyr, Leu, | Ser, Ala, Met | Thr, Met, Leu, | Thr, Val, Ala |
| | Ile, Met | | Ile | |
| C | Glu, Asp | Asp, Glu | Glu, Asp | Asp, Glu |
| | Position 6 | Position 3 | Position −1 | Position 2 |

The present invention makes it possible to quickly design ZFPs targeting all possible DNA base pairs by choosing 4 amino acids per zinc finger domain from the recognition code table and by combining each domain. Such a complete recognition code table does not currently exist. By using the recognition code of the present invention, it is not necessary to select all possible mutants by repeating time-consuming selection like in a phage display system. By including amino acids at position 2 in the design, it becomes feasible to make ZHPs with higher affinity and DNA sequence selectivity because four, instead of three, base pairs are targeted. Current approaches to designing ZFPs using phage target or consider only three base pairs. The present invention provides ZFPs with increases in both specificity and binding affinity.

Thus the present invention provides methods of designing zinc finger domains. A single zinc finger domain represented by the formula

-X₃-Cys-X₂₋₄-Cys-X₅-Z⁻¹-X-Z²-Z³-X₂-Z⁶-His-X₃₋₅-His-X₄-,

wherein X is any amino acid and Xₙ represents the number of occurrences of X in the polypeptide chain, can be designed by identifying a target nucleic acid sequence of four bases; determining the identity of each X, and determining the identity of the amino acids at positions Z⁻¹, Z², Z³ and Z⁶ in the domain using the recognition code of Table 1, Table 2 or the preferred embodiment of Table 1. Once a zinc finger domain is designed, that domain can be included as all or part of any polypeptide chain. For example, the designed domain can be a single finger of a multi-fingered ZFP. That designed domain could also occur more than one time in a ZFP, and be contiguous with or separated from the other zinc finger domains designed in accordance with the invention. The zinc finger domain designed in accordance with the invention can also be included as a domain in non-ZFP proteins or as a domain in fusion proteins of any type. Preferably the designed domain is used to prepare a ZFP comprising that domain.

The framework determined by the identity of X can be a known zinc finger framework, a consensus framework or an alteration of any one of these frameworks provided that the altered framework maintains the overall structure of zinc finger domain. Preferred frameworks are those from Sp1C and Zif268. A more preferred framework is domain 2 form Sp1C.

The proteins containing the designed zinc finger domain can be prepared either synthetically or recombinantly, preferably recombinantly, using any of the multitude of techniques well-known in the art. When the proteins are prepared recombinantly, e.g., via a DNA encoding the ZFP, the codon usage can be optimized for high expression in the organism in which that ZFP is to be expressed. Such organisms include bacteria, fungi, yeast, animals, insects and plants. More specifically the organisms, include but are not limited to, human, mouse, E. coli, cereal plants, rice, tomato and corn.

To design a multi-domained (i.e., a multi-fingered) ZFP, the above method for designing a single domain can be followed, especially if the domains are not contiguous. However, for ZFPs with multiple contiguous domains (or domains separated by linkers as provided herein) for target sequences greater than 4 bases pairs, it has been discovered that ZFPs designed by dividing the target sequence into overlapping 4 base pair segments provides a context-independent zinc finger recognition code from which to produce ZFPs, and typically, ZFPs with high binding affinity, especially when there are more than three zinc finger domains in the ZFP.

In this method, the target sequence has a length of 3N+1 base pairs, wherein N is the number of overlapping 4 base pair segments in the target and is determined by dividing the target sequence into overlapping 4 base pair segments, where the fourth base of each segment, up to the N−1 segment, is the first base of the immediately following segment. The remainder of the design method for each 4 base pair segment follows that of a single domain with respect to determining the identities of each X, Z⁻¹, Z², Z³ and Z⁶. This method is useful for designing ZFPs having from 3 to 15 domains (i.e., N is any number from 3 to 15), and more preferably from 3 to 12 domains, from 3 to 9 domains or from 3 to 6 domains. Since ZFPs with more than 40 domains are known in the art, if desired, N can range to at least 40, if not more.

The zinc finger domains designed in accordance with this invention are either covalently joined directly one to another or can be separated by a linker region of from 1-10 amino acids. The linker amino acids can provide flexibility or some degree of structural rigidity. The choice of linker can be, but is not necessarily, dictated by the desired affinity of the ZFP for its cognate target sequence. It is within the skill of the art to test and optimize various linker sequences to improve the binding affinity of the ZFP for its cognate target sequence. Methods of measuring binding affinity between ZFPs and their targets are well known. Typically gel shift assays are used. In one embodiment, the amino acid linker is preferably be flexible to allow each three finger domain to independently bind to its target sequence and avoid steric hindrance of each other's binding.

The recognition code table has four amino acid positions and there are four different bases that each amino acid could target. The total number of different four base pair targets is represented by 4⁴ or 256. Using the preferred choices from the recognition code of Table 1, the combinations of amino acids for positions −1, 2, 3 and 6 in a zinc finger domain are provided in Table 3 for all possible 4 base pair target sequences.

TABLE 3
256 Zinc-Finger Domains for Preferred Recognition Code of Table 1

| No. | 4-bp Target | Position-1 | Position 2 | Position 3 | Position 6 | Group |
|---|---|---|---|---|---|---|
| 1 | GGGG | Arg | Asp | His | Arg | |
| 2 | GGGA | Arg | Thr | His | Arg | |
| 3 | GGGT | Arg | Asn | His | Arg | |
| 4 | GGGC | Arg | Ser | His | Arg | |
| 5 | GGAG | Gin | Asp | His | Arg | 1 |
| 6 | GGAA | Gin | Thr | His | Arg | |
| 7 | GGAT | Gin | Asn | His | Arg | 1 |
| 8 | GGAC | Gin | Ser | His | Arg | |
| 9 | GGTG | Thr | Asp | His | Arg | |
| 10 | GGTA | Thr | Thr | His | Arg | 2 |
| 11 | GGTT | Thr | Asn | His | Arg | 2 |
| 12 | GGTC | Thr | Ser | His | Arg | |
| 13 | GGCG | Glu | Asp | His | Arg | 3 |
| 14 | GGCA | Gin | Thr | His | Mg | 3 |
| 15 | GOCT | Gin | Asn | His | Mg | 3 |
| 16 | GGCC | Glu | Ser | His | Mg | 3 |
| 17 | GAGG | Mg | Asp | Asn | Mg | |
| 18 | GAGA | Mg | Thr | Asn | Mg | 4 |
| 19 | GAGT | Mg | Asn | Asn | Mg | 4 |
| 20 | GAGC | Mg | Ser | Asn | Mg | |
| 21 | GAAG | Gin | Asp | Asn | Mg | |
| 22 | GAAA | Gin | Thr | Asn | Mg | |
| 23 | GAAT | Gin | Asn | Asn | Mg | |
| 24 | GAAC | Gin | Ser | Asn | Mg | |
| 25 | GATG | Thr | Asp | Asn | Mg | 5 |
| 26 | GATA | Thr | Thr | Asn | Mg | |
| 27 | GATT | Thr | Asn | Asn | Mg | 5 |
| 28 | GATC | Thr | Ser | Asn | Mg | |
| 29 | GACG | Glu | Asp | Asn | Mg | 6 |
| 30 | GACA | Glu | Thr | Asn | Mg | 6 |
| 31 | GACT | Glu | Asn | Asn | Mg | 6 |
| 32 | GACC | Glu | Ser | Asn | Mg | |
| 33 | GTGG | Mg | Asp | Ser | Mg | |
| 34 | GTGA | Mg | Thr | Ser | Mg | 7 |
| 35 | GTGT | Mg | Asn | Ser | Mg | 7 |
| 36 | GTGC | Mg | Ser | Ser | Mg | |
| 37 | GTAG | Gin | Asp | Ser | Mg | 8 |
| 38 | GTAA | Gin | Thr | Ser | Mg | |
| 39 | GTAT | Gin | Asn | Ser | Mg | 8 |
| 40 | GTAC | Gin | Ser | Ser | Mg | |
| 41 | GTTG | Thr | Asp | Ser | Mg | 9 |
| 42 | GTTA | Thr | Thr | Ser | Mg | 9 |
| 43 | GTTT | Thr | Asn | Ser | Mg | |
| 44 | GTTC | Thr | Ser | Ser | Mg | |
| 45 | GTGG | Glu | Asp | Ser | Mg | |
| 46 | GTCA | Glu | Thr | Ser | Mg | |
| 47 | GTCT | Gin | Asn | Ser | Mg | |
| 48 | GTCC | Glu | Ser | Ser | Mg | |
| 49 | GCGG | Mg | Asp | Asp | Mg | |
| 50 | GCGA | Mg | Thr | Asp | Mg | |
| 51 | GCGT | Arg | Asn | Asp | Arg | |
| 52 | GCGC | Arg | Ser | Asp | Arg | |
| 53 | GCAG | Gin | Asp | Asp | Arg | |
| 54 | GCAA | Gin | Thr | Asp | Arg | |
| 55 | GCAT | Gin | Asn | Asp | Arg | |
| 56 | GCAC | Gin | Ser | Asp | Mg | |
| 57 | GCTG | Thr | Asp | Asp | Mg | 10 |
| 58 | GCTA | Thr | Thr | Asp | Mg | 10 |
| 59 | GCTT | Thr | Asn | Asp | Mg | 10 |
| 60 | GCTC | Thr | Ser | Asp | Mg | 10 |
| 61 | GCCG | Glu | Asp | Asp | Mg | 11 |
| 62 | GCCA | Glu | Thr | Asp | Mg | 11 |
| 63 | GCCT | GLu | Asn | Asp | Mg | 11 |
| 64 | GCCC | Glu | Ser | Asp | Mg | |
| 65 | AGGG | Mg | Asp | His | Gin | |
| 66 | AGGA | Mg | Thr | His | Gin | 12 |
| 67 | AGGT | Mg | Asn | His | Gin | 12 |
| 68 | AGGC | Mg | Ser | His | Gin | |
| 69 | AGAG | Gin | Asp | His | Gin | 12 |
| 70 | AGAA | Gin | Thr | His | Gin | |
| 71 | AGAT | Gin | Asn | His | Gin | 12 |
| 72 | AGAC | Gin | Ser | His | Gin | |
| 73 | AGTG | Thr | Asp | His | Gin | 12 |
| 74 | AGTA | Thr | Thr | His | Gin | 12 |
| 75 | AGTT | Thr | Asn | His | Gin | 12 |
| 76 | AGTC | Thr | Ser | His | Gin | 12 |
| 77 | AGCG | Glu | Asp | His | Gin | 12 |
| 78 | AGCA | Glu | Thr | His | Gin | 12 |
| 79 | AGCT | Glu | Asn | His | Gin | 12 |
| 80 | AGCC | Glu | Ser | His | Gin | 12 |
| 81 | AAGG | Arg | Asp | Asn | Gin | |
| 82 | AAGA | Arg | Thr | Asn | Gin | 13 |
| 83 | AAGT | Arg | Asn | Asn | Gin | 13 |
| 84 | AAGC | Arg | Ser | Asn | Gin | |
| 85 | AAAG | Gin | Asp | Asn | Gin | 13 |
| 86 | AAAA | Gin | Thr | Asn | Gin | 13 |
| 87 | AAAT | Gin | Asn | Asn | Gin | 13 |
| 88 | AAAC | Gin | Ser | Asn | Gin | |
| 89 | AATG | Thr | Asp | Asn | Gin | 13 |
| 90 | AATA | Thr | Thr | Asn | Gin | 13 |
| 91 | AATT | Tbr | Asn | Asn | Gin | 13 |
| 92 | AATC | Thr | Ser | Asn | Gin | 13 |
| 93 | AACG | Glu | Asp | Mn | Gin | 13 |
| 94 | AACA | Glu | Thr | Asn | Gin | 13 |
| 95 | AAGT | Glu | Asn | Asn | Gin | |
| 96 | AACC | Glu | Ser | Asn | Gin | 13 |
| 97 | ATGG | Arg | Asp | Ser | Gin | 14 |
| 98 | ATGA | Arg | Thr | Ser | Gin | 14 |
| 99 | ATGT | Arg | Asn | Ser | Gin | 14 |
| 100 | ATGC | Arg | Ser | Ser | Gin | |
| 101 | ATAG | Gin | Asp | Ser | Gin | 14 |
| 102 | ATAA | Gin | Thr | Ser | Gin | 14 |
| 103 | ATAT | Gin | Asn | Ser | Gin | 14 |
| 104 | ATAC | Gin | Ser | Ser | Gin | |
| 105 | ATTG | Thr | Asp | Ser | Gin | 14 |
| 106 | ATTA | Thr | Thr | Ser | Gin | 14 |
| 107 | ATTT | Thr | Asn | Ser | Gin | 14 |
| 108 | ATTC | Thr | Ser | Ser | Gin | 14 |
| 109 | ATCG | Glu | Asp | Ser | Gin | 14 |
| 110 | ATCA | Glu | Thr | Ser | Gin | 14 |
| 111 | ATCT | Glu | Asn | Ser | Gin | 14 |
| 112 | ATCC | Glu | Ser | Ser | Gin | 14 |
| 113 | ACGG | Mg | Asp | Asp | Gin | 15 |
| 114 | ACGA | Mg | Thr | Asp | Gin | |
| 115 | ACGT | Mg | Asn | Asp | Gin | 15 |
| 116 | ACGC | Mg | Ser | Asp | Gin | 15 |
| 117 | ACAG | Gin | Asp | Asp | Gin | 15 |
| 118 | ACAA | Gin | Thr | Asp | Gin | 15 |
| 119 | ACAT | Gin | Asn | Asp | Gin | 15 |
| 120 | ACAC | Gin | Ser | Asp | Gin | 15 |
| 121 | ACTG | Thr | Asp | Asp | Gin | 15 |
| 122 | ACTA | Thr | Thr | Asp | Gin | 15 |
| 123 | ACTT | Thr | Asn | Asp | Gin | 15 |
| 124 | ACTC | Thr | Ser | Asp | Gin | 15 |
| 125 | ACCG | Glu | Asp | Asp | Gin | 15 |
| 126 | ACCA | Glu | Thr | Asp | Gin | 15 |
| 127 | ACCT | Glu | Asn | Asp | Gin | 15 |
| 128 | ACCC | Glu | Ser | Asp | Gin | 15 |
| 129 | TGGG | Mg | Asp | His | Thr | |
| 130 | TGGA | Mg | Thr | His | Thr | |
| 131 | TGGT | Mg | Asn | His | Thr | |
| 132 | TGGC | Arg | Ser | His | Thr | |
| 133 | TGAG | Gin | Asp | His | Thr | 16 |
| 134 | TGAA | Gin | Thr | His | Thr | 16 |
| 135 | TGAT | Gin | Asn | His | Thr | 16 |
| 136 | TGAC | Gin | Ser | His | Thr | |
| 137 | TGTG | Thr | Asp | His | Thr | 16 |
| 138 | TGTA | Thr | Thr | His | Thr | 16 |
| 139 | TGTT | Thr | Asn | His | Thr | 16 |
| 140 | TGTC | Thr | Ser | His | Thr | 16 |
| 141 | TGCG | Glu | Asp | His | Thr | 16 |
| 142 | TGCA | Glu | Thr | His | Thr | 16 |
| 143 | TGCT | Glu | Asn | His | Thr | 16 |
| 144 | TGCC | Glu | Ser | His | Thr | 16 |
| 145 | TAGG | Arg | Asp | Asn | Thr | 17 |
| 146 | TAGA | Arg | Thr | Asn | Thr | 17 |
| 147 | TAGT | Arg | Asn | Asn | Thr | 17 |
| 148 | TAGC | Arg | Ser | Asn | Thr | |
| 149 | TAAG | Gin | Asp | Asn | Thr | |
| 150 | TAAA | Gin | Thr | Asn | Thr | |
| 151 | TAAT | Gin | Asn | Asn | Thr | |
| 152 | TAAC | Gin | Ser | Asn | Thr | |
| 153 | TATG | Thr | Asp | Asn | Thr | 17 |
| 154 | TATA | Thr | Thr | Asn | Thr | 17 |
| 155 | TATT | Thr | Asn | Asn | Thr | 17 |
| 156 | TATC | Thr | Ser | Asn | Thr | 17 |
| 157 | TACG | Glu | Asp | Asn | Thr | 17 |
| 158 | TACA | Glu | Thr | Asn | Thr | 17 |
| 159 | TACT | Glu | Asn | Asn | Thr | 17 |
| 160 | TACC | Glu | Ser | Asn | Thr | 17 |
| 161 | TTGG | Arg | Asp | Ser | Thr | 18 |
| 162 | TTGA | Arg | Thr | Ser | Thr | 18 |
| 163 | TTGT | Arg | Asn | Ser | Thr | 18 |
| 164 | TTGC | Arg | Ser | Ser | Thr | |
| 165 | TTAG | Gin | Asp | Ser | Thr | 18 |
| 166 | TTAA | Gin | Thr | Ser | Thr | 18 |
| 167 | TTAT | Gin | Asn | Ser | Thr | 18 |
| 168 | TTAC | Gin | Ser | Ser | Thr | |
| 169 | TTTG | Thr | Asp | Ser | Thr | 18 |
| 170 | TTTA | Thr | Thr | Ser | Thr | 18 |
| 171 | TTTT | Thr | Asn | Ser | Thr | 18 |
| 172 | TTTC | Thr | Ser | Ser | Thr | 18 |
| 173 | TTCG | Glu | Asp | Ser | Thr | 18 |
| 174 | TTCA | Glu | Thr | Ser | Thr | 18 |
| 175 | TTCT | Glu | Asn | Ser | Thr | 18 |
| 176 | TTCC | Glu | Ser | Ser | Thr | 18 |
| 177 | TCGG | Arg | Asp | Asp | Thr | |
| 178 | TCGA | Arg | Thr | Asp | Thr | |
| 179 | TCGT | Arg | Asn | Asp | Thr | |
| 180 | TCGC | Arg | Ser | Asp | Thr | |
| 181 | TCAG | Gin | Asp | Asp | Thr | 19 |
| 182 | TCAA | Gin | Thr | Asp | Thr | 19 |
| 183 | TCAT | Gin | Asn | Asp | Thr | 19 |
| 184 | TCAC | Gin | Ser | Asp | Thr | 19 |
| 185 | TCTG | Thr | Asp | Asp | Thr | 19 |
| 186 | TCTA | Thr | Thr | Asp | Thr | 19 |
| 187 | TCTT | Thr | Asn | Asp | Thr | 19 |
| 188 | TCTC | Thr | Ser | Asp | Thr | 19 |
| 189 | TCCG | Glu | Asp | Asp | Thr | 19 |
| 190 | TCCA | Glu | Thr | Asp | Thr | 19 |
| 191 | TCCT | Glu | Asn | Asp | Thr | 19 |
| 192 | TCCC | Glu | Ser | Asp | Thr | 19 |
| 193 | CGGG | Arg | Asp | His | Glu | 20 |
| 194 | CGGA | Arg | Thr | His | Glu | |
| 195 | CGGT | Arg | Asn | His | Glu | 20 |
| 196 | CGGC | Arg | Ser | His | Glu | |
| 197 | CGAG | Gin | Asp | His | Glu | 20 |
| 198 | CGAA | Gin | Thr | His | Glu | 20 |
| 199 | CGAT | Gin | Asn | His | Glu | 20 |
| 200 | CGAC | Gin | Ser | His | Glu | |
| 201 | CGTG | Thr | Asp | His | Glu | 20 |
| 202 | CGTA | Thr | Thr | His | Glu | 20 |
| 203 | CGTT | Thr | Asn | His | Glu | 20 |
| 204 | CGTC | Thr | Ser | His | Glu | 20 |
| 205 | CGCG | Glu | Asp | His | Glu | 20 |
| 206 | CGCA | Glu | Thr | His | Glu | 20 |
| 207 | CGCT | Glu | Asn | His | Glu | 20 |
| 208 | CGCC | Glu | Ser | His | Glu | 20 |
| 209 | CAGG | Arg | Asp | Asn | Glu | |
| 210 | CAGA | Arg | Thr | Asn | Glu | 21 |
| 211 | CAGT | Arg | Asn | Asn | Glu | 21 |
| 212 | CAGC | Arg | Ser | Asn | Glu | |
| 213 | CAAG | Gin | Asp | Asn | Glu | 21 |
| 214 | CAAA | Gin | Thr | Asn | Glu | 21 |
| 215 | CAAT | Gin | Asn | Asn | Glu | 21 |
| 216 | CAAC | Gin | Ser | Asn | Glu | |
| 217 | GATG | Thr | Asp | Asn | Glu | 21 |
| 218 | CATA | Thr | Ttir | Asn | Glu | 21 |
| 219 | CATT | Thr | Asn | Asn | Glu | 21 |
| 220 | CATC | Thr | Ser | Asn | Glu | 21 |
| 221 | CACG | Glu | Asp | Asn | Glu | 21 |
| 222 | CACA | Glu | Thr | Asn | Gin | 21 |
| 223 | CACT | Glu | Asn | Asn | Glu | 21 |
| 224 | CACC | Glu | Ser | Asn | Glu | 21 |
| 225 | CTGG | Arg | Asp | Ser | Glu | 22 |
| 226 | CTGA | Arg | Thr | Ser | Glu | |
| 227 | CTGT | Arg | Asn | Ser | Glu | 22 |
| 228 | CTGC | Arg | Ser | Ser | Glu | |
| 229 | CTAG | Gin | Asp | Ser | Glu | 22 |
| 230 | CTAA | Gin | Thr | Ser | GLu | 22 |
| 231 | CTAT | Gin | Asn | Ser | Glu | 22 |
| 232 | CTAC | Gin | Ser | Ser | Glu | |
| 233 | CTTG | Thr | Asp | Ser | Glu | 22 |
| 234 | CTTA | Thr | Thr | Ser | Glu | 22 |
| 235 | CTTT | Thr | Asn | Ser | Glu | 22 |
| 236 | CTTC | Thr | Ser | Ser | Glu | 22 |
| 237 | CTCG | Glu | Asp | Ser | Glu | 22 |
| 238 | CTCA | Glu | Thr | Ser | Glu | 22 |
| 239 | CTCT | Glu | Asn | Ser | Glu | 22 |
| 240 | CTCC | GLu | Ser | Ser | Glu | |
| 241 | CCGG | Arg | Asp | Asp | Glu | |
| 242 | CCGA | Arg | Thr | Asp | Glu | |
| 243 | CCGT | Arg | Asn | Asp | Glu | |
| 244 | CCGC | Arg | Ser | Asp | Glu | |
| 245 | CCAG | Gin | Asp | Asp | Glu | 23 |
| 246 | CCAA | Gin | Thr | Asp | Glu | 23 |
| 247 | CCAT | Gin | Asn | Asp | Glu | 23 |
| 248 | CCAC | Gin | Ser | Asp | Glu | 23 |
| 249 | CCTG | Thr | Asp | Asp | Glu | 23 |
| 250 | CCTA | Thr | Thr | Asp | Glu | 23 |
| 251 | CCTT | Thr | Asn | Asp | Glu | 23 |
| 252 | CCTC | Thr | Ser | Asp | Glu | 23 |
| 253 | CCCG | Glu | Asp | Asp | Glu | 23 |
| 254 | CCCA | Glu | Thr | Asp | Glu | 23 |
| 255 | CCCT | Glu | Asn | Asp | Glu | 23 |
| 256 | CCCC | Glu | Ser | Asp | Glu | 23 |

“Specifically binds” means and includes reference to binding of a zinc-finger-protein-nucleic-acid-binding domain to a specified nucleic acid target sequence to a detectably greater degree (e.g., at least 1.5-fold over background) than its binding to non-target nucleic acid sequences and to the substantial exclusion of non-target nucleic acids.

When a multi-finger ZFP binds to a polynucleotide duplex (e.g. DNA, RNA, peptide nucleic acid (PNA) or any hybrids thereof) its fingers typically line up along the polynucleotide duplex with a periodicity of about one finger per 3 bases of nucleotide sequence. The binding sites of individual zinc fingers (or subsites) typically span three to four bases, and subsites of adjacent fingers usually overlap by one base. Accordingly, a three-finger ZFP XYZ binds to the 10 base pair site abcdefghij (where these letters indicate one of the duplex DNA) with the subsite of finger X being ghij, finger Y being defg and finger Z being abcd. The present invention encompasses multi-fingered proteins in which at least three fingers differ from a wild type zinc fingers. It also includes multi-fingered protein in which the amino acid sequence in all the fingers have been changed, including those designed by combinatorial chemistry or other protein design and binding assays but which correspond to a ZFP from the recognition code of Table 1.

It is also possible to design a ZFP to bind to a targeted polynucleotide in which more than four bases have been altered. In this case, more than one finger of the binding protein is a altered. For example, in the 10 base sequence XXXdefgXXX, a three-finger binding protein could be designed in which fingers X and Z differ from the corresponding fingers in a wild type zinc finger, while finger Y will have the same polypeptide sequence as the corresponding finger in the wild type fingers which binds to the subsite defg. Binding proteins having more than three fingers can be also designed for base sequences of longer length. For example, a four finger-protein will optimally bind to a 13 base sequence, while a five-finger protein will optimally bind to a 16 base sequence. A multi-finger protein can also be designed in which some of the fingers are not involved in binding to the selected DNA. Slight variations are also possible in the spacing of the fingers and framework.

It has surprisingly been found that good binding can be obtained for ZFPs that target any contiguous 10 bases having at least three guanines (three Gs) in the first nine bases, excluding the last quadruplet of the target. It is also preferred that such targets have two or fewer cytosines.

II. Artificial ZFPs

The present invention also relates to isolated, artificial ZFPs for binding to target nucleic acid sequences.

By “zinc finger protein”, “zinc finger polypeptide” or “ZFP” is meant a polypeptide having DNA binding domains that are stabilized by zinc and designed in accordance with the present invention with the proviso that the proteins do not include those of SEQ ID NOS: 3-12 (Table 4) or any other ZFP having three or more of the zinc finger domains designed in accordance with the recognition code of Table 1, where those domains are joined with 0 to 10 amino acids. The individual DNA binding domains are typically referred to as “fingers,” such that a ZFP or peptide has at least one finger, more typically two fingers, more preferably three fingers, or even more preferably four or five fingers, to at least six or more fingers. Each finger binds three or four base pairs of DNA. A ZFP binds to a nucleic acid sequence called a target nucleic acid sequence. Each finger usually comprises an approximately 30 amino acid, zinc-chelating, DNA-binding subdomain. A representative motif of one class, the Cys₂-His₂ class, is -CYS-(X)₂₋₄-CYS-(X)₁₂-His-(X)₃₋₅-His, where X is any amino acid, and a single zinc finger of this class consists of an alpha helix containing the two invariant histidine residues and the two cysteine residues of a single beta turn (see, e.g., Berg et al., Science 271:1081-1085 (1996)) bind a zinc cation.

The ZFPs of the invention include any ZFP having one or more combination of amino acids for positions −1, 2, 3 and 6 as provided by the recognition code in Table 1 (provided that the ZFP is not in the prior art). The 256 4-base pair target sequences of the ZFPs and the corresponding amino acids for positions −1, 2, 3 and 6 are provided in Table 3 for a preferred recognition code table of the invention (namely, that of Table 1, where if the first base is T, then Z⁶ is threonine; and if the third base is T, then Z⁻¹ is threonine). Preferably, a ZFP comprises from 3 to 15, 3 to 12, 3 to 9 or from 3 to 6 domains as well as three, four, five or six zinc finger domains but since ZFPs with up to 40 domains are known, the invention includes such ZFPs.

TABLE 4
ZFPs excluded from Claim 1

| SEQ | Search | | |
|---|---|---|---|
| ID. | Database | | |
| NO. | Identifier | General Description | Sequence |
| 3 | AN AAB07701 | Artificial (?) 5 finger | VPIPGKKKQHICHIQGCGKVYGQSSDLQRHL |
| | | protein to modulate | RWHTGERPFMCTWSYCGKRFTRSSNLQRHKR |
| | | gene expression | THTGEKKFACPECPKRFMRSDELSRHIKTHQ |
| | | | NKKDGGGSGKKKQHICHIQGCGKVYGTTSNL |
| | | | RRHLRWHTGERPFMCTWSYCGKRFTRSSNLQ |
| | | | RHKRTHTGEKKFACPECPKRFMRSDHLSRHI |
| | | | KTHQNKKGGS |
| 4 | AN AAB07699 | Artificial (?) 3 finger | VPIPGKKKQHICHIQGCGKVYGTTSNLRRHL |
| | | protein to modulate | RWHTGERPFMCTWSYCGKRFTRSSNLQRHKR |
| | | gene expression | THTGEKKFACPECPKRFMRSDHLSRHIKTHQ |
| | | | NKKGGS |
| 5 | RN 160082-26-8 | Synthetic zinc finger- | MEKLRNGSGDPGKKKQHACPECGKSFSQSSN |
| | | containing DNA- | LQRHQRTHTGEKPYKCPECGKSFSRSSHLQQ |
| | | binding reduced | HQRTHTGEKPYKCPECGKSFSRSDHLSRHQR |
| | | | THQNKK |
| 6 | RN 160082-24-6 | Synthetic zinc finger- | MEKLRNGSGDPGKKKQHACPECGKSFSQSSN |
| | | containing DNA- | LQRHQRTHTGEKPYKCPECGKSFSESSDLQR |
| | | binding reduced | HQRTHTGEKPYKCPECGKSFSRSDHLSRHQR |
| | | | THQNKK |
| 7 | RN 160082-20-2 | Synthetic zinc finger- | MEKLRNGSGDPGKKKQHACPECGKSFSQSSN |
| | | containing DNA- | LQRHQRTHTGEKPYKCPECGKSFSRSSHLQE |
| | | binding reduced | HQRTHTGEKPYKCPECGKSFSRSDHLSRHQR |
| | | | THQNKK |
| 8 | RN 160082-18-8 | Synthetic zinc finger- | MEKLRNGSGDPGKKKQHACPECGKSFSQSSN |
| | | containing DNA- | LQRHQRTHTGEKPYKCPECGKSFSQSSNLQR |
| | | binding reduced | HQRTHTGEKPYKCPECGKSFSRSDHLSRHQR |
| | | | THQNKK |
| 9 | RN 160082-17-7 | Synthetic zinc finger- | MEKLRNGSGDPGKKKQHACPECGKSFSQSSN |
| | | containing DNA- | LQRHQRTHTGEKPYKCPECGKSFSRSSNLQE |
| | | binding reduced | HQRTHTGEKPYKCPECGKSFSRSDHLSRHQR |
| | | | THQNKK |
| 10 | RN 160082-12-2 | Synthetic zinc finger- | MEKLRNGSGDPGKKKQHACPECGKSFSQSSN |
| | | containing DNA- | LQRHQRTHTGEKPYKCPECGKSFSQSSDLQR |
| | | binding reduced | HQRTHTGEKPYKCPECGKSFSRSDHLSRHQR |
| | | | THQNKK |
| 11 | RN 149024-80-6 | Human clone HKrT1 | MRLAKPKAGISRSSSQGKAYENKRKTGRQRE |
| | | zinc finger-containing | KWGMTIRFDSSFSRLRRSLDDKPYKCTECEK |
| | | reduced | SFSQSSTLFQHQKIHTGKKSHKCADCGKSFF |
| | | | QSSNLIQHRRIHTGEKPYKCDECGESFKQSS |
| | | | NLIQHQRIHTGEKPYQCDECGRCFSQSSHLI |
| | | | QHQRTHTGEKPYQCSECGKCFSQSSHLRQHM |
| | | | KVHKEEKPRKTRGKNIRVKTHLPSWKAGTEG |
| | | | SLWLVSVKYRAF |
| 12 | RN 147447-74-3 | Mouse clone pMLZ-4 | MSEEPLENAEKNPGSEEAFESGDQAERPWGD |
| | | zinc finger-containing | LTAEEWVSYPLQQVTDLLVHKEAHAGIRYHI |
| | | reduced | CSQCGKAFSQISDLNRHQKTHTGDRPYKCYE |
| | | | CGKGFSRSSHLIQHQRTHTGERPYDCNECGK |
| | | | SFGRSSHLIQHQTIHTGEKPHKCTECAKASA |
| | | | ASPHLIQHQRTHSGEKPYECEECGKSFSRSS |
| | | | HLAQHQRTHTGEKPYECHECGRGFSERSDLI |
| | | | KHYRVHTGERPYKCDECGKNFSQNSDLVRHR |
| | | | RAHTGEKPYHCNECGENFSRISHLVQHQRTH |
| | | | TGEKPYECTACGKSFSRSSHLITHQKIHTGE |
| | | | KPYECNECWRSFGERSDLIKHQRTHTGEKPY |
| | | | ECVQCGKGFTQSSNLITHQRVHTGEKPYECT |
| | | | ECDKSFSRSSALIKHKRVHTD |

In an embodiment of the invention, the isolated, artificial ZFPs designed for binding to a target nucleic acid sequence wherein the ZFPs comprising at least three zinc finger domains, each domain independently represented by the formula

-X₃-Cys-X₂₋₄-Cys-X₅-Z⁻¹-X-Z²-Z³-X₂-Z⁶-His-X₃₋₅-His-X₄-,

and the domains covalently joined to each other with a from 0 to 10 amino acid residues, wherein X is any amino acid and Xₙ represents the number of occurrences of X in the polypeptide chain, wherein Z⁻¹, Z², Z³, and Z⁶ are determined by the recognition code of Table 1 with the proviso that such proteins are not those provided by any one of SEQ ID NOS 3-12 (Table 4) or any other ZFP having three or more of the zinc finger domains designed in accordance with the recognition code of Table 1, where those domains are joined with 0 to 10 amino acids. As above, X represents a framework of a Cys₂His₂ zinc finger domain and can be a known zinc finger framework, a consensus framework, a framework obtained by varying the sequence any of these frameworks or any artificial framework. Preferably known frameworks are used to determine the identities of each X. The ZFPs of the invention comprise from 3 to 40 zinc finger domains, and preferably from 3 to 15 domains, 3 to 12 domains, 3 to 9 domains or 3 to 6 domains, as well as ZFPs with 3, 4, 5, 6, 7, 8 or 9 domains. In preferred embodiment the framework for determining X is that from Sp1C or Zif268. In one embodiment, the framework has the sequence of Sp1C domain 2, which sequence is -Pro-Tyr-Lys-Cys-Pro-Glu-Cys-Gly-Lys-Ser-Phe-Ser-Z⁻¹-Ser-Z²-Z³-Leu-Gln-Z⁶-His-Gln-Arg-Thr-His-Thr-Gly-Glu-Lys-(SEQ ID NO: 13).

Additionally preferred ZFPs are those wherein, independently or in any combination, Z⁻¹ is methionine in at least one of said zinc finger domains; Z⁻¹ is glutamic acid in at least one of said zinc finger domains; Z² is threonine in at least one of said zinc finger domains; Z² is serine in at least one of said zinc finger domains; Z² is asparagine in at least one of said zinc finger domains; Z⁶ is glutamic acid in at least one of said zinc finger domains; Z⁶ is threonine in at least one of said zinc finger domains; Z⁶ is tyrosine in at least one of said zinc finger domains; Z⁶ is leucine in at least one of said zinc finger domains and/or Z² is aspartic acid in at least one of said zinc finger domains, but Z⁻¹ is not arginine in the same domain.

The ZFPs of the invention also include the 23 groups of proteins as indicated in Table 3. Groups 1-11 represent proteins that bind the following classes of nucleotide target sequences GGAM, GGTW, GGCN, GAGW, GATM, GACD, GTGW, GTAM, GTTR, GCTN and GCCD, respectively, wherein D is G, A or T; M is G or T; R is G or A; W is A or T; and N is any nucleotide. The proteins of Groups 12-23 are generally represented by the formulas AGNN, AANN, ATNN, ACNN, TGNN, TANN, TTNN, TCNN, CGNN, CANN, CTNN, and CCNN, where N, however, does not represent any nucleotide but rather represents the nucleotides for the proteins designated as belonging to the group as set forth in Table 3.

Additional information relating to the ZFPs of the invention is provided throughout the specification.

Another aspect of the invention provides isolated nucleic acids encoding the ZFPs of the invention, expression vectors comprising those nucleic acids, and host cells transformed (by any method) with the expression vectors. Among other uses, such host cells can be used in a method of preparing a ZFP by culturing the host cell for a time and under conditions to express the ZFP; and recovering the ZFP. Such embodiments, i.e., nucleic acids, host cells, expression methods are included for any protein designed in accordance with the invention as well as the fusion proteins described below.

III. Fusion Proteins

In one embodiment of the invention, a ZFP fusion protein can comprise at least two DNA-binding domains, one of which is a zinc finger polypeptide, linked to the other domain via a flexible linker. The two domains can be the same or heterologous. In some embodiments of the invention, the ZFP can comprise two or more binding domains. In a preferred embodiment, at least one of these domains is a zinc finger and the other domain is another DNA binding protein such as a transcriptional activator.

The invention also includes any fusion protein with a ZFP of the invention fused to a protein of interest (POI) or a protein domain having an activity of interest. Such protein domains with a desired activity are also called effector domains.

In addition, the invention includes isolated fusion proteins comprising a ZFP of the invention fused to second domain (an effector domain) which is a transposase, integrase, recombinase, resolvase, invertase, protease, DNA methyltransferase, DNA demethylase, histone acetylase, histone deacetylase, nuclease, transcriptional repressor, transcriptional activator, single-stranded DNA binding protein, transcription factor recruiting protein nuclear-localization signal or cellular uptake signal. In an alternative embodiment, the second domain is a protein domain which exhibits transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, single-stranded DNA binding activity, transcription factor recruiting activity, or cellular uptake signaling activity.

Additional fusion proteins of the invention include a ZFP of the invention fused to a protein domain capable of specifically binding to a binding moiety of a divalent ligand which can be taken up by the cell. Such cellular uptake can be by any mechanism including, but not limited to, active transport, passive transport or diffusion. The protein domain of these fusion proteins can be an S-protein, an S-tag, an antigen, a hapten or a single chain variable region (scFv), of an antibody.

The invention also includes isolated fusion proteins comprising a first domain encoding a single chain variable region of an antibody; a second domain encoding a nuclear localization signal; and a third domain encoding transcriptional regulatory activity.

IV. Modular Assembly Method for Synthesis of Multi-finger ZFPs

A further aspect of the invention relates to providing a rapid, modular method for assembling large numbers of multi-fingered ZFPs from three sets of oligonucleotides encoding the desired individual zinc finger domains. This method thus provides a high through-put method to produce a DNA encoding a multi-fingered ZFP. In fact, with the use of robotics, the method of the invention can be automated to run parallel assembly of these DNA molecules.

As shown, in Table 3, there are 256 different four base pair targets. If a recognition code, such as the preferred version of Table 1, is used in which a single amino acid can be specified for each four variable domain positions for each of the four nucleotides, then a single unique zinc finger domain can be constructed for each of the 256 target sequences. Now if these domains are used to create three-finger ZFPs, the number of possible ZFPs can be calculated as 256³ or 1.68×10⁷. The present method provides a way of synthesizing all of these ZFPs from 768 oligonucleotides, i.e., three sets of 256 oligonucleotides. In fact, the present method can be adapted such that for each new set of 256 oligonucleotides, every possible ZFP can be made for ZFPs with one more finger.

Hence, for making a nucleic acid encoding a zinc finger protein (ZFP) having three zinc fingers domains, each domain independently represented by the formula

-X₃-Cys-X₂₋₄-Cys-X₁₂-His-X₃₋₅-His-X₄-,

and said domains, independently, covalently joined with from 0 to 10 amino acid residues the method comprises:

(a) preparing a mixture, under conditions for performing a polymerase-chain reaction (PCR), comprising:

(i) a first double-stranded oligonucleotide encoding a first zinc finger domain,

(ii) a second double-stranded oligonucleotide encoding a second zinc finger domain,

(iii) a third double-stranded oligonucleotide encoding a third zinc finger,

(iv) a first PCR primer complementary to the 5′ end of the first oligonucleotide,

(v) a second PCR primer complementary to the 3′ end of the third oligonucleotide,

wherein the 3′ end of the first oligonucleotide is sufficiently complementary to the 5′ end of the second oligonucleotide to prime synthesis of said second oligonucleotide therefrom, wherein the 3′ end of the second oligonucleotide is sufficiently complementary to the 5′ end of the third oligonucleotide to prime synthesis of said third oligonucleotide therefrom, and wherein the 3′ end of the first oligonucleotide is not complementary to the 5′ end of the third oligonucleotide and the 3′ end of the second oligonucleotide is not complementary to the 5′ end of the first oligonucleotide;

(b) subjecting the mixture to a PCR; and

(c) recovering the nucleic acid encoding the ZFP.

The PCR the reaction is conducted under standard or typical PCR conditions for multiple cycles of heating, annealing and synthesis. The PCR amplification primers preferably include a restriction endonuclease recognition site. Such sites can facilitate cloning or, as described below, assembly of ZFPs with four or more zinc finger domains. Useful restriction enzymes include BbsI, BsaI, BsmBI, or BspMI, and most preferably BsaI.

To synthesize a nucleic acid encoding a zinc finger protein (ZFP) having four or more zinc fingers domains, each domain independently represented by the formula

-X₃-Cys-X₂₋₄-Cys-X₁₂-His-X₃₋₅-His-X₄-,

and said domains, independently, covalently joined with from 0 to 10 amino acid residues, the method comprises:

(a) preparing a first nucleic acid according to the above method, wherein said second PCR primer includes a first restriction endonuclease recognition site;

(b) preparing a second nucleic acid according to the above method, wherein said first and second PCR primers (in this second synthesis) are complementary to the 5′ and 3′ ends, respectively, of the number of zinc finger domains selected for amplification, wherein said first PCR primer includes a restriction endonuclease recognition site that, when subjected to cleavage by its corresponding restriction endonuclease, produces an end having a sequence which is complementary to and can anneal to, the end produced when said second PCR primer of step (a) is subjected to cleavage by its corresponding restriction endonuclease and wherein said second PCR primer of step (b), optionally, includes a second restriction enzyme recognition site that, when subjected to cleavage produces an end that differs from and is not complementary to that produced from the first restriction endonuclease recognition site;

(c) optionally, preparing one or more additional nucleic acids by the above method, wherein said first and second PCR primers (of this additional synthesis) are complementary to the 5′ and 3′ ends, respectively, of the number of zinc finger domains selected for amplification, wherein said first PCR primer for each additional nucleic acid includes a restriction endonuclease recognition site that, when subjected to cleavage by its corresponding restriction endonuclease, produces an end having a sequence which is complementary to and can anneal to the end produced when the second PCR primer used for preparation of the second nucleic acid, or for the additional nucleic acid that is immediately upstream of the additional nucleic acid, is subjected to cleavage by its corresponding restriction endonuclease, and wherein said second PCR primer for each additional nucleic acid, optionally, includes a restriction endonuclease recognition site that, when subjected to cleavage produces an end that differs from and is not complementary to any previously used;

(d) cleaving said first nucleic acid, said second nucleic acid and said additional nucleic acids, if prepared, with their corresponding restriction endonucleases to produce cleaved first, second and additional, if prepared, nucleic acids; and

(e) ligating said cleaved first, second and additional, if prepared, nucleic acids to produce the nucleic acid encoding a zinc finger protein (ZFP) having four or more zinc fingers domains. Useful and preferred restriction enzymes are as provided above, provide each one selected produces a unique pair of cleavable, anneable ends.

If step (c) is omitted, then a ZFP with four, five or six zinc finger domains can be made. If nucleic acid encoding a 3-finger ZFP is produced in step (b) and one additional nucleic acid is prepared by step (c), then a ZFP with seven, , eight or nine zinc finger domains can be made.

By appropriate design, the oligonucleotides can provide for optimal codon usage for an organism. such as a bacterium, a fungus, a yeast, an animal, an insect or a plant. In a preferred embodiment optimal codon usage (to maximize expression in the organism) is provided for E. coli, humans or mice, cereal plants, rice, tomato or corn. The method works with transgenic plants.

The nucleic acids made by this method can be incorporated in expression vectors and host cells. Those vectors and hosts can in turn be used to recombinantly express the ZFP by methods well known in the art.

The invention includes, sets of oligonucleotides comprising a number of separate oligonucleotides designed to use any combination of amino acids from the recognition code for four base pair targets in which

(a) if the first base is G, then Z⁶ is arginine or lysine, if the first base is A, then Z⁶ is glutamine or asparagine, if the first base is T, then Z⁶ is threonine, tyrosine, leucine, isoleucine or methionine, if the first base is C, then Z⁶ is glutamic acid or aspartic acid,

(b) if the second base is G, then Z³ is histidine or lysine, if the second base is A, then Z³ is asparagine or glutamine, if the second base is T, then Z³ is serine, alanine or valine, if the second base is C, then Z³ is aspartic acid or glutamic acid,

(c) if the third base is G, then Z⁻¹ is arginine or lysine, if the third base is A, then Z⁻¹ is glutamine or asparagine, if the third base is T, then Z⁻¹ is threonine, methionine leucine or isoleucine, if the third base is C, then Z⁻¹ is glutamic acid or aspartic acid,

(iv) if the complement of the fourth base is G, then Z² is serine or arginine, if the complement of the fourth base is A, then Z² is asparagine or glutamine, if the complement of the fourth base is T, then Z² is threonine, valine or alanine, and if the complement of the fourth base is C, then Z² is aspartic acid or glutamic acid.

Preferably, the number of oligonucleotides is 256 since this represents the number of 4 base pair targets. Sets designed for the preferred recognition code of Table 1 are preferred.

V. Miscellaneous

“Organisms” as used herein include bacteria, fungi, yeast, animals, birds, insects, plants and the like. Animals include, but are not limited to, mammals (humans, primates, etc.), commercial or farm animals (fish, chickens, cows, cattle, pigs, sheep, goats, turkeys, etc.), research animals (mice, rats, rabbits, etc.) and pets (dogs, cats, parakeets and other pet birds, fish, etc.). As contemplated herein, particular animals may be members of multiple animal groups. Plants are described in more detail herein.

In some instances it may be that the cells of the organisms are used in a method of the invention. When cells are contemplated as an aspect of an invention herein, then in addition cells from any of the animals, organisms or plants expressly provided herein, the cells include cells isolated from such organisms and animals as well as cell lines used in research or other laboratories, including primary and secondary cell lines and the like.

Cell transformation techniques and gene delivery methods (such as those for in vivo use to deliver genes) are well known in the art. Any such technique can be used to deliver a nucleic acid encoding a ZFP or ZFP-fusion protein of the invention to a cell or subject, respectively.

The term “expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The zinc finger-effector fusions of the present invention are chimeric. The expression cassette may also be one which is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter which initiates transcription only when the host cell is exposed to some particular external stimulus. In the case of a multicellular organism, such as a plant, the promoter can also be specific to a particular tissue or organ or stage of development. In the case of a plastid expression cassette, for expression of the nucleotide sequence from a plastid genome, additional elements, i.e. ribosome binding sites, maybe required.

By “heterologous” DNA molecule or sequence is meant a DNA molecule or sequence not naturally associated with a host cell into which it is introduced, including non-naturally occurring multiple copies of a naturally-occurring DNA sequence.

By “homologous” DNA molecule or sequence is meant a DNA molecule or sequence naturally associated with a host cell.

By “minimal promoter” is meant a promoter element, particularly a TATA element, that is inactive or that has greatly reduced promoter activity in the absence of upstream activation. In the presence of a suitable transcription factor, the minimal promoter functions to permit transcription.

A “plant” refers to any plant or part of a plant at any stage of development, including seeds, suspension cultures, embryos, meristematic regions, callus tissue, leaves, roots, shoots, gametophytes, sporophytes, pollen, and microspores, and progeny thereof. Also included are cuttings, and cell or tissue cultures. As used in conjunction with the present invention, the term “plant tissue” includes, but is not limited to, whole plants, plant cells, plant organs (e.g., leafs, stems, roots, meristems) plant seeds, protoplasts, callus, cell cultures, and any groups of plant cells organized into structural and/or functional units.

The present invention can be used, for example, to modulate gene expression, alter genome structure and the like, over a broad range of plant types, preferably the class of higher plants amenable to transformation techniques, particularly monocots and dicots. Particularly preferred are monocots such as the species of the Family Gramineae including Sorghum bicolor and Zea mays. The isolated nucleic acid and proteins of the present invention can also be used in species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, and Triticum.

Preferred plant cell includes those from corn (Zea mays), canola (Brassica napus, Brassica rapa ssp.), alfalfa (Medicago sativa), rice (Oryza sativa). rye (Secale cereale), sorghum (Sorghum bicolor, Sorghum vulgare), sunflower (Helianthus annuus), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato Qpomoea batatus), cassava (Manihot esculenta), coffee (Cqfea spp.), coconut (Cocos nucijra), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integr˜fblia), almond (Prunus amygdalus), sugar beets (Beta vulgaris), sugarcane (Saccharum spp.), duckweed (Lemna spp.), oats, barley, vegetables, ornamentals, and conifers.

Preferred vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C cantalupensis), and musk melon (C. melo).

Preferred ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.). petunias (Petunia hybrida), carnation (Dianthus caryophyllus), poinsettia (Euphorbiapulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Isuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Most preferably, plants of the present invention are crop plants (for example, corn, alfalfa, sunflower, canola, soybean, cotton, peanut, sorghum, wheat, tobacco, etc.), even more preferably corn and soybean plants, yet more preferably corn plants.

As used herein, “transgenic plant” or “genetically modified plant” includes reference to a plant which comprises within its genome a heterologous polynucleotide. Generally, and preferably, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, a “target polynucleotide,” “target nucleic acid,” “target site” or other similar terminology refers to a portion of a double-stranded polynucleotide, including DNA, RNA, peptide nucleic acids (PNA) and combinations thereof, to which a zinc finger domain binds. In one preferred embodiment, the target polynucleotide is all or part of a transcriptional control element for a gene and the zinc finger domain is capable of binding to and modulating (activating or repressing) its degree of expression. A transcriptional control element may include one or more of the following: positive and negative control elements such as a promoter, an enhancer, other response elements (e.g., steroid response element, heat shock response element or metal response element), repressor binding sites, operators and silencers. The transcriptional control element can be viral, eukaryotic, or prokaryotic. A “target nucleotide sequence” also refers to a downstream sequence which can bind a protein and thereby modulate expression, typically prevent or activate transcription.

VI. Uses

The discovery of the zinc finger-nucleotide base recognition code of the invention allows the design of ZFPs and ZFP-fusion proteins capable of binding to and modulating the expression of any target nucleotide sequence. The target nucleotide sequence is at any location within the target gene whose expression is to be regulated which provides a suitable location for controlling expression. The target nucleotide sequence may be within the coding region or upstream or downstream thereof, but it can also be some distance away. For example enhancers are known to work at extremely long distances from the genes whose expression they modulate. For activation, targets upstream from ATG translation start codon are preferred, most preferably upstream of TATA box within about 100 bp from the start of transcription. For repression, upstream from the ATG translation start codon is also preferred, but preferably downstream from TATA box.

A protein comprising one or more zinc finger domains which binds to transcription control elements in the promoter region may cause a decrease in gene expression by blocking the binding of transcription factors that normally stimulate gene expression. In other instances, it may be desirable to increase expression of a particular protein. A ZFP which contains a transcription activator is used to cause such an increase in expression. In addition, gene expression can be modulated by fusing the ZFP to a transcriptional protein recruiting protein, or an active domain thereof. Such proteins act by recruiting transcriptional activators or repressors to the site where the transcriptional recruiting protein is located to thereby allow the activators and repressors to modulate gene expression.

In another embodiment of the invention, ZFPs are fused with enzymes to target the enzymes to specific sites in the genome. These fusion proteins direct the enzyme to specific sites and allow modification of the genome and of chromatin. Such modifications can be anywhere on the genome, e.g., in a gene or far from genes. For example, genomes can be specifically manipulated by fusing designed zinc finger domains based on the recognition code of the invention using standard molecular biology techniques with integrases or transposases to promote integration of exogenous genes into specific genomic sites (transposases or integrases), to eliminate (knock-out) specific endogenous genes (transposases) or to manipulate promoter activities by inserting one or more of the following DNA fragments: strong promoters/enhancers, tissue-specific promoters/enhancers, insulators or silencers. In other instances, a ZFP which binds to a polynucleotide having a particular sequence. In other embodiments, enzymes such as DNA methyltransferases, DNA demethylases, histone acetylases and histone deacetylases are attached to the ZFPs prepared based on the recognition code of the present invention for manipulation of chromatin structure.

For example, DNA methylation/demethylation at specific genomic sites allows manipulation of epi-genetic states (gene silencing) by altering methylation patterns, and histone acetylation/deacetylation at specific genomic sites allows manipulation of gene expression by altering the mobility and/or distribution of nucleosomes on chromatin and thereby increase access of transcription factors to the DNA. Proteases can similarly affect nucleosome mobility and distribution on DNA to modulate gene expression.

Nucleases can alter genome structure by nicking or digesting target sites and may allow introduction of exogenous genes at those sites. Invertases can alter genome structure by swapping the orientation of a DNA fragment. Resolvases can alter the genomic structure by changing the linking state of the DNA, e.g., by releasing concatemers.

Examples of some of the above regulatory proteins include, but are not limited to: transposase: Tc1 transposase, Mos1 transposase, Tn5 transposase, Mu transposase; integrase: HIV integrase, lambda integrase; recombinase: Cre recombinase, Flp recombinase, Hin recombinase; DNA methyltransferase: SssI methylase, AluI methylase, HaeIII methylase, HhaI methylase, HpaII methylase, human Dnmt1 methyltransferase; DNA demethylase: MBD2B, a candidate demethylase; histone acetylase: human GCN5, CBP (CREB-binding protein); histone deacetylase: HDAC1; nuclease: micrococcal nuclease, staphylococcal nuclease, DNase I, T7 endonulcease; resolvase: Ruv C resolvase, Holiday junction resolvase Hjc; and invertase: Hin invertase.

In another embodiment, a nuclear localization peptide is attached to the ZFP or ZFP-fusion ZFP to target the zinc finger to the nuclear compartment. In addition the ZFPs can have a cellular uptake signal attached, either alone or in conjunction with other moieties such as the above described regulatory domains and the like. Such cellular uptake signals include, but are not limited to, the minimal Tat protein transduction domain which is residues 47-57 of the human immunodeficiency virus Tat protein: YGRKKRRQRRR (SEQ ID NO: 18).

A wild type transposase 2 homodimer (FIG. 4, left panel) comprises a catalytic (cleavage) domain 4, dimerization domains 6 and terminal inverted repeat (TIR) binding domains 8. In one embodiment of the invention, zinc finger domains are substituted for the TIR domains to promote cleavage of a genomic site targeted by the zinc finger domains according to the recognition code of the invention. An artificial transposase heterodimer 10 (FIG. 4, right panel) is generated by joining catalytic domains 4 to zinc finger domains 12 via linkers 14 which comprise heterodimeric peptides including, but not limited to, jun-fos and acidic-basic heterodimer peptides. For example, the acidic peptide AQLEKELQALEKENAQLEWELQALEKELAQ (SEQ ID NO: 19) and basic peptide AQLKKKLQALKKKNAQLKWKLQALKKKLAQ (SEQ ID NO: 20) can be used as linkers and will heterodimerize. These heterodimers pull the DNA ends together after cleavage of the DNA by the catalytic domains. The zinc finger domains 12 may target the same or different sites in the genome according to the recognition code of the invention. Any desired genomic site may be targeted using these artificial transposases. The cellular system will repair (ligate) the cut ends of the DNA if they are brought in close proximity by the artificial transposase.

In another embodiment of the engineered transposases described above, the specificities of the TIRs may be altered, combined with usage of the heterodimers, to produce site-specific knock-out (KO) of a gene of interest. Alternatively, replacing the TIRs with zinc finger domains, particularly ones with different specificity (as described in the preceding paragraph) produces another class of proteins useful to make site-specific KOs.

In addition, by fusion with ZFPs, transposases (that have a catalytic domain, a dimerization domain and a TIR binding domain) can be recruited to specific genomic sites in combination with usage of the heterodimers to produce transposases having altered DNA binding specificity, resulting in site-specific knock-in (KI) of a gene of interest. For example, a zinc finger domain can be joined to the C. elegans transposon Tc1 via a flexible linker (e.g. (GGGGS)₄ (SEQ ID NO: 21) in which G=glycine and S=serine), either as zinc finger-linker-Te1, or as Tc1-linker-zinc finger. It will be appreciated that any transposase, zinc finger domain or linker peptide may be used in these constructs.

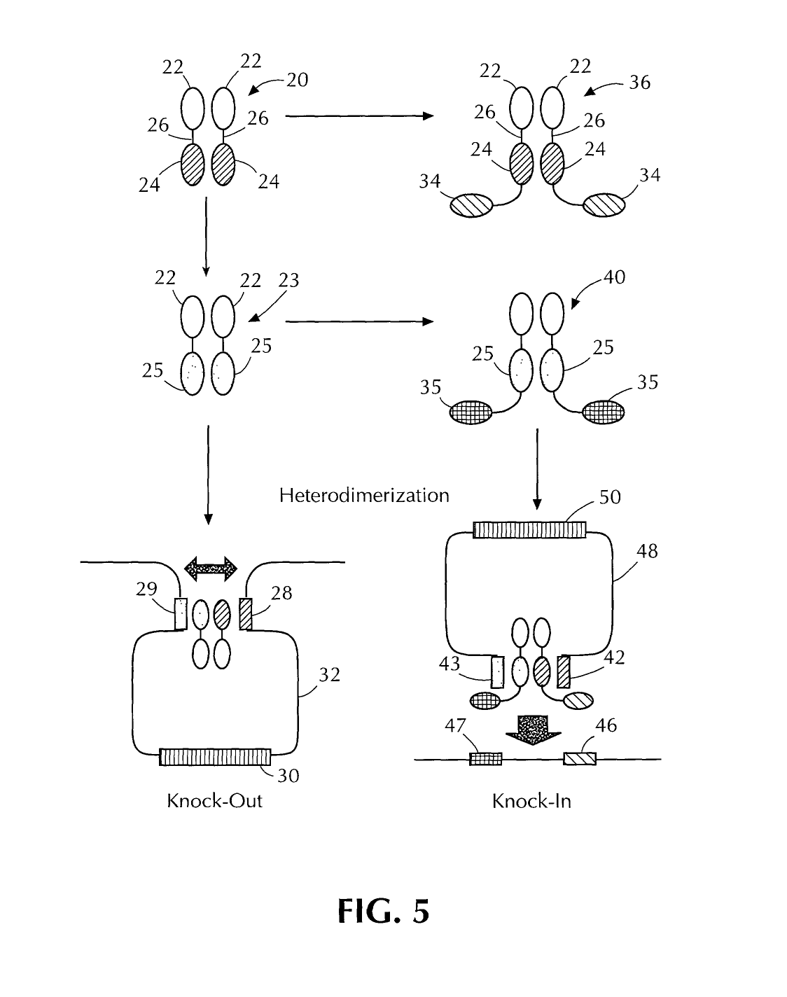

The site-specific KO and KI strategies are summarized in FIG. 5. Transposase 20 comprises catalytic domains 22 and TIR binding domains 24 joined by homodimeric or heterodimeric protein domain linkers 26. TIR binding domains 24 are engineered by standard techniques to have altered target specificities which may be the same or different, resulting in transposase 23 having altered TIR bonding domains 25. These TIRs target genomic sequences 28 and 29 which flank a gene 30 to be deleted. After binding of the TIRs to their complementary genomic sequences 28 and 29, a DNA loop 32 comprising gene 30 is formed, and the catalytic domains 22 cleave the DNA loop 32, resulting in KO of gene 30. Preferably, the catalytic domains only have cleavage, not re-ligation activity. Ligation is preferably performed by the cell to join the cleaved ends of the DNA.

In another embodiment of the invention, engineered transposases are used to perform site-specific KI of an exogenous gene. In this embodiment, transposase 20 is linked to zinc finger domains 34 which may have the same or different specificities to produce zinc finger fusion 36. In another embodiment, transposase 23 is fused to zinc finger domains 35 which may have the same or different specificities to produce transposase 40 which comprises TIRs 24 and 25 having altered DNA sequence specificity. TIRs 24 and 25 contact genoric regions 42 and 43, respectively, and zinc finger domains bind to target sequences 46 and 47, followed by cleavage of looped DNA 48 and incorporation of gene 50 between zinc finger target sequences 46 and 47. For the KI embodiment, it is preferred that the catalytic domains of the transposase have both cleavage and ligation activities.

The ZFPs and recognition code of the present invention can be used to modulate gene expression in any organism, particularly plants. The application of ZFPs and constructs to plants is particularly preferred. Where a gene contains a suitable target nucleotide sequence in a region which is appropriate for controlling expression, the regulatory factors employed in the methods of the invention can target the endogenous nucleotide sequence. However, if the target gene lacks an appropriate unique nucleotide sequence or contains such a sequence only in a position where binding to a regulatory factor would be ineffective in controlling expression, it may be necessary to provide a “heterologous” targeted nucleotide sequence. By “heterologous” targeted nucleotide sequence is meant either a sequence completely foreign to the gene to be targeted or a sequence which resides in the gene itself, but in a different position from that wherein it is inserted as a target. Thus, it is possible completely to control the nature and position of the targeted nucleotide sequence.

In one embodiment, the zinc finger polypeptides of the present invention is used to inhibit the expression of a disease-associated gene. Preferably, the zinc finger polypeptide is not a naturally-occurring protein, but is specifically designed to inhibit the expression of the gene. The zinc finger polypeptide is designed using the amino acid-base contacts shown in Table 1 to bind to a regulatory region of a disease-associated gene and thus prevent transcription factors from binding to these sites and stimulating transcription of the gene. In one example, the disease-associated gene is an oncogene such as a BCR-ABL fusion oncogene or a ras oncogene, and the zinc finger polypeptide is designed to bind to the DNA sequence GCAGAAGCC (SEQ ID NO: 22) and is capable of inhibiting the expression of the BCR-ABL fusion oncogene.

A nucleic acid sequence of interest may also be modified using the zinc finger polypeptides of the invention by binding the zinc finger to a polynucleotide comprising a target sequence to which the zinc finger binds. Binding of a zinc finger to a target polynucleotide may be detected in various ways, including gel shift assays and the use of radiolabeled, fluorescent or enzymatically labeled zinc fingers which can be detected after binding to the target sequence. The zinc finger polypeptides can also be used as a diagnostic reagent to detect mutations in gene sequences, to purify restriction fragments from a solution, or to visualize DNA fragments of a gel.

As used herein, “effector” or “effector protein” refer to constructs or their encoded products which are able to regulate gene expression either by activation or repression or which exert other effects on a target nucleic acid. The effector protein may include a zinc finger binding region only, but more commonly also includes a “functional domain” such as a “regulatory domain.” The regulatory domain is the portion of the effector protein or effector which enhances or represses gene expression (and is also referred to as a transcriptional regulatory domain), or may be a nuclease, recombinase, integrase or any other protein or enzyme which has a biological effect on the polynucleotide to which the ZFP binds.

The effector domain has an activity such as transcriptional regulation or modulation activity, DNA modifying activity, protein modifying activity and the like when tethered (e.g., fused) to a DNA binding domain, i.e., a ZFP. Examples of regulatory domains include proteins or effector domains of proteins, e.g., transcription factors and co-factors (e.g., KRAB, MAD, ERD, SID, nuclear factor kappa B subunit p65, early growth response factor 1, and nuclear hormone receptors, VP16, VP64), endonucleases, integrases, recombinases, methylases, methyltransferases, histone acetyltransferases, histone deacetylases and the like.

Activators and repressors include co-activators and co-repressors (Utley et al., Nature 394:498-502 (1998); WO 00/03026). Effector domains can include, but are not limited to, DNA-binding domains from a protein that is not a ZFP, such as a restriction enzyme, a nuclear hormone receptor, a homeodomain protein such as engrailed or antenopedia, a bacterial helix-turn-helix motif protein such as lambda repressor and tet repressor, Gal4, TATA binding protein, helix-loop-helix motif proteins such as myc and myo D, leucine zipper type proteins such as fos and jun, and beta sheet motif proteins such as met, arc, and mnt repressors. Particularly preferred is the C1 activator domain of maize.

Likewise an effector domain can include, but is not limited to a transposase, integrase, recombinase, resolvase, invertase, protease, DNA methyltransferase, DNA demethylase, histone acetylase, histone deacetylase, nuclease, transcriptional repressor, transcriptional activator, a single-stranded DNA binding protein, a nuclear-localization signal, a transcription-protein recruiting protein or a cellular uptake domain. Effector domains further include protein domains which exhibits transposase activity, integrase activity, recombinase activity, resolvase activity, invertase activity, protease activity, DNA methyltransferase activity, DNA demethylase activity, histone acetylase activity, histone deacetylase activity, nuclease activity, nuclear localization activity, transcriptional protein recruiting activity, transcriptional repressor activity or transcriptional activator activity.

In a preferred embodiment the ZFP having an effector domain is one that is responsive to a ligand. The effector domain can effect such a response. Example of such ligand-responsive domains are hormone receptor ligand binding domains, including, for example, the estrogen receptor domain, the ecydysone receptor system, the glucocorticosteroid receptor, and the like. Preferred inducers are small, inorganic, biodegradable, molecules. Use of ligand inducible ZFP-effector fusions is generally known as a gene switch.

The ZFP can be covalently or non-covalently associated with one or more regulatory domains, alternatively two or more regulatory domains, with the two or more domains being two copies of the same domain, or two different domains. The regulatory domains can be covalently linked to the ZFP nucleic acid binding domain, e.g., via an amino acid linker, as part of a fusion protein. The ZFPs can also be associated with a regulatory domain via a non-covalent dimerization domain, e.g., a leucine zipper, a STAT protein N terminal domain, or an FK506 binding protein (see, e.g., O'Shea, Science 254: 539 (1991), Barahmand-Pour et al., Curr. Top. Microbiol. Immunol. 211:121-128 (1996); Klemm et al., Annu. Rev. Immunol. 16:569-592 (1998); Klemm et al., Annu. Rev. Immunol. 16:569-592 (1998); Ho et al., Nature 382:822-826 (1996); and Pomeranz et al., Biochem. 37:965 (1998)). The regulatory domain can be associated with the ZFP domain at any suitable position, including the C- or N-terminus of the ZFP.

Common regulatory domains for addition to the ZFP made using the methods of the invention include, e.g., DNA-binding domains from transcription factors, effector domains from transcription factors (activators, repressors, co-activators, co-repressors), silencers, nuclear hormone receptors, and chromatin associated proteins and their modifiers (e.g., methylases, kinases, acetylases and deacetylases).

Transcription factor polypeptides from which one can obtain a regulatory domain include those that are involved in regulated and basal transcription. Such polypeptides include transcription factors, their effector domains, coactivators, silencers, nuclear hormone receptors (see, e.g., Goodrich et al., Cell 84:825-30 (1996) for a review of proteins and nucleic acid elements involved in transcription; transcription factors in general are reviewed in Bames and Adcock, Clin. Exp. Allergy 25 Suppl. 2:46-9 (1995) and Roeder, Methods Enzymol. 273:165-71 (1996)). Databases dedicated to transcription factors are also known (see, e.g., Science 269:630 (1995)). Nuclear hormone receptor transcription factors are described in, for example, Rosen et al., J. Med. Chem. 38:4855-74 (1995). The C/EBP family of transcription factors are reviewed in Wedel et al., Immunobiology 193:171-85 (1995). Coactivators and co-repressors that mediate transcription regulation by nuclear hormone receptors are reviewed in, for example, Meier, Eur. J. Endocrinol. 134(2):158-9 (1996); Kaiser et al., Trends Biochem. Sci. 21:342-5 (1996); and Utley et al., Nature 394:498-502 (1998)). GATA transcription factors, which are involved in regulation of hematopoiesis, are described in, for example, Simon, Nat. Genet. 11:9-11 (1995); Weiss et al., Exp. Hematol. 23:99-107. TATA box binding protein (T13P) and its associated TAF polypeptides (which include TAF30, TAF55, TAF80, TAF1 10, TAFI 50, and TAF250) are described in Goodrich & Tjian, Curr. Opin. Cell Biol. 6:403-9 (1994) and Hurley, Curr. Opin. Struct. Biol. 6:69-75 (1996). The STAT family of transcription factors are reviewed in, for example, Barahmand-Pour et al., Curr. Top. Microbiol. Immunol. 211:121-8 (1996). Transcription factors involved in disease are reviewed in Aso et al., J Clin. Invest. 97:1561-9 (1996).

In one embodiment, the KRAB repression domain from the human KOX-I protein is used as a transcriptional repressor (Thiesen et al., New Biologist 2:363-374 (1990); Margolin et al., Proc. Natl. Acad. Sci. U.S.A. 91:4509-4513 (1994); Pengue et al., Nucl. Acids Res. 22:2908-2914 (1994); Witzgall et al., Proc. Natl. Acad. Sci. U.S.A. 91:4514-4518 (1994)). In another embodiment, KAP-1, a KRAB co-repressor, is used with KRAB (Friedman et al., Genes Dev. 10:2067-2078 (1996)). Alternatively, KAP-I can be used alone with a ZFP. Other preferred transcription factors and transcription factor domains that act as transcriptional repressors include MAD (see, e.g., Sommer et al., J Biol. Chem. 273:6632-6642 (1998); Gupta et al., Oncogene 16:1149-1159 (1998); Queva et al., Oncogene 16:967-977 (1998); Larsson et al., Oncogene :737-748 (1997); Laherty et al., Cell 89:349-356 (1997); and Cultraro et al., Mol Cell. Biol. 17:2353-2359 (19977)); FKHR (forkhead in rhapdosarcoma gene; Ginsberg et al., Cancer Res. 15:3542-3546 (1998); Epstein et al., Mol. Cell. Biol. 18:4118-4130 (1998)); EGR-I (early growth response gene product-1; Yan et al., Proc. Natl. Acad. Sci. U.S.A. 95:8298-8303 (1998); and Liu et al., Cancer Gene Ther. 5:3-28 (1998)); the ets2 repressor factor repressor domain (ERD; Sgouras et al., EM80 J 14:4781-4793 ((19095)); and the MAD smSIN3 interaction domain (SID; Ayer et al., Mol. Cell. Biol. 16:5772-5781 (1996)).

In one embodiment, the HSV VP 16 activation domain is used as a transcriptional activator (see, e.g., Hagmann et al., J Virol. 71:5952-5962 (1997)). Other preferred transcription factors that could supply activation domains include the VP64 activation domain (Selpel et al., EMBO J 11:4961-4968 (1996)); nuclear hormone receptors (see, e.g., Torchia et al., Curr. Opin. Cell. Biol. 10:373-383 (1998)); the p65 subunit of nuclear factor kappa B (Bitko & Barik, J Virol. 72:5610-5618 (1998) and Doyle & Hunt, Neuroreport 8:2937-2942 (1997)); and EGR-I (early growth response gene product-1; Yan et al., Proc. Nad. Acad. Sci. U.S.A. 95:8298-8303 (1998); and Liu et al., Cancer Gene Ther. 5:3-28 (1998)).

Kinases, phosphatases, and other proteins that modify polypeptides involved in gene regulation are also useful as regulatory domains for ZFPs. Such modifiers are often involved in switching on or off transcription mediated by, for example, hormones. Kinases involved in transcription regulation are reviewed in Davis, Mol. Reprod. Dev. 42:459-67 (1995), Jackson et al., Adv. Second Messenger Phosphoprotein Res. 28:279-86 (1993), and Boulikas, Crit. Rev. Eukaryot. Gene Expr. 5:1-77 (1995), while phosphatases are reviewed in, for example, Schonthal & Semin, Cancer Biol. 6:239-48 (1995). Nuclear tyrosine kinases are described in Wang, Trends Biochem. Sci. 19:373-6 (1994).

As described, useful domains can also be obtained from the gene products of oncogenes (e.g., myc, jun, fos, myb, max, mad, rel, ets, bcl, myb, mos family members) and their associated factors and modifiers. Oncogenes are described in, for example, Cooper, Oncogenes, 2nd ed., The Jones and Bartlett Series in Biology, Boston, Mass., Jones and Bartlett Publishers, 1995. The ets transcription factors are reviewed in Waslylk et al., Eur. J Biochem. 211:7-18 (1993). Myc oncogenes are reviewed in, for example, Ryan et al., Biochem. J. 314:713-21 (1996). The Jun and fos transcription factors are described in, for example, The Fos and Jun Families of Transcription Factors, Angel & Herrlich, eds. (1994). The max oncogene is reviewed in Hurlin et al., Cold Spring Harb. Symp. Quant. Biol. 59:109-16. The myb gene family is reviewed in Kanei-Ishii et al., Curr. Top. Microbiol. Immunol. 211:89-98 (1996). The mos family is reviewed in Yew et al., Curr. Opin. Genet. Dev. 3:19-25 (1993).

In another embodiment, histone acetyltransferase is used as a transcriptional activator (see, e.g., Jin & Scotto, Mol. Cell. Biol. 18:4377-4384 (1998); Wolffle, Science 272:371-372 (1996); Taunton et al., Science 272:408-411 (1996); and Hassig et al., Proc. Natl. Acad. Sci. U.S.A. 95:3519-3524 (1998)). In another embodiment, histone deacetylase is used as a transcriptional repressor (see, e.g., Jin & Scotto, Mol. Cell. Biol. 18:4377-4384 (1998); Syntichaki & Thireos, J Biol. Chem. 273:24414-24419 (1998); Sakaguchi et al., Genes Dev. 12:2831-2841 (1998); and Martinez et al., J Biol. Chem. 273:23781-23785 (1998)).

In addition to regulatory domains, often the ZFP is expressed as a fusion protein such as maltose binding protein (“MBP”), glutathione S transferase (GST), hexahistidine, c-myc, and the FLAG epitope, for ease of purification, monitoring expression, or monitoring cellular and subcellular localization.

The nucleic acid sequence encoding a ZFP can be modified to improve expression of the ZFP in plants by using codon preference. When the nucleic acid is prepared or altered synthetically, advantage can be taken of known codon preferences of the intended plant host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledons or dicotyledons as these preferences have been shown to differ (Murray et al. Nucl. Acids Res. 17: 477-498 (1989)). Thus, the maize preferred codon for a particular amino acid may be derived from known gene sequences from maize. Maize codon usage for 28 genes from maize plants are listed in Table 4 of Murray et al., supra.

The targeted sequence may be any given sequence of interest for which a complementary ZFP is designed. Targeted genes include both structural and regulatory genes, such that targeted control or effector activity either directly or indirectly via a regulatory control. Thus single genes or gene families can be controlled.

The targeted gene may, as is the case for the maize MIPS gene and AP3 gene, be endogenous to the plant cells or plant wherein expression is regulated or may be a transgene which has been inserted into the cells or plants in order to provide a production system for a desired protein or which has been added to the genetic compliment in order to modulate the metabolism of the plant or plant cells.

It may be desirable in some instances to modify plant cells or plants with families of transgenes representing, for example, a metabolic pathway. In those instances, it may be desirable to design the constructs so that the family can be regulated as a whole—e.g., by designing the control regions of the members of the family with similar or identical targets for the ZFP portion of the effector protein. Such sharing of target sequences in gene families may occur naturally in endogenously produced metabolic sequences.

In most instances, it is desirable to provide the expression system for the effector protein with control sequences that are tissue specific so that the desired gene regulation can occur selectively in the desired portion of the plant. For example, to repress MIPS expression, it is desirable to provide the effector protein with control sequences that are selectively effective in seeds. With respect to the AP3 gene, effector proteins for regulation of expression would be designed for selective expression in flowering portions of the plant. However, in some instances, it may be desirable to have the genetic control expressible in all tissues for example in instances where an insect resistance gene is the target. In such cases, as well, it may be desirable to place the expression system for the effector protein under control of an inducible promoter so that inducer can be supplied to the plant only when the need arises, for example, activation of an insect resistance gene.

In one embodiment, ZFPs can be used to create functional “gene knockouts” and “gain of function” mutations in a host cell or plant by repression or activation of the target gene expression. Repression or activation may be of a structural gene, one encoding a protein having for example enzymatic activity, or of a regulatory gene, one encoding a protein that in turn regulates expression of a structural gene. Expression of a negative regulatory protein can cause a functional gene knockout of one or more genes, under its control. Conversely, a zinc finger having a negative regulatory domain can repress a positive regulatory protein to knockout or prevent expression of one or more genes under control of the positive regulatory protein.

The ZFPs of the invention and fusion proteins of the invention, particularly those useful for modulating gene expression can be used for functional genomics applications and target validation applications such as those described in WO 01/19981 to Case et al.

The present invention also provides recombinant expression cassettes comprising a ZFP-encoding nucleic acid of the present invention. A nucleic acid sequence coding for the desired polynucleotide of the present invention can be used to construct a recombinant expression cassette which can be introduced into a desired host cell. A recombinant expression cassette will typically comprise a polynucleotide of the present invention operably linked to transcriptional initiation regulatory sequences which will direct the transcription of the polynucleotide in the intended host cell, such as tissues of a transformed plant.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

A plant promoter fragment can be employed which will direct expression of a polynucleotide of the present invention in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the P- or 2′-promoter derived from T-DNA of Agrobacterium tumefaciens, the ubiquitin I promoter, the Smas promoter, the cinnamyl alcohol dehydrogenase promoter (U.S. Pat. No. 5,683,439), the Nos promoter, the pEmu promoter, the rubisco promoter, the GRP 1-8 promoter, and other transcription initiation regions from various plant genes known to those of skill in the art.

Alternatively, the plant promoter can direct expression of a polynucleotide of the present invention in a specific tissue or may be otherwise under more precise environmental or developmental control. Such promoters are referred to here as “inducible” promoters. Environmental conditions that may effect transcription by inducible promoters include pathogen attack, anaerobic conditions, or the presence of light. Examples of inducible promoters include the AdhI promoter which is inducible by hypoxia or cold stress, the Hsp70 promoter which is inducible by heat stress, and the PPDK promoter which is inducible by light. Examples of promoters under developmental control include promoters that initiate transcription only, or preferentially, in certain tissues, such as leaves, roots, fruit, seeds, or flowers. An exemplary promoter is the anther specific promoter 5126 (U.S. Pat. Nos. 5,689,049 and 5,689,051). The operation of a promoter may also vary depending on its location in the genome. Thus, an inducible promoter may become fully or partially constitutive in certain locations.

Both heterologous and non-heterologous (i.e., endogenous) promoters can be employed to direct expression of the nucleic acids of the present invention. These promoters can also be used, for example, in recombinant expression cassettes to drive expression of antisense nucleic acids to reduce, increase, or alter concentration and/or composition of the proteins of the present invention in a desired tissue. Thus, in some embodiments, the nucleic acid construct will comprise a promoter functional in a plant cell, such as in Zea mays, operably linked to a polynucleotide of the present invention. Promoters useful in these embodiments include the endogenous promoters driving expression of a polypeptide of the present invention.

In some embodiments, isolated nucleic acids which serve as promoter or enhancer elements can be introduced in the appropriate position (generally upstream) of a non-heterologous form of a polynucleotide so as to up or down regulate its expression. For example, endogenous promoters can be altered in vivo by mutation, deletion, and/or substitution (U.S. Pat. No. 5,565,350; PCT/US93/03868), or isolated promoters can be introduced into a plant cell in the proper orientation and distance from a gene of the present invention so as to control the expression of the gene. Gene expression can be modulated under conditions suitable for plant growth so as to alter the total concentration and/or alter the composition of the polypeptides of the present invention in plant cell.

A variety of promoters will be useful in the invention, particularly to control the expression of the ZFP and ZFP-effector fusions, the choice of which will depend in part upon the desired level of protein expression and desired tissue-specific, temporal specific, or environmental cue-specific control, if any in a plant cell. Constitutive and tissue specific promoters are of particular interest. Such constitutive promoters include, for example, the core promoter of the Rsyn7, the core CaMV 35S promoter (Odell et al. (1985) Nature 313:810-812), rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689), pEMU (Last et al. (1991) Theor. Appl. Genet. 81:581-588), MAS (Veltenet al. (1984) EMBO J. 3:2723-2730), and constitutive promoters described in, for example, U.S. Pat. Nos. 5,608,149; 5,608,144; 5,604,121; 5,569,597; 5,466,785; 5,399,680; 5,268,463; and 5,608,142.

Tissue-specific promoters can be utilized to target enhanced expression within a particular plant tissue. Tissue-specific promoters include those described by Yamamoto et al. (1997) Plant J. 12(2)255-265, Kawamata et al. (1997) Plant Cell Physiol. 38(7):792-803, Hansen et al. (1997) Mol. Gen Genet. 254(3):337), Russell et al. (1997) Transgenic Res. 6(2):157-168, Rinehart et al. (1996) Plant Physiol. 112(3):1331, Van Camp et al. (1996) Plant Physiol. 112(2):525-535, Canevascini et al. (1996) Plant Physiol. 112(2):513-524, Yamamoto et al. (1994) Plant Cell Physiol. 35(5):773-778, Lam (1994) Results Probl. Cell Differ. 20:181-196, Orozco et al. (1993) Plant Mol. Biol. 23 (6):1129-1138, Matsuoka et al. (1993) Proc Natl. Acad. Sci. USA 90(20):9586-9590, and Guevara-Garcia et al. (1993) Plant J. 4(3):495-505. Such promoters can be modified, if necessary, for weak expression.

Leaf-specific promoters are known in the art, and include those described in, for example, Yamamoto et al. (1997) Plant J. 12(2):255-265, Kwon et al. (1994) Plant Physiol. 105:357-67, Yamamoto et al. (1994) Plant Cell Physiol. 35(5):773-778, Gotor et al. (1993) Plant J. 3:509-18, Orozco et al. (1993) Plant Mol. Biol. 23(6):1129-1138, and Matsuoka et al. (1993) Proc. Natl. Acad. Sci. U.S.A .90(20):9586-9590.

Any combination of constitutive or inducible and non-tissue specific or tissue specific may be used to control ZFP expression. The desired control may be temporal, developmental or environmentally controlled using the appropriate promoter. Environmentally controlled promoters are those that respond to assault by pathogen, pathogen toxin, or other external compound (e.g., intentionally applied small molecule inducer). An example of a temporal or developmental promoter is a fruit ripening-dependent promoter. Particularly preferred are the inducible PR1 promoter, the maize ubiquin promoter, and ORS.

Thus, the present invention provides compositions, and methods for making, heterologous promoters and/or enhancers operably linked to a ZFP and ZFP-effector fusion encoding polynucleotide of the present invention.

Methods for identifying promoters with a particular expression pattern, in terms of, e.g., tissue type, cell type, stage of development, and/or environmental conditions, are well known in the art. See, e.g., The Maize Handbook, Chapters 114-115, Freeling and Walbot, Eds., Springer, New York (1994); Corn and Corn Improvement, Pedition, Chapter 6, Sprague and Dudley, Eds., American Society of Agronomy, Madison, Wis. (1988).

In the process of isolating promoters expressed under particular environmental conditions or stresses, or in specific tissues, or at particular developmental stages, a number of genes are identified that are expressed under the desired circumstances, in the desired tissue, or at the desired stage. Further analysis will reveal expression of each particular gene in one or more other tissues of the plant. One can identify a promoter with activity in the desired tissue or condition but that do not have activity in any other common tissue. Such genes can be good candidates for regulation in accordance with the methods of the invention.

In plants, further upstream from the TATA box, at positions −80 to −100, there is typically a promoter element (i.e., the CAAT box) with a series of adenines surrounding the trinucleotide G (or T) N G. J. Messing et al., in Genetic Engineering in Plants, Kosage, Meredith and Hollaender, Eds., pp. 221-227 1983. In maize, there is no well conserved CAAT box but there are several short, conserved protein-binding motifs upstream of the TATA box. These include motifs for the trans-acting transcription factors involved in light regulation, anaerobic induction, hormonal regulation, or anthocyanin biosynthesis, as appropriate for each gene.

Plant transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad Sci. USA 83:5602-5606, Agrobacterium-mediated transformation (Townsend et al., U.S. Pat. No. 5,563,055), direct gene transfer (Paszkowski et al. (1984) EMBO J. 3:2717-2722), and ballistic particle acceleration (see, for example, Sanford et al., U.S. Pat. No. 4,945,050; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al. (1988) Biotechnology 6:923-926). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) BioTechnology 6:923-926 (soybean); Finer and McMullen (199 1) In Vitro Cell Dev. Biol. 2 7P: 175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322, 783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The ZFP with optional effector domain can be targeted to a specific organelle within the plant cell. Targeting can be achieved with providing the ZFP an appropriate targeting peptide sequence, such as a secretory signal peptide (for secretion or cell wall or membrane targeting, a plastid transit peptide, a chloroplast transit peptide, a mitochondrial target peptide, a vacuole targeting peptide, or a nuclear targeting peptide, and the like. For examples of plastid organelle targeting sequences see WO00/12732. Plastids are a class of plant organelles derived from proplastids and include chloroplasts, leucoplasts, aravloplasts, and chromoplasts. The plastids are major sites of biosynthesis in plants. In addition to photosynthesis in the chloroplast, plastids are also sites of lipid biosynthesis, nitrate reduction to ammonium, and starch storage. And while plastids contain their own circular genome, most of the proteins localized to the plastids are encoded by the nuclear genome and are imported into the organelle from the cytoplasm.

The modified plant may be grown into plants by conventional methods. See, for example, McCormick et al. (1986) Plant Cell. Reports :81-84. These plants may then be grown, and either pollinated with the same transformed strain or different strains, and the resulting hybrid having the desired phenotypic characteristic identified. Two or more generations may be grown to ensure that the subject phenotypic characteristic is stably maintained and inherited and then seeds harvested to ensure the desired phenotype or other property has been achieved.

Assays to determine the efficiency by which the modulation of the target gene or protein of interest occurs are known. In brief, in one embodiment, a reporter gene such as glucuronidase (GUS), chloramphenicol acetyl transferase (CAT), or green fluorescent protein (GFP) is operably linked to the target gene sequence controlling promoter, ligated into a transformation vector, and transformed into a plant or plant cell.

ZFPs useful in the invention comprise at least one zinc finger polypeptide linked via a linker, preferably a flexible linker, to at least a second DNA binding domain, which optionally is a second zinc finger polypeptide. The ZFP may contain more than two DNA-binding domains, as well as one or more regulator domains. The zinc finger polypeptides of the invention can be engineered to recognize a selected target site in the gene of choice. Typically, a backbone from any suitable Cys₂His₂-ZFP, such as SPA, SPIC, or ZIF268, is used as the scaffold for the engineered zinc finger polypeptides (see, e.g., Jacobs, EMBO J. 11:45 07 (1992); Desjarlais & Berg, Proc. Natl. Acad. Sci. USA 90:2256-2260 (1993)). A number of methods can then be used to design and select a zinc finger polypeptide with high affinity for its target. A zinc finger polypeptide can be designed or selected to bind to any suitable target site in the target gene, with high affinity.

As to amino acid and nucleic acid sequences, individual substitutions, deletions or additions that alter, add or delete a single amino acid or nucleotide or a small percentage of amino acids or nucleotides in the sequence create a “conservatively modified variant,” where the alteration results in the substitution of an amino acid with a chemically similar amino acid. Conservative substitution tables providing functionally similar amino acids are well known in the art. Such conservatively modified variants are in addition to and do not exclude polymorphic variants and alleles of the invention.

The following groups each contain amino acids that are conservative substitutions for one another: 1) Alanine (A), Glycine (G); 2) Serine (S), Threonine (T); 3) Aspartic acid (D), Glutamic acid (E); 4) Asparagine (N), Glutamine (Q); 5) Cysteine (C), Methionine (M); 6) Arginine (R), Lysine (K), Histidine (H); 7) Isoleucine (1), Leucine (L), Valine (V); and 8) Phenylalanine (F), Tyrosine (Y), Tryptophan (W). (see, e.g., Creighton, Proteins (1984) for a discussion of amino acid properties).

Thus, the invention contemplates gene regulation which may be tissue specific or not, inducible or not, and which may occur in plant cells either in culture or in intact plants. Useful activation or repression levels can vary, depending on how tightly the target gene is regulated, the effects of low level changes in regulation, and similar factors. Desirably, the change in gene expression is modified by about 1.5-fold to 2-fold; more desirably, about 3-fold to 5-fold; preferably about 8- to 10- to 15-fold; more preferably 20- to 25- to 30-fold; most preferably 40-, 50-, 75-, or 100-fold, or more. In this context, modification of expression level refers to either activation or repression of normal levels of gene expression in the absence of the activator/repressor activity. Measured activity of a particular ZFP-effector fusion varied somewhat from plant to plant as a result of the effect of the chromosomal location of integration of the ZFP-effector construct.

Typical vectors useful for expression of genes in higher plants are well known in the art and include vectors derived from the tumor-inducing (Ti) plasmid of Agrobacterium tumefaciens described by Rogers et al., Meth. in Enzymol., 153:253-277 (1987). These vectors are plant integrating vectors in that on transformation, the vectors integrate a portion of vector DNA into the genome of the host plant. Exemplary A. tumefaciens vectors useful herein are plasmids pKYLX6 and pKYLX7 of Schardl et al., Gene, 6 1: 1-11 (1987) and Berger et al., Proc. Natl. Acad. Sci. U.S.A., 86:8402-8406 (1989). Another useful vector is plasmid pBI101.2.

The method of the invention is particularly appealing to the plant breeder because it has the effect of providing a dominant trait, which minimizes the level of crossbreeding necessary to develop a phenotypically desirable species which is also commercially valuable. Typically, modification of the plant genome by conventional methods creates heterozygotes where the modified gene is phenotypically recessive. Crossbreeding is required to obtain homozygous forms where the recessive characteristic is found in the phenotype. This crossbreeding is laborious and time consuming. The need for such crossbreeding is eliminated in the case of the present invention which provides an immediate phenotypic effect.

In one embodiment, the ZFP can be designed to bind to non-contiguous target sequences. For example, a target sequence for a six-finger ZFP can be a ten base pair sequence (recognized by three fingers) with intervening bases (that do not contact the zinc finger nucleic acid binding domain) between a second ten base pair sequence (recognized by a second set of three fingers). The number of intervening bases can vary, such that one can compensate for this intervening distance with an appropriately designed amino acid linker between the two three-finger parts of ZFP. A range of intervening nucleic acid bases in a target binding site is preferably 20 or less bases, more preferably 10 or less, and even more preferably 6 or less bases. Of course, the linker maintains the reading frame between the linked parts of ZFP protein.

A minimum length of a linker is the length that would allow the two zinc finger domains to be connected without providing steric hindrance to the domains or the linker. A linker that provides more than the minimum length is a “flexible linker.” Determining the length of minimum linkers and flexible linkers can be performed using physical or computer models of DNA-binding proteins bound to their respective target sites as are known in the art.

The six-finger zinc finger peptides can use a conventional “TGEKP” linker to connect two three-finger zinc finger peptides or to add additional fingers to a three-finger protein. Other zinc finger peptide linkers, both natural and synthetic, are also suitable. In addition to such linkers, the domains can be covalently joined with from 1 to 10 additional amino acids. Such additional amino acids may be most beneficial when used after every third zinc-finger domain in a multifinger ZFP.

A useful zinc finger framework is that of Berg (see Kim et al., Nature Struct. Biol. 3:940-945, 1996; Kim et al., J. Mol. Biol. 252:1-5, 1995; Shi et al., Chemistry and Biology 2:83-89, 1995), however, others are suitable. Examples of known zinc finger nucleotide binding polypeptides that can be truncated, expanded, and/or mutagenized according to the present invention in order to change the function of a nucleotide sequence containing a zinc finger nucleotide binding motif includes TFIIIA and Zif268. Other zinc finger nucleotide binding proteins will be known to those of skill in the art. The murine Cys₂-His₂ ZFP Zif268 is structurally the most well characterized of the ZFPs (Pavletich and Pabo, Science 252:809-817 (1991), Elrod-Erickson et al. (1996) Structure (London) 4, 1171-1180, Swirnoff et al. (1995) Mol, Cell. Biol. 15:2275-2287). DNA recognition in each of the three zinc finger domains of this protein is mediated by residues in the N-terminus of the alpha-helix contacting primarily three nucleotides on a single strand of the DNA. The operator binding site for this three finger protein is 5′-GCGTGGGCG-′3. Structural studies of Zif268 and other related zinc finger-DNA complexes (Elrod-Erickson, M., Benson, T. E. & Pabo, C. 0. (1998) Structure (London) 6, 451-464, Kim and Berg, (1996) Nature Structural Biology 3, 940-945, Pavletich and Pabo, (1993) Science 261, 1701-7, Houbaviy et al. (1996) Proc Natl. Acad. Sci. USA 93, 13577-82, Fairall et al. (1993) Nature (London) 366, 483-7, Wuttke et al. (1997) J. Mol. Biol. 273, 183-206., Nolte et al. (1998) Proc. Nad. Acad. Sci. U.S.A. 95, 2938-2943, Narayan, et al. (1997) J. Biol. Chem. 272, 7801-7809) have shown that residues from primarily three positions on the a-helix, −1, 3, and 6, are involved in specific base contacts. Typically, the residue at position −1 of the a-helix contacts the 3′ base of that finger's subsite while positions 3 and 6 contact the middle base and the 5′ base, respectively.

Any suitable method of protein purification known to those of skill in the art can be used to purify the ZFPs of the invention (see Ausubel, supra, Sambrook, supra). In addition, any suitable host can be used, e.g., bacterial cells, insect cells, yeast cells, mammalian cells, and the like.

In an embodiment, longer genomic sequences are targeted using multi-finger ZFPs linked to other multi-fingered ZFPs using flexible linkers including, but not limited to, GGGGS, GGGS and GGS (these sequences can be part of the 1-10 additional amino acids in the ZFPs of the invention; SEQ ID NO:23, residues 2-5 of SEQ ID NO:23; and residues 3-5 of SEQ ID NO:23, respectively). Non-palindromic sequences may be targeted using dimerization peptides such as acidic and basic peptides, optionally in combination with a flexible linker, in which ZFPs are attached to the acidic and basic peptides (effector domain-acidic or basic peptide-ZFP). At the other end of the acidic and basic peptides are effector peptides, such as activation domains. These domains may be assembled in any order. For example, the arrangement of ZFP-effector domain-acidic or basic peptide is also within the scope of the present invention. In addition, it is not required that a zinc finger peptide be attached to both the acidic and basic peptides; one or the other or both is within the scope of the invention. The need for two ZFPs will depend upon the affinity of the first ZFP. These constructs can be used for combinatorial transcriptional regulation (Briggs, et al.) using the heterodimer described above. The protein only dimerizes when both halves are expressed. Thus, activation or inhibition of gene expression will only occur when both halves of the protein are expressed in the same cell at the same time. For example, two promoters may be used for expression in plants, one tissue-specific and one temporal. Activation of gene expression will only occur when both halves of the heterodimer are expressed.

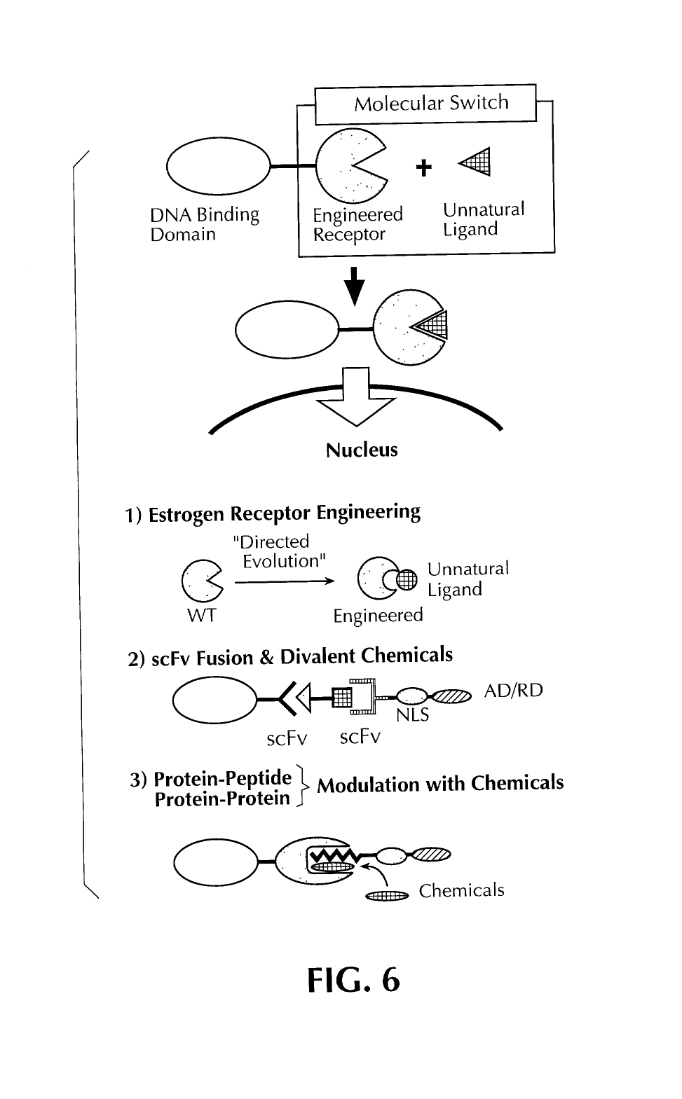

The present invention also relates to “molecular switches” or “chemical switches” which are used to promote translocation of ZFPs generated according to the recognition code of the present invention to the nucleus to promote transcription of a gene of interest. The molecular switch is, in one embodiment, a divalent chemical ligand which is bound by an engineered receptor, such as a steroid hormone receptor, and which is also bound by an engineered ZFP (FIG. 6). The receptor-ligand-zinc finger complex enters the nucleus where the ZFP binds to its target site. An example is a complex comprising a ZFP linked by a divalent chemical ligand having moieties A and B to a nuclear localization signal which is operably linked to an effector domain such as an activation domain (AD) or repression domain (RD). A construct encoding a ZFP and an antibody specific for moiety A (or an active fragment of such antibody) is expressed in a cell. A second construct, encoding an engineered nuclear localization signal/effector domain and an antibody specific for moiety B (or an active fragment of such antibody) is separately expressed in the same cell. Upon addition to the cell of the divalent chemical that includes moiety A and moiety B linked together, the affinity of each separately expressed fusion protein for either moiety A or moiety B mediates formation of a complex in which the engineered ZFP is physically linked to the nuclear localization and effector domains. This embodiment permits very specific inducibility of localization of the complex to the nucleus by dosing cells with the divalent chemical. Numerous possibilities exist for moieties A and B. The criteria are that the moiety is sufficiently antigenic to allow selection of a monoclonal antibody specific for that moiety, and that the two moieties, linked together, form a compound that can enter and act within a cell to mediate formation of the complex. In one embodiment, moiety A can have a structure, for example, as depicted below:

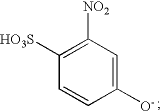

moiety B can have a structure, for example, as depicted below:

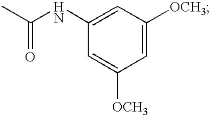

and moieties A and B can be linked by a linker of any suitable length, having units such as those depicted below:

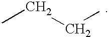

Any compound capable of entry into cell and having moieties against which antibodies can be raised is suitable for this aspect of the invention. This embodiment of the invention permits sequence-specific localization of the effector domain to allow it to act on the selected promoter, causing an alteration of gene expression in the cell which can, for example, produce a desired phenotype. In the absence of the divalent chemical, such a phenotype is not manifest, because the site specificity conferred by the ZFP is not joined to the nuclear localization and effector activity of the engineered effector protein. Accordingly, induction of the site specific effector activity is achieved by addition of the divalent chemical.

In a preferred embodiment, a chemical switch is used which is a divalent chemical comprising two linked compounds. These compounds may be any compounds to which antibodies can be raised linked by a short linker, for example, CH₂CH₂. In one preferred embodiment, a single chain antibody (e.g., a single chain Fᵥ (scFᵥ)) binds to one portion of the divalent chemical to link it to a ZFP. The other portion of the divalent chemical binds to a second single chain antibody, for example a single chain Fᵥ (scFᵥ), which recognizes and binds to a nuclear targeting sequence (e.g., nuclear localization signal) which is operably linked to an effector domain, preferably an activator or repressor domain (FIG. 6). Thus, translocation of the ZFP into the nucleus will only occur in the presence of the divalent chemical. In an alternative embodiment, the effector domain is bound to the ZFP which is in turn bound to a single chain antibody. However, because the effector-ZFP-antibody complex may diff-use into the nucleus in the absence of the divalent chemical, it is preferable that the ZFP and effector domains are on separate proteins. Even if the ZFP-antibody diffuses into the nucleus, it would at worst be a negative regulator, not an activator, until the chemical is present. This is also not as preferred because it is more preferable to manipulate the translocation of both the ZFP and effector domain.

The chemical switch embodiments of the invention are also applicable to engineering other useful inducible gene expression systems. For example, using this approach, artificial defense mechanisms can be engineered into a plant. When pathogens infect plants, small molecule “elicitors” are often produced. The antibodies in the molecular switch system can thus be specific to such elicitor compounds, such that only in the presence of elicitors is the inducible gene expression complex formed, allowing an engineered response to the pathogenic infection. In this manner, plant defense genes can be directly and immediately activated without influence of “suppressors” produced by pathogens when pathogens infect the plant. In a preferred embodiment, two scFvs (scFv-1 and scFv-2) are produced. Each scFv recognizes a different part of an elicitor (that is, different epitopes on the elicitor molecule). The zinc finger/scFv-1 fusion protein and the NLS-AD-scFv-2 fusion protein bind to the elicitor, creating the gene activation complex capable of localization to the nucleus, and plant defense genes are selectively activated based on the design of the ZFP. By this approach, plant defense genes are only activated in the presence of the pathogen.

Another embodiment of the invention relating to combinatorial transcriptional regulation involves the S-tag, S-protein system. The S-tag is a short peptide (15 amino acids) and S-protein is a small protein (104 amino acids). The affinity of the S-tag and S-protein complex is high (Kd=1 nM). The S-tag/S-protein system can be used in a chemical switch system. In this embodiment, the S-tag is conjugated to a ZFP, and the S-protein is conjugated to a nuclear localization signal (NLS) which is conjugated to an activation domain (AD) or to a repressor. The S-tag-zinc finger and S-protein-NLS-AD constructs are expressed using two different promoters, resulting in formation of a zinc finger-S-tag-S-protein-NLS-AD complex. The chemical switch involves the use of S-tag and S-protein mutants which cannot interact unless a small molecule or chemical is present to link the S-tag and S-protein together. These small molecules can also be used to disrupt wild type S-tag-S-protein interaction.

In another embodiment of the invention, ZFPs or fusion proteins comprising zinc finger domains and single strand DNA binding protein (SSB) are used to inhibit viral replication. Geminivirus replication can be inhibited using zinc finger domains or zinc finger-SSB fusion proteins which are targeted to “direct repeat” sequences or “stem-loop” structures which are conserved in all gemini viruses, which are nicked to provide a primer for rolling circle replication of the viral genome. For example, AL1 is a tobacco mosaic virus (TMV) site-specific endonuclease which binds to a specific site on TMV. After binding, AL1 cleaves the viral DNA in the stem-loop to begin rolling circle viral replication. A ZFP or zinc finger-SSB fusion protein is engineered using the recognition code of the invention, such that the SSB portion binds to the cleavage site, and the zing finger domain binds adjacent to this site. Alternatively, a ZFP alone is used which is designed to bind to the AL1 binding or cleavage site, thus preventing AL1 from binding to its binding site or to the stem-loop structure. Thus, ZFPs competitively inhibit binding of AL1 to its target site. These types of ZFPs or zinc-finger SSB fusion proteins can be designed to target any desired binding site in any DNA or RNA virus which is involved in viral replication. In addition, because the stem-loop structure is conserved in all geminiviruses, the nick site of all such viruses can be blocked using similar ZFPs or zinc finger-SSB fusions.

Another embodiment of the invention relates to methods for detecting an altered zinc finger recognition sequence. In this method a nucleic acid containing the zinc finger recognition sequence of interest is contacted with a ZFP of the invention that is specific for the sequence and conjugated to a signaling moiety, the ZFP present in an amount sufficient to allow binding of the ZFP to its recognition (i.e., target) sequence if said sequence was unaltered. The extents of ZFP binding is then determine by detecting the signaling moiety and thereby ascertain whether the normal level of binding to the zinc finger recognition sequence has changed. If the binding is diminished or abolished relative to binding of said ZFP to the unaltered sequence, then the recognition sequence has been altered. This method is capable of detecting altered zinc finger recognition site in which a mutation (substitution), insertion or deletion of one or more nucleotides has occurred in the site. The method is useful for detecting single nucleotide polymorphisms (SNPs).

Any convenient signaling moiety or system can be used. Examples of signaling moieties include, but are not limited to, dyes, biotin, radioactive labels, streptavidin an marker proteins. Many marker proteins are known, but not limited to, β-galactosidase, GUS (β-glucuronidase), green fluorescent proteins, including fluorescent mutants thereof which have altered spectral properties (i.e., exhibit blue or yellow fluorescence, horse radish peroxidase, alkaline phosphatase, antibodies, antigens and the like.

In addition, the present invention contemplates a method of diagnosing a disease associated with abnormal genomic structure. Examples of such diseases are those where there is an increased copy number of particular nucleic acid sequences. For example, the high copy number of the indicated sequences is found in persons with the indicated disease relative to the copy number in a healthy individual: (CAG)ₙ for Huntington disease, Friedreich ataxia; (CGG)ₙ for Fragile X site A; (CCG)ₙ for Fragile X site E; and (CTG)ₙ for myotonic dystrophy.

This method comprises (a) isolating cells, blood or a tissue sample from a subject; (b) contacting nucleic acid in or from the cells, blood or tissue sample with a ZFP of the invention (with specificity for the target of the disease in question) linked to a signaling moiety and, also, optionally, fused to a cellular uptake domain; and (c) detecting binding of the protein to the nucleic acid to thereby make a diagnosis. If necessary, the amount of binding can be quantitated and this may aid is assessing the severity or progression of the disease in some cases. The method can be performed by fixing the cells, blood or tissue appropriately so that the nucleic acids are detected in situ or by extracting the nucleic acids from the cells, blood or tissue and then performing the detection and optional quantitation step.

VII. Pharmaceutical Formulations

Therapeutic formulations of the ZFPs, fusion proteins or nucleic acids encoding those ZFPs or fusion proteins of the invention are prepared for storage by mixing those entities having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the polypeptide variant, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Those of skill in the art can readily determine the amounts of the ZFPs, fusion proteins or nucleic acids encoding those ZFPs or fusion proteins to be included in any pharmaceutical composition and the appropriate dosages for the contemplated use.

Throughout this application, various publications, patents, and patent applications have been referred to. The teachings and disclosures of these publications, patents, and patent applications in their entireties are hereby incorporated by reference into this application.

It is to be understood and expected that variations in the principles of invention herein disclosed in exemplary embodiments may be made by one skilled in the art and it is intended that such modifications, changes, and substitutions are to be included within the scope of the present invention.

### EXAMPLE 1

### Design of ZFP Using Recognition Code

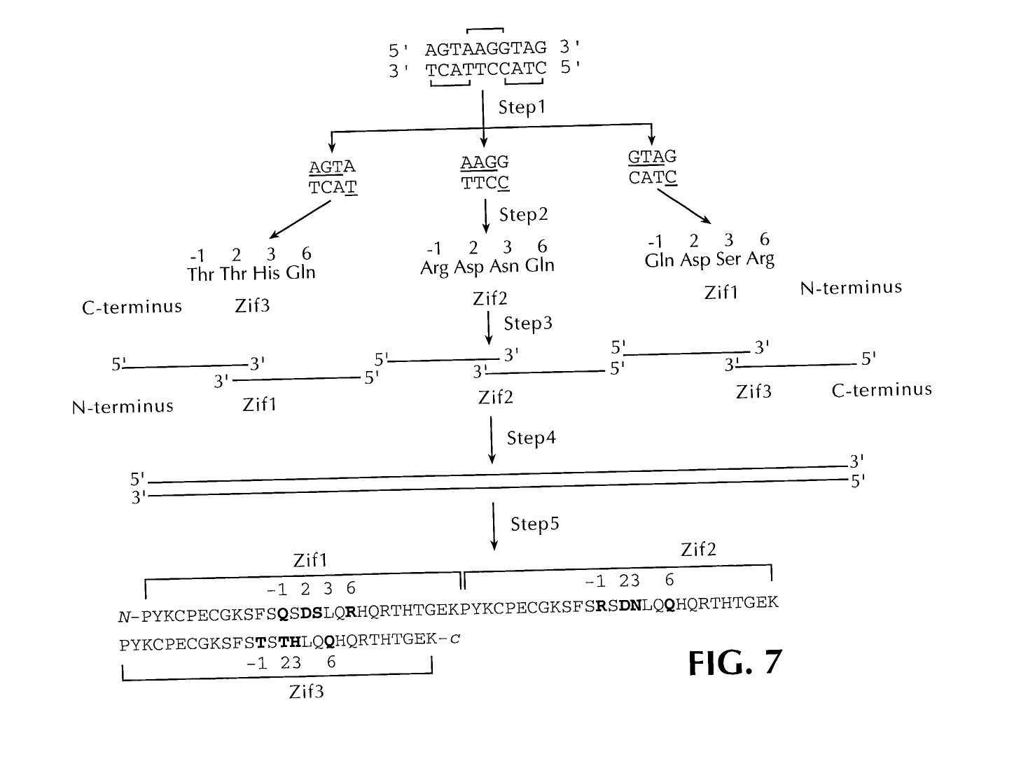

To confirm the amino acid-base contacts shown in Table 1, a ZFP targeting the AL1 binding site in the tomato golden mosaic virus genome was designed. As shown in FIG. 7, the target site, 5′-AGTAAGGTAG-3′ (SEQ ID NO: 14), was divided into three regions each having four DNA base pairs (Step 1). These regions were overlapping in that the fourth base of the first region became the first base of the second region, and the fourth base of the second region became the first base of the third region. Thus, three zinc fingers are used to target a 10 base pair region of nucleic acid. Next, four amino acids per four DNA base pairs were chosen from the table for use with the Sp1C-domain 2 frame work described by Berg (Step 2). Amino acids other than those at positions −1, 2, 3 and 6 were not modified. DNA oligomers corresponding to the peptide sequence were synthesized by standard methods using a DNA synthesizer (Step 3). These three zinc finger domains were then assembled by one polymerase chain reaction (PCR) to construct the ZFP targeting the AL1 site (Step 4). The DNA fragments were cloned into the EcoRI/HindIII sites of a pET21-a vector (Novagen). The resulting plasmids were introduced into E. coli BL21(DE3)pLysS for protein overexpression and purified by cation exchange column chromatography (Step 5). A 60 mL culture was grown to OD₆₀₀=0.75 at 37° C., induced with 1 mM IPTG for 3 hours, and lysed by freeze thaw in cold lysis buffer (100 mM Tris-HCl, pH 8.0, 1 M NaCl, 5 mM dithiothreitol (DTT), 1 mM ZnCl₂. After treatment with polyethyleneimine (pH 7, 0.6%) and precipitation with 40% (NB₄)₂SO₄, the resulting pellet was redissolved in 50 mM Tris-HCl, pH 8.0, 100 mM NaCl, 5 mM DTT, 0.1 mM ZnCl₂ and purified using a Bio-Rex 70 cation exchange column, eluting with 0.3 mM NaCl buffer. All purified proteins were >95% homogeneous as judged by sodium dodecyl sulfate-polyacrylamide gel electrophoresis (SDS-PAGE).

### EXAMPLE 2

### Determination of Affinity of ZFP for Target Sequence

To test the affinity of the synthesized ZFP for the target sequence, a gel shift experiment was performed using an AL1 target polynucleotide (5′-TATATATAAGTAAGGTAGTATATATA-3′; SEQ ID NO: 24). As a positive control, the ZFP Zif268 and a target polynucleotide for this protein (5′-TATATATAGCGTGGGCGTTATATATA-3′; SEQ ID NO: 25) were also used. The targeting site of each ZFP is underlined. The concentrations of AL1 ZIF; in the assay were 0, 14, 21, 28, 35, 70 and 88 mM. The concentrations of Zif268 were 2.6, 3.3, 6.6, 13 and 20 μM. Prior to the assay, target polynucleotides were labeled at the 5′-end with [γ-³²P]ATP. ZFPs were preincubated on ice for 40 minutes in 10 μL of 10 mM Tris-HCl, pH 7.5, 100 mM NaCl, 1 mM MgCl₂, 0.1 mM ZnCl₂, 1 mg/ml BSA, 10% glycerol containing the end-labeled probe (1 pmol). Poly (dA-dT)₂ was then added, and incubation was continued for 20 minutes before electrophoresis on a 6% nondenaturing polyacrylamide gel (0.5×tris-borate buffer) at 140 volts for 2 hours at 4° C. half-maximal binding of the AL1 and Zif268 ZFP was observed at 18 nM and 4 nM, respectively. The affinity of the AL1 ZFP for its target sequence is also comparable to the ZFPs selected using phage display (30-40 nM, PCT WO95/19431; Liu et al., Proc. Natl. Acad. Sci. U.S.A. 94:5525-5530, 1997).

### EXAMPLE 3

### Determination of DNA Base Specificity

To determine DNA base specificity, the following study was conducted. Based on FIG. 3, the aspartic acid at position 2 in the first zinc finger domain is expected to bind to the cytosine at the 3′ end of the 4 base pair region. A gel shift assay was performed as described above, using the AL1 ZFP (14, 21 and 35 nM concentrations) and the following end-labeled polynucleotides: 5′-(TA)₄AGTAAGGTAG(TA)₄ (SEQ ID NO: 26); 5′-(TA)₄AGTAAGGTAA(TA)₄ (SEQ ID NO: 27); 5′-(TA)₄AGTAAGGTAT(TA)₄ (SEQ ID NO: 28); and 5′-(TA)₄AGTAAGGTAC(TA)₄ (SEQ ID NO: 29). SEQ ID NO: 24 is the wild-type target sequence having a G at the 3′ end of the 10 base pair sequence. The other three polynucleotides have point mutations at this position (A, T and C in SEQ ID NOS: 27, 28, and 29, respectively—base is underlined). Significant binding of the AL1 ZFP only occurred when the protein was incubated with SEQ ID NO: 27. Very little binding to SEQ ID NOS: 27, 28, or 29 was observed, thus confirming the specific interaction of aspartic acid at position 2 with guanine at the 3′ end of the four base pair region.

### EXAMPLE 4

### Recognition Code

The complete recognition code is confirmed by individually screening amino acids at positions −1, 2, 3 and 6 of a ZFP. For example, in the screening of amino acids at position 2, the protein comprising three zinc finger domains:

| | |
|---|---|
| PYKCPECGKSFSDSXALQRHQRTHTGEKPYKCPECGKSFSQSSNLQKHQRTHTGEKP | (SEQ ID NO:30) |
| YKCPECGKSFSRSDHLQRHQRTHTGEK | |

is used for the screening (X, underlined at position 2, is mutated). The first zinc finger domain is used to identify DNA base specificity at position 2 because the domain (Asp, Ala and Arg at positions −1, 3, and 6, respectively) is known to bind to DNA randomly. The degenerate DNA probes 5′-GGGGAANNNY-3′ (N=equimolar mixture of G, A, T, or C; Y=G, A, T or C; SEQ ID NO: 31) are used in order to identify the DNA base specificity of amino acids at position 2 without the influence of DNA base-amino acid interactions at other positions.

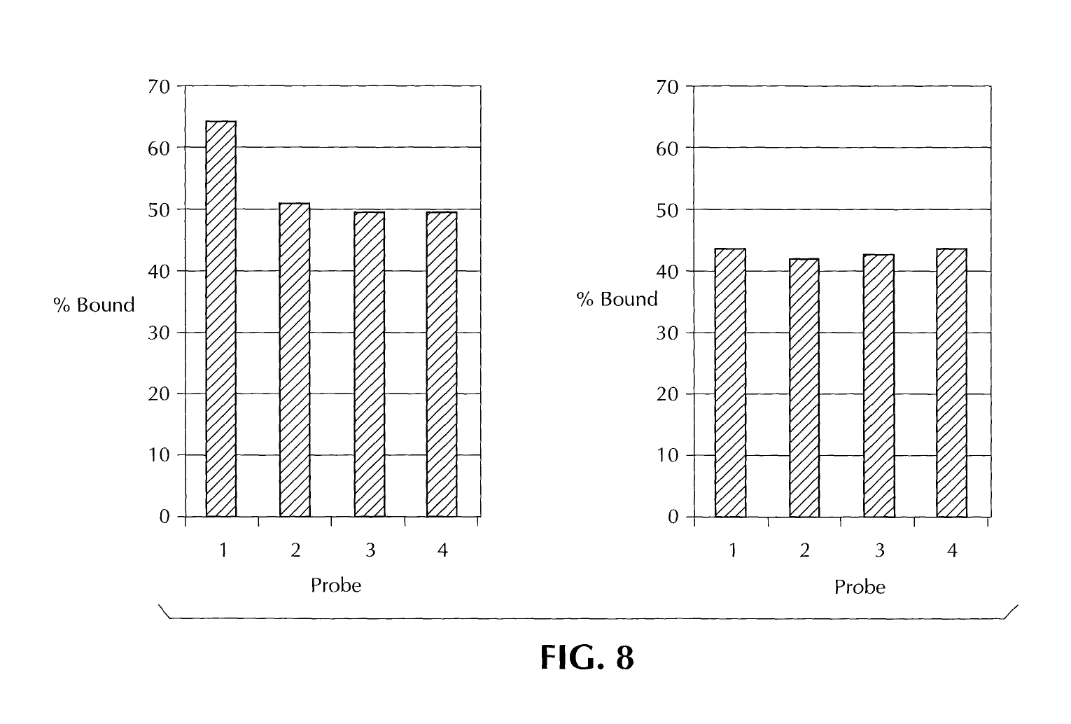

The Asp and Gly mutant proteins were prepared and the DNA base specificity was investigated using the gel shift assay. The following ³²P-labeled duplexes were used: 5′-(TA)₄GGGGAANNNG(TA)₄ (1) (SEQ ID NO: 32); 5′-(TA)₄GGGGAANNNA(TA)₄ (2) (SEQ ID NO: 33); 5′-(TA)₄GGGGAANNNT(TA)₄ (3) (SEQ ID NO: 34); and 5′-(TA)₄GGGGAANNNC(TA)₄ (4) (SEQ ID NO: 35). As shown in FIG. 8, the Asp mutant preferentially bound to 5′-GGGGAANNNG-3′ (Probe 1; bases 9-18 of SEQ ID NO: 32). The mutation from Asp to Gly resulted in loss of selectivity as shown in FIG. 8. This shows that aspartic acid at position 2 independently recognizes the cytosine base at the 4^{th} position in the DNA target. The recognition of the cytosine base at the 4^{th} position by the aspartic acid at position 2, which is predicted in Table 1, was experimentally confirmed. The complete recognition code is confirmed by repeating similar experiments with other amino acids.

### EXAMPLE 5

### Engineering of Transposases and Transposition Assay

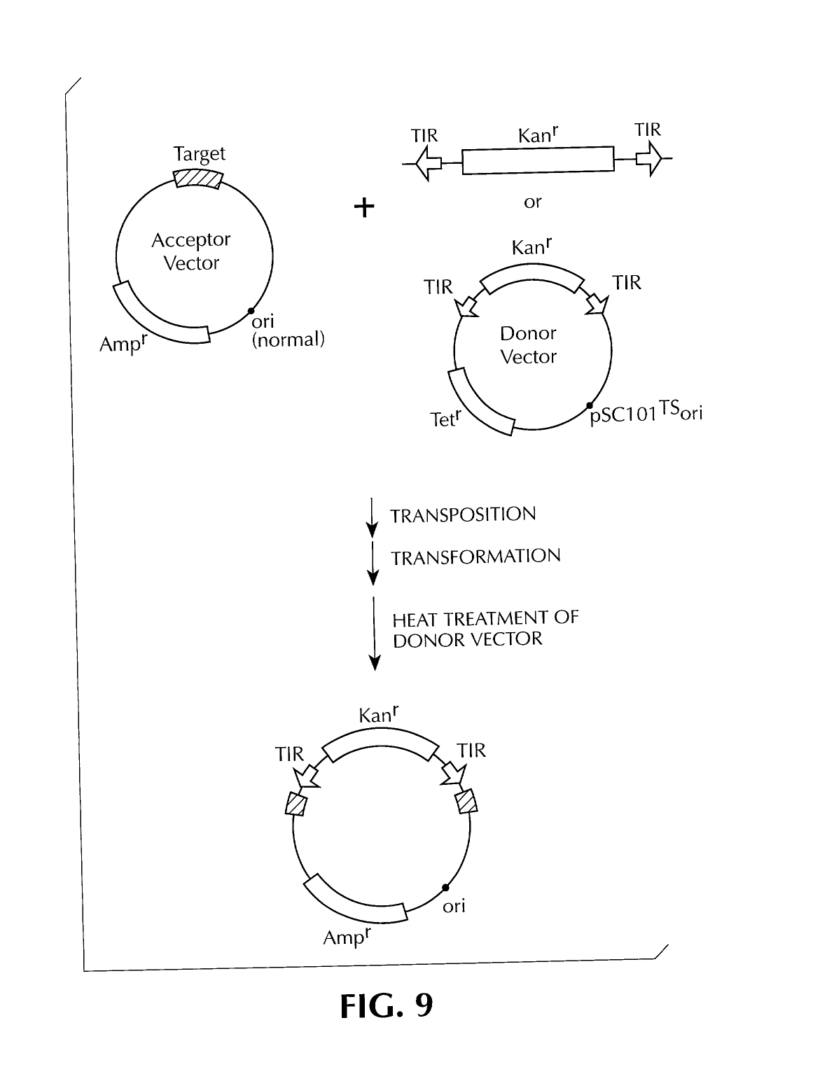

The C. elegans transposase Tc1 is useful to demonstrate creation of a site-specific, genetic knock-in using a ZFP fused to Tc1. The transposition method is summarized in FIG. 9. A marker fragment or plasmid containing the homogeneous TIRs is used which contains a selectable marker gene (e.g., kanamycin resistance) between the TIRs. An acceptor vector comprising a target region (e.g., 1 or 2 Zif268 binding sites), a normal origin of replication and ampicillin resistance is combined with the TIR-kanamycin-TIR linear fragment, or with a donor vector comprising this construct, tetracycline resistance and a pSC101^{TS} ori temperature-sensitive origin of replication. In this case the TIRs are the same (homoassay); however, a similar assay can be done using different TIRs and different TIR binding domains (such as that from C. elegans transposase Tc30)(heteroassay). The transposition reaction is performed using the ZFP-transposase fusion protein followed by E. coli transformation and, in the case of the donor vector, heat treatment to eliminate the unreacted donor vector, resulting in a vector in which the TIR-kanamycin-TIR construct has been inserted into the Zif268 target site of the acceptor vector. Transposition efficiency is determined by comparing the titer of ampicillin resistant E. coli to ampicillin-kanamycin resistant E. coli.

### EXAMPLE 6

### General Scheme for Producing Three-Finger ZFPs

Each finger of the ZFP was designed to have the same frame work sequence, PYKCPECGKSFSXSXXLQXHQRTHTGEK (SEQ ID NO: 13), wherein X, at positions −1, 2, 3 and 6, are determined according to the zinc finger recognition code of Table 1 and the desired target sequence. The DNA for each finger was designed to enable the assembly of DNA encoding three zinc finger domains in correct orientation by PCR. Three pairs of DNA oligonucleotides were synthesized, each pair being two overlapping oligomers coding for one specific finger domain as follows:

| | |
|---|---|
| First Zinc Finger Domain (Zif-1) | |
| Zif-1, sense-oligomer (Primer 1) | |
| 5′-GGGGAGAAGCCGTATAAATGTCCGGAATGTGGTAAAAGTTTTAGCNNNAGCNNNNNNTTG-3′ | (SEQ ID NO:36) |
| Zif-1, antisense-oligomer (Primer 2) | |
| 5′-TTTGTATGGTTTTTCACCGGTATGGGTACGCTGATGNNNCTGCAANNNNNNGCTNNNGCT-3′ | (SEQ ID NO:37) |
| Second Zinc Finger Domain (Zif-2) | |
| Zif-2, sense-oligomer (Primer 3) | |
| 5′-GGTGAAAAACCATACAAATGTCCAGAGTGCGGCAAATCTTTCTCTNNNTCTNNNNNNCTT-3′ | (SEQ ID NO:38) |
| Zif-2, antisense-oligomer (Primer 4) | |
| 5′-CTTGTAAGGCTTCTCGCCAGTGTGAGTACGCTGATGNNNCTGAAGNNNNNNAGANNNAGA-3′ | (SEQ ID NO:39) |
| Third Zinc Finger Domain (Zif-3) | |
| Zif-3, sense-oligomer (Primer 5) | |
| 5′-GGCGAGAAGCCTTACAAGTGCCCTGAATGCGGGAAGAGCTTTAGTNNNAGTNNNNN-3 | (SEQ ID NO:40) |
| Zif-3, antisense-oligomer (Primer 6) | |
| 5′-CTTCTCCCCCGTGTGCGTGCGTTGGTGNNNTTGTAANNNNNNACTNNNACTAAAG-3′ | (SEQ ID NO:41) |

In each of these DNA-encoding finger domains, N is G, A, T, or C.

The 18 nucleotides at the 3′ end of each DNA oligonucleotide in each pair are complementary to each other. The first two DNA oligonucleotide sequences of each pair are annealed and filled in by Klenow Fragment to produce a DNA fragment coding one finger. Moreover, in order to ensure correct orientation of the zinc finger domains, the 18-bp at the 5′ end of the Zif-2 DNA fragment is complementary to 18-bp at 3′ end of Zif-1, and 18-bp of 3′ end of Zif-2 to 18-bp at 5′ end of Zif-3. Therefore, these three finger DNAs can be assembled in correct orientation by specific primers, OTS-007 and OTS-008.

| | |
|---|---|
| OTS-007: | |
| 5′-GGGCCCGGTCTCGAATTCGGGGAGAAGCCGTATAAATGTCCGGAA-3′ | (SEQ ID NO:42) |
| OTS-008: | |
| 5′-CCCGGGGGTCTCAAGCTTTTACTTCTCCCCCGTGTGCGTGCGTTGGTG-3′ | (SEQ ID NO:43) |

### EXAMPLE 7

### 3-finger ZFP for the L1 Site of Beet Curly Top Virus (BCTV)

Based on the target DNA sequence of BCTV, 5′-TTGGGTGCTC-3′ (SEQ ID NO: 44), a DNA encoding the 3-finger protein was designed. Six oligonucleotides were synthesized as shown:

| | |
|---|---|
| Zif-1, sense-oligomer (OTS-254) | |
| 5′-GGGGAGAAGCCGTATAAATGTCCGGAATGTGGTAAAAGTTTTAGCACCAGGAGCGATTTG-3′ | (SEQ ID NO:45) |
| Zif-1, antisense-oligomer (OTS-255) | |
| 5′-TTTGTATGGTTTTTGACCGGTATGGGTACGCTGATGACGCTGCAAATCGCTGCTGGTGCT-3′ | (SEQ ID NO:46) |
| Zif-2, sense-oligomer (OTS-256) | |
| 5′-GGTGAAAAACCATACAAATGTCCAGAGTGCGGCAAATCTTTCTCTACCTCTGATCATCTT-3′ | (SEQ ID NO:47) |
| Zif-2, antisense-oligomer (OTS-257) | |
| 5′-CTTGTAAGGCTTCTCGCCAGTGTGAGTACGCTGATGACGCTGAAGATGATCAGAGGTAGA-3′ | (SEQ ID NO:48) |
| Zif-3, sense-oligomer (OTS-258) | |
| 5′GGCGAGAAGCCTTACAAGTGCCCTGAATGCGGGAAGAGCTTTAGTCGTAGTGATAG-3′ | (SEQ ID NO:49) |
| Zif-3, antisense-oligomer (OTS-259) | |
| 5′-CTTCTCCCCCGTGTGCGTGCGTTGGTGGGTTTGTAAGCTATCACTACGACTAAAG-3′ | (SEQ ID NO:50) |

1) Annealing

5 μl of both OTS-254 and OTS-256, both OTS-256 and OTS-257, and both OTS-258 and OTS-259 (all, 100 pmol/μl) was added to 10 μl of TEN buffer (20 mM Tris-HCl (pH 8.0)/2 mM EDTA/200 mM NaCl), respectively, incubated at 95° C. for 5 min, and then left in the heating block at room temperature until it reached room temperature.

1 μl of each annealed sample was incubated at 37° C. for 1 hr in 20 μl of the reaction buffer containing 5 units of Klenow Fragment and 0.25 mM of dNTP mixture. After incubation, 5 μl of H₂O was added to each reaction mixture to adjust the DNA concentration to 1 pmol/μl.

2) PCR Assembly

The following was mixed and PCR was performed:

| H₂O | | 36.5 | μl |
|---|---|---|---|
| 10 X Vent Buffer | | 5 | μl |
| dNTP mixture (2.5 mM each) | | 4 | μl |
| OTS-007 (100 pmol/μ) | | 0.5 | μl |
| OTS-008 (100 pmol/μ) | | 0.5 | μl |
| Filled-in Samples: | OTS-254/255 | 1 | μl |
| | OTS-256/257 | 1 | μl |
| | OTS-258/259 | 1 | μl |

| | | |
|---|---|---|
| Vent DNA polymerase | 0.5 | μl |

The reaction product was analyzed on a 2% agarose gel and produced the expected 300-bp DNA fragment as the single major band. After cloning of this product into a pET-21a vector, DNA sequencing confirmed that these three DNA fragments were assembled in the correct orientation to produce the artificial ZFP targeting the L1 binding site of BCTV. No random assembled product was observed.

### EXAMPLE 8

### Assembly of 5-finger Domains

A 5-finger ZFP was designed to target the 16-bp sequence of the promoter of Arabidopsis DREB1A gene.

1) Preparation of DNAs encoding a 3-finger and a 2-finger ZFPs with PCR primers containing the BsaI restriction site

The sequence of 5′-ATA GTT TAC GTG GCA T-3′ (SEQ ID NO: 51) in the DREB1A promoter was chosen as the target DNA by the artificial ZFP, and it was divided into two 10-bp DNAS, 5′-ATA GTT TAC G-3′ (Target A)(SEQ ID NO: 52) and 5′-TAC GTG GCA T-3′ (Target B)(SEQ ID NO: 53). As described in Example 7, DNA of a 2-finger ZFP for Target B (Zif A) and DNA of a 3-finger ZFP for Target A (Zif B) were prepared. Since the 3′ end of the ZifA DNA is ligated with 5′ end of the ZifB DNA, the Zif A DNA was amplified by PCR with primers OTS-007 and OTS-430 and the ZifB DNA with primers OTS-431 and OTS-008. The reactions were analyzed on a 2% agarose gel and produced the expected DNAs for 2- and 3-fingered ZFPs for ZifA and ZifB, respectively.

2) BsaI digestion

Both PCR products (0.5 μg of each) were digested at 50° C. for 1 hr in the 60 μl reaction buffer containing 20 units of BsaI endonuclease enzyme. After purifying with a ChromaSpin+TE-100 column, phenol extraction was performed to remove BsaI. The two digested DNA fragments were directly ligated using a DNA ligase enzyme (16° C., overnight). The reaction was analyzed on a 2% agarose gel and more than 80% of the product was the expected ligation product. The mixture was used for cloning into a pET-21a vector, and sequencing confirmed that the 5-finger domains were assembled in correct orientation.

| | |
|---|---|
| OTS-430: 5′-TTCAGGGCGGTCTCTCGGCTTCTCGCCAGTGTGAGTACGCTGATG-3′ | (SEQ ID NO:54) |
| (underlined nucleotides are the BsaI site). | |
| OTS-431: 5′-CGAATTCGGGTCTCAGCCGTATAAATGTCCGGAATGTGGTAAAA-3′ | (SEQ ID NO:55) |
| (underlined nucleotides are the BsaI site). | |

### EXAMPLE 9

### Modular Assembly of Six-Finger ZFPs

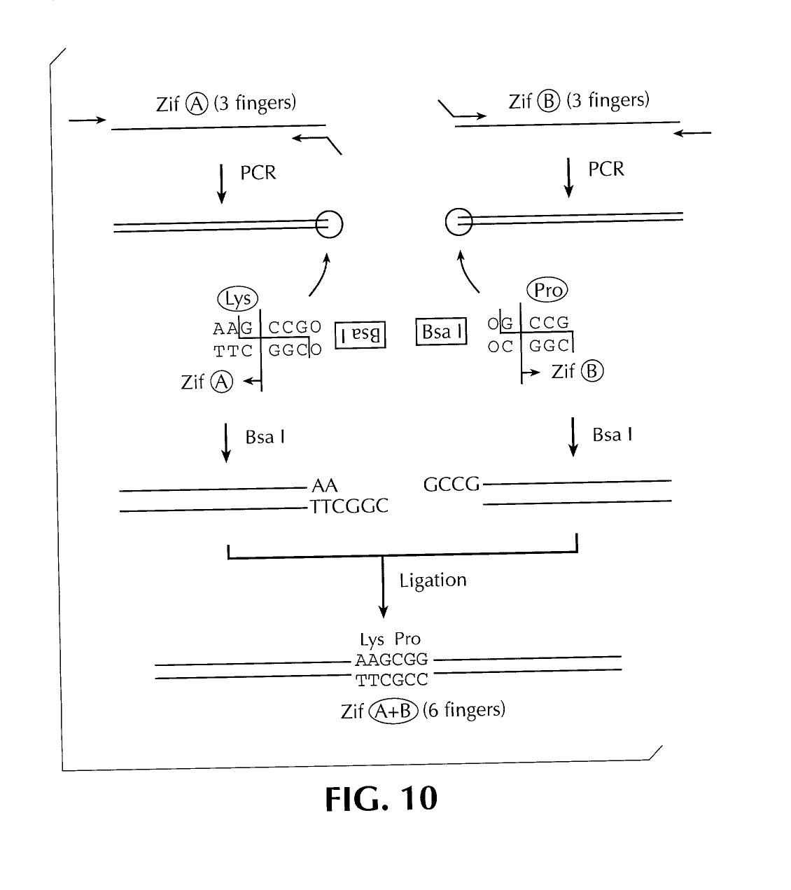

FIG. 10 shows a method of assembling 6-finger ZFPs. For example, a 3-finger DNA is amplified from the DNA of a 3-finger protein Zif-A by PCR primers OTS-007 and OTS-429, and a second 3-finger DNA is amplified from DNA of the 3-finger protein Zif-B by OTS-431 and OTS-008.

OTS-429:

5′-TGCGGCCGGGTCTCTCGGCTTCTCCCCCGTGTGCGTGCGTTGGTG-3′ (SEQ ID NO: 56) (underlined nucleotides are the BsaI site).

After amplification, the DNA fragments are digested with BsaI, which produces 5′-CGGC-3′ and 5′-GCCG-3′ sticky ends from ZifA and ZifB, respectively (FIG. 10). These sticky ends are complementary to each other, and the two digested DNA fragments can be assembled in correct orientation by a DNA ligase enzyme e.g., T4 DNA ligase. By using different primer sets, 4- and 5-finger proteins are prepared.

### EXAMPLE 10

### Assembly of Six-Finger Domains Into ZFPs

A 6-finger ZFP was designed to target the whole L1 site of BCTV (Clone 5, Table 5).

1) Preparation of two 3-finger DNAs

The L1 target site is 5′-TTG GGT GCT TTG GGT GCT C-3′ (SEQ ID NO: 57), and was divided into two 10-bp DNAs, 5′-TTG GGT GCT T-3′ (Target A)(SEQ ID NO: 58) and 5′-TTG GGT GCT C-3′ (Target B)(SEQ ID NO: 59), for ZFP design. DNAs of a 3-finger protein targeting Target B (ZifA) and another 3-finger protein binding to Target A (ZifB) were prepared according to the method described in Example 7 using PCR with primers OTS-007 and OTS-429 for ZifA, and with primers OTS-431 and OTS-008 for ZifB. The reaction was analyzed on a 2% agarose gel and the expected DNA fragments were obtained.

2) BsaI Digestion

Both PCR products (0.5 μg of each) were digested at 50° C. for 1 hr in the 60 μl reaction buffer containing 20 units of BsaI endonuclease enzyme. After purifying with a ChromaSpin+TE-100 column, phenol extraction was performed to remove BsaI. The two digested DNA fragments were directly ligated using a DNA ligase enzyme (16° C., overnight). The reaction was analyzed on a 2% agarose gel and more than 80% of the product was the expected ligation product. The mixture was used for cloning into a pET-21a vector, and it was confirmed that the 6-finger domains were assembled in correct orientation.

### EXAMPLE 11

### High Affinity 6-finger ZFPs

As described in Example 10, the DNA of Clone 5 was cloned into the EcoRI/HindIII sites of an E. coli expression vector of pET-21a. After expression in an E. coli strain BL21(DE3) pLysS, the protein was purified >95% homogeneous as judged by SDS/PAGE.

To determine the affinity of the artificial ZFP Clone 5, a gel shift assay was performed using a radiolabeled L1 target DNA duplex,

5′-TATATATATTGGGTGCTTTGGGTGCTCTATATATA-3′. (SEQ ID NO: 60) The concentrations of Clone 5 were 0, 0.003, 0.01, 0.03, 0.1 and 1 nM. The ZFPs were preincubated on ice for 40 minutes in 10 μl of 10 mM Tris-HCl, pH 7.5/100 mM NaCl/1 mM MgCl₂/0.1 mM ZnCl₂/1 mg/ml BSA/10% glycerol containing the radiolabeled probe (0.03 fmol per 10 μl of buffer). 1 μg of poly(dA-dT)₂ was then added, and incubation was continued for 20 minutes before loading onto a 6% nondenaturing polyacrylamide gel (0.5×TB) and electrophoresing at 140 V for 2 hr at 4° C. The radioactive signals were exposed on x-ray films.

For Clone 5, the vast majority of the DNA probe is bound to the protein even at 3 pM. Hence, the dissociation constant is less than 3 pM. Two additional ZFPs were synthesized (Clones 6 and 7; Table 5) and produced proteins with similar high affinities.

### EXAMPLE 12

### Design, Production and Analysis of Additional ZFPs

Additional multi-fingered ZFPs were designed and synthesized according to the strategy of Examples 7 and 9 using the Sp1C domain 2 framework and the amino acids at positions −1, 2, 3 and 6 as shown in Table 2. The targets sequences for each ZFP and the dissociation constant of the ZFP for its target is provided in Table 5.

In tomato golden mosaic virus (TGMV) and beet curly top virus (BCTV) genomes, the target sites are critical sites for the gemini viral replication (Clones 1 and 2). Other target sites are the sequences found around 50 to 100-bp upstream from TATA box in promoters of plant genes, Arabidopsis thaliana DREBIA (drought tolerance gene; Clone 3) and NIM1 (systemic acquired resistance; Clone 4).

In these experiments, the coding regions of designed ZFPs were cloned into the EcoRI and HindIII sites of expression vector pET-21a (Novagen). Resulting plasmids were then introduced into E. coli BL21(DE3)pLysS for protein overexpression. A 60-ml culture was grown to OD₆₀₀=0.6-0.75 at 37° C., induced with 1 mM IPTG for 3 hr, and lysed using a ultrasonicator in cold lysis buffer [100 mM Tris-HCl, pH8.0/1 M NaCl/1 mM ZnCl₂/5 mM dithiothreitol containing one tablet of Complete, Mini, EDTA-free (Roche Molecular Biochemicals) per 10 ml lysis buffer. After treatment with polyethyleneimine (pH 7.0, 0.6%) and precipitated with 40% (NH₄)₂SO₄, the resulting pellet was redissolved in 50 mM Tris-HCl, pH 8.0/100 mM NaCl/0.1 mM ZnCl₂/5 mM dithiothreitol buffer and purified by chromatography on a Bio-Rex 70 column, eluting 300 mM NaCl buffer. All purified proteins were >95% homogeneous as judged by SDS/PAGE. Protein concentration was determined using Protein Assay ESL (Roche Molecular Biochemicals).

For the DNA binding assays, twenty six base-pair synthetic oligonucleotides, labeled at the 5′-end with [γ-³²P]ATP, were used in the gel-retardation assays. Probes for ZFPs with more than 5 finger domains were labeled with Klenow Fragment and [α-³²P]dATP and [α-³²P]dTTP to obtain high radioactivity. The ZFPs were preincubated on ice for 40 minutes in 10 μl of 10 mM Tris-HCl, pH 7.5/100 mM NaCl/1 mM MgCl₂/0.1 mM ZnCl₂/1 mg/ml BSA/10% glycerol containing the radiolabeled probe (1 fmol per 10 μl of buffer) and 1 μg of poly(dA-dT)₂ was then added, and incubation was continued for 20 minutes before loading onto a 6% nondenaturing polyacrylamide gel (0.5×TB) and electrophoresing at 140 V for 2 hr at 4° C. For multi-finger proteins, 0.03 fmol of radiolabeled probes were used. The radioactive signals were quantitated with a PhosphorImager (Molecular Dynamics) and exposed on x-ray films. The dissociation constants were calculated by curve fitting with the KALEIDAGRAPH program (Synergy Software).

Gel shift assays were performed with the designed 3-finger and 6-finger proteins and the K_{d} values were calculated. The measured K_{d} for clones 1-4 was 18, 15, 11 and 23 nM respectively. For Clones 5-7, the K_{d}s were all less than 3 pM.

TABLE 5

| | | Amino Acids Used for Recognition | | |
|---|---|---|---|---|
| No. | Target Sequence | Zif1 | Zif2 | Zif3 |
| | | −1 2 3 6 | −1 2 3 6 | −1 2 3 6 |
| 1 | 5′ AGT AAG GTA G 3′ | GlnAspSerArg | ArgAspAsnGln | ThrThrHisGln |
| 2 | 5′ TTG GGT GCT C 3′ | ThrSerAspArg | ThrAspHisArg | ArgAspSerThr |
| 3 | 5′ TAG GTG GCA T 3′ | GlnAsnAspArg | ArgAspSerArg | GluAspAsnThr |
| 4 | 5′ GGA GAT GAT A 3′ | ThrThrAsnArg | ThrAspAsnArg | GlnAspHisArg |
| 5 | 5′ TTG GGT GCT TTG GGT GCT C 3′ | | | |
| 6 | 5′ AGT AAG GTA GGA GAT GAT A 3′ | | | |
| 7 | 5′ TAC GTG GCA TTG GGT GCT C 3′ | | | |

The target sequences of Clones 1-7 are designated as SEQ ID NOS: 61-67, respectively.

What is claimed is:

1. A method of making a nucleic acid encoding a zinc finger protein (ZFP) comprising three contiguous zinc fingers domains, each separated from the other by no more than 10 amino acids,
(a) preparing a mixture, under conditions for performing a polymerase-chain reaction (PCR), comprising:
(i) a first double-stranded oligonucleotide encoding a first zinc finger domain,
(ii) a second double-stranded oligonucleotide encoding a second zinc finger domain,
(iii) a third double-stranded oligonucleotide encoding a third zinc finger,
(iv) a first PCR primer complementary to the 5′ end of the first oligonucleotide,
(v) a second PCR primer complementary to the 3′ end of the third oligonucleotide,
wherein the 3′ end of the first oligonucleotide is sufficiently complementary to the 5′ end of the second oligonucleotide to prime synthesis of said second oligonucleotide therefrom,
wherein the 3′ end of the second oligonucleotide is sufficiently complementary to the 5′ end of the third oligonucleotide to prime synthesis of said third oligonucleotide therefrom, and
wherein the 3′ end of the first oligonucleotide is not complementary to the 5′ end of the third oligonucleotide and the 3′ end of the second oligonucleotide is not complementary to the 5′ end of the first oligonucleotide;
(b) subjecting the mixture to a PCR; and
(c) recovering the nucleic acid encoding the three zinc finger domains and preparing a nucleic acid encoding said ZFP.

2. A method of making a nucleic acid encoding a zinc finger protein (ZFP) comprising three zinc fingers domains, each domain independently represented by the formula
-X₃-Cys-X₂₋₄-Cys-X₁₂-His-X₃₋₅-His-X₄-,
and said domains, independently, covalently joined with from 0 to 10 amino acid residues which comprises:
(a) preparing a mixture, under conditions for performing a polymerase-chain reaction (PCR), comprising:
(i) a first double-stranded oligonucleotide encoding a first zinc finger domain,
(ii) a second double-stranded oligonucleotide encoding a second zinc finger domain,
(iii) a third double-stranded oligonucleotide encoding a third zinc finger,
(iv) a first PCR primer complementary to the 5′ end of the first oligonucleotide,
(v) a second PCR primer complementary to the 3′ end of the third oligonucleotide,
wherein the 3′ end of the first oligonucleotide is sufficiently complementary to the 5′ end of the second oligonucleotide to prime synthesis of said second oligonucleotide therefrom,
wherein the 3′ end of the second oligonucleotide is sufficiently complementary to the 5′ end of the third oligonucleotide to prime synthesis of said third oligonucleotide therefrom, and
wherein the 3′ end of the first oligonucleotide is not complementary to the 5′ end of the third oligonucleotide and the 3′ end of the second oligonucleotide is not complementary to the 5′ end of the first oligonucleotide;
(b) subjecting the mixture to a PCR; and
(c) recovering the nucleic acid encoding the three zinc finger domains and preparing a nucleic acid encoding said ZFP.

3. The method of claim 2, wherein the first and second PCR primers independently include a restriction endonuclease recognition site.

4. The method of claim 3, wherein said restriction endonuclease recognition site is for BbsI, BsaI, BsmBI, or BspMI.

5. The method of claim 4, wherein said restriction endonuclease recognition site is for BsaI.

6. A method of making a nucleic acid encoding a zinc finger protein (ZFP) comprising four or more contiguous zinc fingers domains, each separated from the other by no more than 10 amino acids,
(a) preparing a first nucleic acid according to the method of claim 3, wherein said second PCR primer includes a first restriction endonuclease recognition site;
(b) preparing a second nucleic acid according to the method of claim 3,
wherein said first and second PCR primers are complementary to the 5′ and 3′ ends, respectively, of the number of zinc finger domains selected for amplification,
wherein said first PCR primer includes a restriction endonuclease recognition site that, when subjected to cleavage by its corresponding restriction endonuclease, produces an end having a sequence which is complementary to and can anneal to, the end produced when said second PCR primer of step (a) is subjected to cleavage by its corresponding restriction endonuclease and
wherein said second PCR primer of step (b), optionally, includes a second restriction enzyme recognition site that, when subjected to cleavage produces an end that differs from and is not complementary to that produced from the first restriction endonuclease recognition site;
(c) optionally, preparing one or more additional nucleic acids by the method of claim 3,
wherein said first and second PCR primers are complementary to the 5′ and 3′ ends, respectively, of the number of zinc finger domains selected for amplification,
wherein said first PCR primer for each additional nucleic acid includes a restriction endonuclease recognition site that, when subjected to cleavage by its corresponding restriction endonuclease, produces an end having a sequence which is complementary to and can anneal to the end produced when the second PCR primer used for preparation of the second nucleic acid, or for the additional nucleic acid that is immediately upstream of the additional nucleic acid, is subjected to cleavage by its corresponding restriction endonuclease, and
wherein said second PCR primer for each additional nucleic acid, optionally, includes a restriction endonuclease recognition site that, when subjected to cleavage produces an end that differs from and is not complementary to any previously used;
(d) cleaving said first nucleic acid, said second nucleic acid and said additional nucleic acids, if prepared, with their corresponding restriction endonucleases to produce cleaved first, second and additional, if prepared, nucleic acids; and
(e) ligating said cleaved first, second and additional, if prepared, nucleic acids to produce the nucleic acid encoding a zinc finger protein (ZFP) having four or more zinc fingers domains.

7. A method of making a nucleic acid encoding a zinc finger protein (ZFP) having four or more zinc fingers domains, each domain independently represented by the formula
-X₃-Cys-X₂₋₄-Cys-X₁₂-His-X₃₋₅-His-X₄-,
and said domains, independently, covalently joined with from 0 to 10 amino acid residues which comprises:
(a) preparing a first nucleic acid according to the method of claim 3, wherein said second PCR primer includes a first restriction endonuclease recognition site;
(b) preparing a second nucleic acid according to the method of claim 3,
wherein said first and second PCR primers are complementary to the 5′ and 3′ ends, respectively, of the number of zinc finger domains selected for amplification,
wherein said first PCR primer includes a restriction endonuclease recognition site that, when subjected to cleavage by its corresponding restriction endonuclease, produces an end having a sequence which is complementary to and can anneal to, the end produced when said second PCR primer of step (a) is subjected to cleavage by its corresponding restriction endonuclease and
wherein said second PCR primer of step (b), optionally, includes a second restriction enzyme recognition site that, when subjected to cleavage produces an end that differs from and is not complementary to that produced from the first restriction endonuclease recognition site;
(c) optionally, preparing one or more additional nucleic acids by the method of claim 3,
wherein said first and second PCR primers are complementary to the 5′ and 3′ ends, respectively, of the number of zinc finger domains selected for amplification,
wherein said first PCR primer for each additional nucleic acid includes a restriction endonuclease recognition site that, when subjected to cleavage by its corresponding restriction endonuclease, produces an end having a sequence which is complementary to and can anneal to the end produced when the second PCR primer used for preparation of the second nucleic acid, or for the additional nucleic acid that is immediately upstream of the additional nucleic acid, is subjected to cleavage by its corresponding restriction endonuclease, and
wherein said second PCR primer for each additional nucleic acid, optionally, includes a restriction endonuclease recognition site that, when subjected to cleavage produces an end that differs from and is not complementary to any previously used;
(d) cleaving said first nucleic acid, said second nucleic acid and said additional nucleic acids, if prepared, with their corresponding restriction endonucleases to produce cleaved first, second and additional, if prepared, nucleic acids; and
(e) ligating said cleaved first, second and additional, if prepared, nucleic acids to produce the nucleic acid encoding a zinc finger protein (ZFP) having four or more zinc fingers domains.

8. The method of claim 6 or 7, wherein each restriction endonuclease is, independently, BbsI, BsaI, BsmBI, or BspMI, and each endonuclease produces a unique pair of cleavable, anneable ends.

9. The method of claim 6 or 7, wherein the restriction endonuclease is BsaI and each use thereof produces a unique pair of cleavable, anneable ends.

10. The method of claim 6 or 7, wherein step (c) is omitted and said nucleic acid encoding a zinc finger protein (ZFP) has four, five or six zinc finger domains.

11. The method of claim 10, wherein said restriction endonuclease is BbsI, BsaI, BsmBI, or BspMI.

12. The method of claim 10, wherein said restriction endonuclease is BsaI.

13. The method of claim 6 or 7, wherein the PCR primers for the second nucleic acid were selected to amplify three zinc finger domains, one additional nucleic acid is prepared by step (c), and said nucleic acid encoding a zinc finger protein (ZFP) has seven, eight or nine zinc finger domains.

14. The method of claim 13, wherein each restriction endonuclease is, independently, BbsI, BsaI, BsmBI, or BspMI, and each endonuclease produces a unique pair of cleavable, anneable ends.

15. The method of claim 13, wherein the restriction endonuclease is BsaI and each use thereof produces a unique pair of cleavable, anneable ends.

16. The method of any one of claims 1, 2, 6 or 7, wherein the sequences of said oligonucleotides are selected to provide for optimal codon usage for an organism.

17. The method of claim 16, wherein said organism is a bacterium, a fungus, a yeast, an animal, an insect or a plant.

18. The method of claim 17, wherein said bacterium is E. coli.

19. The method of claim 17, wherein said animal is a human or a commercial animal.

20. The method of claim 17, wherein said plant is a cereal plant.

21. The method of claim 17, wherein said plant is rice, tomato or corn.

22. The method of claim 17, wherein said plant is a transgenic plant.

23. An expression vector comprising a nucleic acid prepared by the method of any one of claims 1, 2, 6 or 7.

24. A host cell comprising the expression vector of claim 23.

25. A method of preparing a zinc finger protein which comprises
(a) culturing the host cell of claim 24 for a time and under conditions to express said ZFP; and
(b) recovering said ZFP.

26. The method of claim 10, wherein the sequences of said oligonucleotides are selected to provide for optimal codon usage for an organism.

27. The method of claim 26, wherein said organism is a bacterium, a fungus, a yeast, an animal, an insect or a plant.

28. An expression vector comprising a nucleic acid prepared by the method of claim 10.

29. A host cell comprising the expression vector of claim 28.

30. A method of preparing a zinc finger protein which comprises
(a) culturing the host cell of claim 29 for a time and under conditions to express said ZFP; and
(b) recovering said ZFP.

31. The method of claim 13, wherein the sequences of said oligonucleotides are selected to provide for optimal codon usage for an organism.

32. The method of claim 31, wherein said organism is a bacterium, a fungus, a yeast, an animal, an insect or a plant.

33. An expression vector comprising a nucleic acid prepared by the method of claim 13.

34. A host cell comprising the expression vector of claim 33.

35. A method of preparing a zinc finger protein which comprises
(a) culturing the host cell of claim 34 for a time and under conditions to express said ZFP; and
(b) recovering said ZFP.
